(19) United States
(12) Patent Application Publication
(10) Pub. No.: US 2025/0012804 A1
(43) Pub. Date: Jan. 9, 2025

(54) USE OF MICROVESICLES FOR BENIGN COLORECTAL POLYPS AND COLORECTAL CANCER SCREENING

(71) Applicant: UCL BUSINESS LTD, London (GB)

(72) Inventors: Rijan GURUNG, London (GB); Mohammad EDDAMA, London (GB); Lucie CLAPP, London (GB); Richard COHEN, London (GB); Marilena LOIZIDOU, London (GB)

(21) Appl. No.: 18/552,977

(22) PCT Filed: Mar. 31, 2022
(86) PCT No.: PCT/GB2022/050811

§ 371 Date: Sep. 28, 2023

(30) Foreign Application Priority Data
Mar. 31, 2021 (GB) 2104600.8

(51) Int. Cl. G01N 33/574

(52) U.S. Cl. CPC G01N 33/57419; G01N 33/57473; G01N 33/57488

(57) ABSTRACT

The present invention is concerned with methods for assessing whether an individual has colorectal cancer (CRC) or is at risk of developing CRC. The methods involve providing a sample of microvesicles (MVs) which has been obtained from the plasma of the individual; determining the concentration of MVs in the individual's plasma and other bodily fluids; and classifying the individual as having benign colorectal polyps (BCRPs) or CRC when the concentration of MVs in the individual's plasma is statistically significantly higher compared to control. The methods may further involve assessing whether an individual has CRC by determining the concentration of MVs which test positive for the detectable expression, preferably the detectible surface expression, of one or more biomarkers. The methods may further involve assessing whether an individual has BCRPs by determining the concentration of MVs which test positive for the detectable surface expression of one or more biomarkers. The methods may further involve assessing whether an individual has CRC by determining the concentration of one or more blood proteins, one or more blood cell types, or one or more compounds found in blood.

### FIELD OF THE INVENTION

The present invention is concerned with methods for assessing whether an individual has colorectal cancer (CRC) or is at risk of developing CRC. The methods involve providing a sample of microvesicles (MVs) which has been obtained from the plasma of the individual; determining the concentration of MVs in the individual's plasma; and classifying the individual as having benign colorectal polyps (BCRPs) or CRC when the concentration of MVs in the individual's plasma is statistically significantly higher compared to control.

The methods may further involve assessing whether an individual has CRC by determining the concentration of MVs which test positive for the detectable expression, preferably detectable surface expression, of one or more biomarkers.

The methods may further involve assessing whether an individual has BCRPs by determining the concentration of MVs which test positive for the detectable expression, preferably detectable surface expression, of one or more biomarkers.

The methods may further involve assessing whether an individual has CRC by determining the concentration of one or more blood proteins, one or more blood cell types, or one or more compounds found in blood.

### BACKGROUND TO THE INVENTION

Colorectal cancer (CRC) is one of the leading cause of cancer deaths worldwide. No single risk factor accounts for the majority of colorectal cancer cases, though many have been associated with the disease including family history of colorectal cancer, inflammatory bowel disease, smoking, excessive alcohol consumption, high consumption of processed and red meat, diabetes, and obesity (Kolligs, F. T. (2016). Diagnostics and epidemiology of colorectal cancer. Visc. Med. 32: 158-164). Lower survival in the United Kingdom compared to European countries has been attributed to late stage-presentation and diagnosis.

Surviving CRC is significantly improved by early diagnosis. For example, 5-year survival rate of patients who are diagnosed at stage 1 is approximately 96%. This rate decreases if patients are diagnosed at a more advanced stage: for stages 2, 3 and 4, 5-year survival rates are 87%, 66%, and 14% respectively. Thus, screening programs aim to detect CRC early in order to improve mortality. The evidence from faecal occult blood (FOB) screening programme shows that early diagnosis of CRC before symptoms occur have significantly reduced overall mortality by 16-22%.

Similarly, screening with flexible sigmoidoscopy reduces CRC mortality specific to the distal colon by 22-31%. The significant reduction in mortality in the screened population is not only due to early diagnosis, but also due to polypectomy, for those who have positive FOB and subsequently undergo colonoscopy or those who undergo flexible sigmoidoscopy.

The evidence suggests that benign colorectal polyps (BCRP) if untreated often progresses to malignant tumours. It has been demonstrated that a positive FOB test infers a 40% chance of the patient having a benign polyp. One randomised controlled trial (RCT) found a reduction in the incidence of CRC in a screened population, presumably due to polypectomy.

The primary method of investigation for colorectal cancer remains colonoscopy and histological confirmation of diagnosis via biopsy (Brenner, H., Kloor, M., and Pox, C. P. (2014). Colorectal cancer. Lancet 383: 1490-1502). Computerised tomography (CT)-colonography is a more recent radiological investigation and may be used as an alternative to colonoscopy. These procedures are invasive, require a multidisciplinary team, and resources needed to perform them are usually scarce in most clinical settings. Several biomarkers are currently available for evaluating symptomatic patients which include blood and faecal tests, serum carcinoembryonic antigen and faecal calprotectin, but they have suboptimal and often poor sensitivity and specificity (Vega, P., Valentin, F., and Cubiella, J. (2015). Colorectal cancer diagnosis: Pitfalls and opportunities. World J. Gastrointest. Oncol. 7: 422-33).

Accordingly improved screening methods are required that are suitable for assessing at an early stage whether individuals have CRC or are at risk of developing CRC. More cost-effective, efficient, minimally invasive, sensitive and specific screening methods are required.

### SUMMARY OF THE INVENTION

The present inventors have discovered that microvesicles (MVs) are statistically significantly elevated in the plasma of colorectal cancer (CRC) and benign colorectal polyp (BRCP) patients compared to healthy control subjects and patients with other bowel conditions such as diverticular disease and inflammatory bowel disease. Furthermore, the present inventors have identified several proteins which show statistically significantly elevated levels of expression in MVs compared to healthy control subjects and patients with other bowel conditions. Each one of these proteins, alternatively referred to as biomarkers, can be used in isolation or together in any combination and provide statistically robust assays, in terms of specificity and sensitivity, classifying an individual as having BCRPs or CRC.

The data produced by the inventors indicate that MVs are highly predictive of colorectal neoplasms. In fact, they have a much higher positive and negative predictive value than those of the currently used faecal screening test (gold standard) for bowel cancer.

The present disclosure demonstrates the surprising role of MVs as a screening tool for BCRP and CRC. Accordingly, the present disclosure provides screening methods that are suitable for assessing at an early stage whether individuals have CRC or are at risk of developing CRC, wherein the methods are cost-effective, efficient, minimally invasive, sensitive and specific.

Accordingly, the invention provides a method of assessing whether an individual has colorectal cancer (CRC) or is at risk of developing CRC, the method comprising:
1) providing a sample of microvesicles (MVs) which has been obtained from the plasma of the individual;
2) determining the concentration of MVs in the individual's plasma; and
3) classifying the individual as having benign colorectal polyps (BCRPs) or CRC when the concentration of MVs in the individual's plasma is statistically significantly higher compared to control.

In the method of the invention described above, the plasma concentration of MVs in the control may be 124 MVs/μL or less.

In any one of the methods of the invention described above, when the plasma concentration of MVs is 144 MVs/μL or more the individual may be identified as having BCRPs or CRC, wherein the method has a receiver operating characteristics (ROC) sensitivity of 100% and a specificity of 59%.

In any one of the methods of the invention described above, when the plasma concentration of MVs is 244 MVs/μL or more the individual may be identified as having BCRPs or CRC, wherein the method has a ROC sensitivity of 100% and a specificity of 93%.

The method of the invention may be performed as described above and may further comprise:
1) determining the concentration of MVs in the individual's plasma which test positive for the detectable expression, preferably detectable surface expression, of one or more biomarkers; and
2) classifying the individual as having BCRPs or CRC when the concentration of biomarker positive MVs in the individual's plasma is statistically significantly higher compared to control and/or when the concentration of biomarker positive MVs in the individual's plasma exceeds a threshold value.

In any one of the methods of the invention described above, wherein:
1) the one or more biomarkers comprises CEA, the individual may be classified as having CRC when the concentration of MVs which test positive for the detectable expression of CEA exceeds a threshold value, and wherein the method has a ROC sensitivity of 100% and a specificity of 60% or more, preferably wherein the threshold value is 10 MVs/μL or more; and/or
2) the one or more biomarkers comprises A33, the individual may be classified as having CRC when the concentration of MVs which test positive for the detectable expression of A33 exceeds a threshold value, and wherein the method has a ROC sensitivity of 100% and a specificity of 13% or more, preferably wherein the threshold value is 13 MVs/μL or more; and/or
3) the one or more biomarkers comprises LGR5, the individual may be classified as having CRC when the concentration of MVs which test positive for the detectable expression of LGR5 exceeds a threshold value, and wherein the method has a ROC sensitivity of 96% or more and a specificity of 53% or more, preferably wherein the threshold value is 19 MVs/μL or more; and/or
4) the one or more biomarkers comprises EPhB2, the individual may be classified as having CRC when the concentration of MVs which test positive for the detectable expression of EPhB2 exceeds a threshold value, and wherein the method has a ROC sensitivity of 100% and a specificity of 13% or more, preferably wherein the threshold value is 52 MVs/μL or more; and/or
5) the one or more biomarkers comprises ICAM-1, the individual may be classified as having CRC when the concentration of MVs which test positive for the detectable expression of ICAM-1 exceeds a threshold value, and wherein the method has a ROC sensitivity of 96% or more and a specificity of 47% or more, preferably wherein the threshold value is 12 MVs/μL or more; and/or
6) the one or more biomarkers comprises CD31, the individual may be classified as having CRC when the concentration of MVs which test positive for the detectable expression of CD31 exceeds a threshold value, and wherein the method has a ROC sensitivity of 100% and a specificity of 60% or more, preferably wherein the threshold value is 38 MVs/μL or more; and/or
7) the one or more biomarkers comprises CD42a, the individual may be classified as having CRC when the concentration of MVs which test positive for the detectable expression of CD42a exceeds a threshold value, and wherein the method has a ROC sensitivity of 100% and a specificity of 20% or more, preferably wherein the threshold value is 9 MVs/μL or more; and/or
8) the one or more biomarkers comprises CD31+/CD42a−, the individual may be classified as having CRC when the concentration of MVs which test positive for the detectable expression of CD31+/CD42a− exceeds a threshold value, and wherein the method has a ROC sensitivity of 96% or more and a specificity of 87% or more, preferably wherein the threshold value is 38 MVs/μL or more; and/or
9) the one or more biomarkers comprises CK20, the individual may be classified as having CRC when the concentration of MVs which test positive for the detectable expression of CK20 exceeds a threshold value, and wherein the method has a ROC sensitivity of 100% and a specificity of 47% or more, preferably wherein the threshold value is 8 MVs/μL or more; and/or
10) the one or more biomarkers comprises CK7, n the individual may be classified as having CRC when the concentration of MVs which test positive for the detectable expression of cytokeratin 7 exceeds a threshold value, and wherein the method has a ROC sensitivity of 100% and a specificity 47% or more, preferably wherein the threshold value is 16 MVs/μL or more; and/or
11) the one or more biomarkers comprises CK20+/CK7−, the individual may be classified as having CRC when the concentration of MVs which test positive for the detectable expression of Ck20+/CD7− exceeds a threshold value, and wherein the method has a ROC sensitivity of 100% and a specificity of 33% or more, preferably wherein the threshold value is 4 MVs/μL or more; and/or
12) the one or more biomarkers comprises HLA-DR, the individual may be classified as having CRC when the concentration of MVs which test positive for the detectable expression of HLA-DR exceeds a threshold value, and wherein the method has a ROC sensitivity of 100% and a specificity of 27% or more, preferably wherein the threshold value is 26 MVs/μL or more; and/or
13) the one or more biomarkers comprises CD147, the individual may be classified as having CRC when the concentration of MVs which test positive for the detectable expression of CD147 exceeds a threshold value, and wherein the method has a ROC sensitivity of 96% or more and a specificity of 27% or more, preferably wherein the threshold value is 17 MVs/μL or more.

In any one of the methods of the invention described above, wherein:
1) the one or more biomarkers comprises CEA, the individual may be classified as having BCRPs when the concentration of MVs which test positive for the detectable expression of CEA exceeds a threshold value, and wherein the method has a ROC sensitivity of 100% and a specificity of 40% or more, preferably wherein the threshold value is 6.37 MVs/μL or more; and/or
2) the one or more biomarkers comprises LGR5, the individual may be classified as having BCRPs when the concentration of MVs which test positive for the detectable expression of LGR5 exceeds a threshold value, and wherein the method has a ROC sensitivity of 100% and a specificity of 60% or more, preferably wherein the threshold value is 28.4 MVs/μL or more; and/or
3) the one or more biomarkers comprises EPhB2, the individual may be classified as having BCRPs when the concentration of MVs which test positive for the detectable expression of EPhB2 exceeds a threshold value, and wherein the method has a ROC sensitivity of 100% and a specificity of 47% or more, preferably wherein the threshold value is 1.07 MVs/μL or more; and/or
4) the one or more biomarkers comprises ICAM-1, the individual may be classified as having BCRPs when the concentration of MVs which test positive for the detectable expression of ICAM-1 exceeds a threshold value, and wherein the method has a ROC sensitivity of 100% and a specificity of 13% or more, preferably wherein the threshold value is 3.6 MVs/μL or more; and/or
5) the one or more biomarkers comprises CD31, the individual may be classified as having BCRPs when the concentration of MVs which test positive for the detectable expression of CD31 exceeds a threshold value, and wherein the method has a ROC sensitivity of 100% and a specificity of 87% or more, preferably wherein the threshold value is 72 MVs/μL or more; and/or
6) the one or more biomarkers comprises CD42a, the individual may be classified as having BCRPs when the concentration of MVs which test positive for the detectable expression of CD42a exceeds a threshold value, and wherein the method has a ROC sensitivity of 100% and a specificity of 47% or more, preferably wherein the threshold value is 25 MVs/μL or more; and/or
7) the one or more biomarkers comprises CD31+/CD42a−, the individual may be classified as having BCRPs when the concentration of MVs which test positive for the detectable expression of CD31+/CD42a− exceeds a threshold value, and wherein the method has a ROC sensitivity of 100% and a specificity of 87% or more, preferably wherein the threshold value is 44 MVs/μL or more; and/or
8) the one or more biomarkers comprises CK7, the individual may be classified as having BCRPs when the concentration of MVs which test positive for the detectable expression of CK7 exceeds a threshold value, and wherein the method has a ROC sensitivity of 100% and a specificity of 33% or more, preferably wherein the threshold value is 11 MVs/μL or more; and/or
9) the one or more biomarkers comprises HLA-DR, the individual may be classified as having BCRPs when the concentration of MVs which test positive for the detectable expression of HLA-DR exceeds a threshold value, and wherein the method has a ROC sensitivity of 100% and a specificity of 7% or more, preferably wherein the threshold value is 17 MVs/μL or more; and/or
10) the one or more biomarkers comprises CD147, the individual may be classified as having BCRPs when the concentration of MVs which test positive for the detectable expression of CD147 exceeds a threshold value, and wherein the method has a ROC sensitivity of 100% and a specificity of 13% or more, preferably wherein the threshold value is 7.4 MVs/μL or more.

In any one of the methods of the invention described above, the individual may be classified as having BCRPs when the concentration of MVs which test positive for the detectable expression of all biomarkers comprising the Component Factor 2 (CF2) group of biomarkers exceeds a threshold value and wherein the method has a ROC sensitivity of 100% and a specificity of 63% or more, preferably wherein the threshold value is 222 MVs/μL or more.

In any one of the methods of the invention described above, the individual may be classified as having BCRPs when the concentration of MVs which test positive for the detectable expression of all biomarkers comprising the Component Factor 1 (CF1) group of biomarkers exceeds a threshold value and wherein the method has a ROC sensitivity of 100% and a specificity of 100%, preferably wherein the threshold value is 761 MVs/μL or more.

In any one of the methods of the invention described above, the individual may be classified as having CRC when the concentration of MVs which test positive for the detectable expression of all biomarkers comprising the Component Factor 2 (CF2) group of biomarkers exceeds a threshold value and wherein the method has a ROC sensitivity of 100% and a specificity of 50% or more, preferably wherein the threshold value is 157 MVs/μL or more.

In any one of the methods of the invention described above, the individual may be classified as having CRC when the concentration of MVs which test positive for the detectable expression of all biomarkers comprising the Component Factor 1 (CF1) group of biomarkers exceeds a threshold value and wherein the method has a ROC sensitivity of 100% and a specificity of 56% or more, preferably wherein the threshold value is 439 MVs/μL or more.

In any one of the methods of the invention described above, the method may further comprise providing a sample of blood which has been obtained from the individual, determining the concentration of a protein in the individual's blood and classifying the individual as having CRC when the concentration of the protein is below a threshold value, wherein the protein is haemoglobin and wherein the method has a ROC sensitivity of 68% or more and a specificity of 100%. The individual may be distinguished as having CRC rather than BCRPs when the concentration of haemoglobin is 133 g/L or less, and wherein the method has a ROC sensitivity of 58.3% or more and a specificity of 90% or more; or the individual may be classified as having CRC when the concentration of haemoglobin is 124.5 g/L or less, and wherein the method has a ROC sensitivity of 68% or more and a specificity of 100%.

In any one of the methods of the invention described above, the method may further comprise providing a sample of blood which has been obtained from the individual, determining the concentration of a blood cell type in the individual's blood and classifying the individual as having CRC when the concentration of the blood cell type is above a threshold value, wherein the blood cell type is neutrophils and wherein the method has a ROC sensitivity of 47% or more and a specificity of 100%. The individual may be classified as having CRC when the concentration of neutrophils is 7.7×10⁹/L, or more, and wherein the method has a ROC sensitivity of 47% or more and a specificity of 100%.

In any one of the methods of the invention described above, the method may further comprise providing a sample of blood which has been obtained from the individual, determining the concentration of a blood cell type in the individual's blood and classifying the individual as having CRC when the concentration of the blood cell type is below a threshold value, wherein the blood cell type is lymphocytes and wherein the method has a ROC sensitivity of 47% or more and a specificity of 100%. The individual may be classified as having CRC when the concentration of lymphocytes is 1.16×10⁹/L or less, and wherein the method has a ROC sensitivity of 47% or more and a specificity of 100%.

In any one of the methods of the invention described above, the method may further comprise providing a sample of blood which has been obtained from the individual, determining the concentration of a protein in the individual's blood and classifying the individual as having CRC when the concentration of the protein is below a threshold value, wherein the protein is albumen and wherein the method has a ROC sensitivity of 63% or more and a specificity of 100%. The individual may be distinguished as having CRC rather than BCRPs when the concentration of albumen is 43.5 g/L or less, and wherein the method has a ROC sensitivity of 76.9% or more and a specificity of 90% or more; or wherein the individual is classified as having CRC when the concentration of albumen is 39.5 g/L or less, and wherein the method has a ROC sensitivity of 63% or more and a specificity of 100%.

In any one of the methods of the invention described above, the method may further comprise providing a sample of blood which has been obtained from the individual, determining the concentration of a protein in the individual's blood and classifying the individual as having CRC when the concentration of the protein is above a threshold value, wherein the protein is C-reactive protein (CRP) and wherein the method has a ROC sensitivity of 63% or more and a specificity of 100%. The individual may be distinguished as having CRC rather than BCRPs when the concentration of CRP is 19.65 mg/L or more, and wherein the method has a ROC sensitivity of 85.7% or more and a specificity of 90% or more; or wherein the individual is classified as having CRC when the concentration of CRP is 72.75 mg/L or more, and wherein the method has a ROC sensitivity of 63% or more and a specificity of 100%.

In any one of the methods of the invention described above, the method may further comprise providing a sample of blood which has been obtained from the individual, determining the concentration of a compound in the individual's blood and classifying the individual as having CRC when the concentration of the compound is below a threshold value, wherein the compound is urea and wherein the method has a ROC sensitivity of 58% or more and a specificity of 100%. The individual may be distinguished as having CRC rather than BCRPs when the concentration of urea is 5.5 mmol/L or less, and wherein the method has a ROC sensitivity of 54.55% or more and a specificity of 90% or more; or wherein the individual is classified as having CRC when the concentration of urea is 3.85 mmol/L or less, and wherein the method has a ROC sensitivity of 58% or more and a specificity of 100%.

In any one of the methods of the invention described above, the method may further comprise providing a sample of blood which has been obtained from the individual, determining the concentration of a compound in the individual's blood and classifying the individual as having CRC when the concentration of the compound is below a threshold value, wherein the compound is creatinine and wherein the method has a ROC sensitivity of 53% or more and a specificity of 100%. The individual may be distinguished as having CRC rather than BCRPs when the concentration of creatinine is 82.5 mmol/L or less, and wherein the method has a ROC sensitivity of 75% or more and a specificity of 90.0% or more; or wherein the individual is classified as having CRC when the concentration of creatinine is 63.5 μmol/L or less, and wherein the method has a ROC sensitivity of 53% or more and a specificity of 100%.

In any one of the methods of the invention described above, the method may further comprise providing a sample of blood which has been obtained from the individual, determining the concentration of MVs in the plasma of the individual which test positive for the detectable expression of a protein and classifying the individual as having CRC when the concentration of MVs positive for the protein in the plasma is above a threshold value, wherein the protein is carcinoembryonic antigen (CEA) and wherein the method has a ROC sensitivity of 25% or more and a specificity of 100%. The individual may be classified as having CRC when the concentration of CEA-positive MVs in the plasma is 221 MVs/μL or more, and wherein the method has a ROC sensitivity of 25% or more and a specificity of 100%.

In any one of the methods of the invention described above, the expression of the one or more biomarkers may be detected via PCR, preferably RT-qPCR; or via an immunoassay, preferably an enzyme-linked immunosorbent assay (ELISA); or via a cytometry assay, such as a mass cytometry assay or a flow cytometry assay; or by a mass spectrometry assay; preferably the one or more biomarkers are detected via a flow cytometry assay, preferably using an anti-biomarker antibody conjugated to a fluorophore.

In any one of the methods of the invention described above, the protein and/or the blood cell type may be detected via an immunoassay, preferably an enzyme-linked immunosorbent assay (ELISA); or via a cytometry assay, such as a mass cytometry assay or a flow cytometry assay; or by a mass spectrometry assay; or by spectrophotometry.

In any one of the methods of the invention described above, the compound may be detected via a mass spectrometry assay; or by nuclear magnetic resonance; or by spectrophotometry.

In any one of the methods of the invention described above, MVs may be detected via an immunoassay, preferably an enzyme-linked immunosorbent assay (ELISA); or via a cytometry assay, such as a mass cytometry assay or a flow cytometry assay; or by a mass spectrometry assay; preferably MVs are detected via a flow cytometry assay, preferably using an anti-annexin V antibody conjugated to a fluorophore such as fluorescein isothiocyanate (FITC).

In any one of the methods of the invention described above, the individual may be a mammal, such as a feline mammal, a canine mammal, a porcine mammal, an equine mammal, a bovine mammal, a murine mammal e.g. a mouse or a rat, or a primate mammal e.g. a monkey or chimpanzee, preferably wherein the individual is a human.

The invention also provides a kit comprising reagents for:
a) detecting microvesicles (MVs) and determining the concentration of MVs in an individual's plasma; and
b) detecting the presence of one, more or all biomarkers in a group of biomarkers expressed in MVs, wherein the group of biomarkers consists of CEA, A33, LGR5, EPhB2, ICAM-1, CD31, CD42a, CD31+/CD42a−, CK20, CK7, CK20+/CK7−, HLA-DR or CD147.

The kit of the invention described above may comprise reagents for detecting the presence of the Component Factor 1 (CF1) group of biomarkers.

Any one of the kits of the invention described above may further comprise reagents for detecting the presence of one, more or all of the biomarkers CEA, A33, CK20, CK7, CK20+/CK7−, HLA-DR and CD147

Any one of the kits of the invention described above may comprise reagents for detecting the presence of the Component Factor 1 (CF2) group of biomarkers.

Any one of the kits of the invention described above may further comprise reagents for detecting the presence of one, more or all of the biomarkers ICAM-1, CD31, CD42a, CD31+/CD42a−, CK20, CK7, CK20+/CK7−, HLA-DR and CD147.

Any one of the kits of the invention described above may further comprise reagents for detecting and quantifying in blood:
a) a blood protein, wherein the protein is haemoglobin, albumen, C-reactive protein (CRP) and/or carcinoembryonic antigen (CEA); and/or
b) a blood cell, wherein the cell is a neutrophil and/or a lymphocyte; and/or
c) a blood compound, wherein the compound is urea and/or creatinine.

In any one of the kits of the invention described above, the reagent for detecting MVs and determining the concentration of MVs in an individual's plasma may comprise an anti-Annexin V antibody, optionally conjugated to a fluorophore, preferably fluorescein isothiocyanate (FITC).

In any one of the kits of the invention described above, the reagents for detecting the presence of the one, more or all biomarkers expressed in MVs may comprise respectively an anti-CEA antibody, an anti-A33 antibody, an anti-LGR5 antibody, an anti-EPhB2 antibody, an anti-ICAM-1 antibody, an anti-CD31 antibody, an anti-CD42a antibody, CD31+/CD42a−, an anti-CK20 antibody, an anti-CK7 antibody, CK20+/CK7-, an anti-HLA-DR antibody and an anti-CD147 antibody, preferably wherein the antibodies are labelled with a fluorophore.

In any one of the kits of the invention described above, the reagents for detecting the presence of the one, more or all biomarkers expressed in MVs may comprise:
a) forward and reverse primers for amplifying, in a nucleic acid amplification reaction, nucleic acid encoding the one or more biomarkers;
b) a detectable probe for detecting amplification products generated by the forward and reverse primers in the nucleic acid amplification reaction; and optionally
c) a nucleic acid amplification enzyme, preferably a DNA polymerase

Any one of the kits of the invention described above may comprise a nucleic acid amplification enzyme, preferably a DNA polymerase, and further comprising a reverse transcriptase enzyme.

The invention also provides a chip comprising:
a) a first input channel for introduction of MVs;
b) a second input channel for introduction of reagents for determining the concentration of MVs in an individual's plasma, or determining the concentration of MVs in the individual's plasma which test positive for the detectable surface expression of one or more biomarkers;
c) a third input channel for introduction of wash buffer;
d) a first chamber for mixing the MVs with the reagents;
e) a second chamber for washing the MVs from the reagents using the wash buffer;
f) a first output channel for releasing MVs after mixing and washing, for introduction into a flow cytometer; and
g) a second output channel for releasing the reagents and wash buffer after mixing and washing.

### BRIEF DESCRIPTION OF THE FIGURES

FIG. 1. The plasma level of total microvesicles in study participants. BCRP: benign colorectal polyps, CRC: colorectal cancer. ****: p<0.0001.

FIG. 2. Area under the receiver operator curve (AUC) for the diagnosis of benign colorectal polyps (line in green) and colorectal cancer (line in red) using total plasma microvesicles count (MVs/μL). CI: confidence interval.

FIG. 3. The plasma levels of microvesicles (MVs) positive for colorectal cancer (CRC) markers in healthy controls, CRC and benign colorectal polyps (BCRP): A) carcinoembryonic antigen (CEA); B) A33; C) leucine rich repeat containing G protein coupled receptor 5 (LGR5); D) ephrin type-B receptor 2 (EPHB2); E) intercellular adhesion molecule (ICAM); F) CD31; G) CD42a; and H) CD31+/CD42a−. *: p<0.05. **: p<0.01. ***: p<0.001. ****: p<0.0001.

FIG. 4. Correlation matrix, heatmap showing significance of Spearman's rho. Dendogram showing markers clustering into three groups: A) CD31, total microvesicles (MVs), EPHB2, CD31+/CD42a− and CD42a; B) LGR5, A33, and CEA; C) ICAM. **: p<0.01.

FIG. 5. Scree plot showing 2 component factors appropriateness for further investigation.

FIG. 6. Area under the receiver operator curve (AUC) for the diagnosis of benign colorectal polyps (BCRP, line in green) and colorectal cancer (CRC, line in red) for component factors 1 and 2. CI: confidence interval.

FIG. 7. The levels of blood biomarkers that are statistically significantly different in benign colorectal polyps (BCRP) and colorectal cancer (CRC): A) Haemoglobin; B) Neutrophils; C) Lymphocytes; D) Albumin; E) C-reactive protein; F) Urea; and G) Creatinine. *: p<0.05. **: p<0.01. ***: p<0.001.

FIG. 8. Area under the receiver operator curve (AUC) for routine blood tests and microvesicles positive for CEA for the diagnosis of colorectal cancer.

FIG. 9. Venn diagram of routine blood results that may distinguish benign colorectal polyps from colorectal cancer. CRP: C-reactive protein.

FIG. 10. Identification of MVs using flow cytometry. A-B) 1.1 μm latex beads were used to determine forward scatter gating for MVs. 3 μm latex beads and volume of plasma from which the MVs were analyzed were used to determine the absolute MV count from the number of events captured by the flow cytometer. C) Total MVs were gated using forward scatter for size and annexin V positivity. (FSC=forward scatter; SSC=side scatter; AnV+=annexin V+)

FIG. 11. Total Annexin V+ MVs measured in healthy controls (n=24), patients with polyps (n=16), colorectal cancer (n=31), and other bowel conditions (n=7). A) Scatter plot diagram of total microvesicle count measured from 100 μl of platelet poor plasma obtained from subjects. B) Receiver operated curve analyses depicting sensitivity % of total MVs in detecting colorectal cancer patients from healthy controls, patients with polyps from healthy controls, and colorectal cancer patients from polyp group. C) Receiver operated curve analyses depicting sensitivity % of total MVs in detecting colorectal cancer patients from the other conditions group and patients with polyps from the other conditions group. (Data displayed are median±interquartile range. **P<0.01; ***P<0.001.)

FIG. 12. A33+ MVs measured in healthy controls (n=24), patients with polyps (n=16), colorectal cancer (n=30), and other bowel conditions (n=7). A) Scatter plot diagram of MV count measured from 100 μl of platelet A33+ plasma obtained from subjects. B) Receiver operated curve analyses depicting sensitivity % of A33+ MVs in detecting colorectal cancer patients from healthy controls, patients with polyps from healthy controls, and colorectal cancer patients from polyp group. C) Receiver operated curve analyses depicting sensitivity % of A33+ MVs in detecting colorectal cancer patients from the other conditions group and patients with polyps from the other conditions group. (Data displayed are median±interquartile range. **P<0.01; ****P<0.0001.)

FIG. 13. CEA+ MVs measured in healthy controls (n=24), patients with polyps (n=16), colorectal cancer (n=30), and other bowel conditions (n=7). A) Scatter plot diagram of CEA+ microvesicle count measured from 100p of platelet poor plasma obtained from subjects. B) Receiver operated curve analyses depicting sensitivity % of CEA+ MVs in detecting colorectal cancer patients from healthy controls, patients with polyps from healthy controls, and colorectal cancer patients from polyp group. C) Receiver operated curve analyses depicting sensitivity % of CEA+ MVs in detecting colorectal cancer patients from the other conditions group and patients with polyps from the other conditions group. (Data displayed are median±interquartile range. *P<0.05; **P<0.01; ****P<0.0001.)

FIG. 14. LGR5+ MVs measured in healthy controls (n=23), patients with polyps (n=16), colorectal cancer (n=28), and other bowel conditions (n=7). A) Scatter plot diagram of LGR5+ microvesicle count measured from 100 μl of platelet poor plasma obtained from subjects. B) Receiver operated curve analyses depicting sensitivity % of LGR5+ MVs in detecting colorectal cancer patients from healthy controls, patients with polyps from healthy controls, and colorectal cancer patients from polyp group. C) Receiver operated curve analyses depicting sensitivity % of LGR5+ MVs in detecting colorectal cancer patients from the other conditions group and patients with polyps from the other conditions group. (Data displayed are median±interquartile range. *P<0.05; ***P<0.001; ****P<0.0001.)

FIG. 15. EPhB2+ MVs measured in healthy controls (n=23), patients with polyps (n=16), colorectal cancer (n=28), and other bowel conditions (n=7). A) Scatter plot diagram of EPhB2 microvesicle count measured from 100 μl of platelet poor plasma obtained from subjects. B) Receiver operated curve analyses depicting sensitivity % of EPhB2 MVs in detecting colorectal cancer patients from healthy controls, patients with polyps from healthy controls, and colorectal cancer patients from polyp group. C) Receiver operated curve analyses depicting sensitivity % of EPhB2+ MVs in detecting colorectal cancer patients from the other conditions group and patients with polyps from the other conditions group. (Data displayed are median±interquartile range. *P<0.05; ***P<0.001; ****P<0.0001.)

FIG. 16. Cytokeratin 20 (CK20)+ and Cytokeratin 7 (CK7)+ MVs measured in healthy controls (n=17), patients with polyps (n=16), colorectal cancer (n=27), and other bowel conditions (n=7). A-B) Scatter plot diagram of Cytokeratin 20+ and 7+MV count measured from 100p of platelet poor plasma obtained from subjects. C-D) Receiver operated curve analyses depicting sensitivity % of cytokeratin 20+ and cytokeratin 7+ MVs in detecting colorectal cancer patients from healthy controls, patients with polyps from healthy controls, and colorectal cancer patients from polyp group. (Data displayed are median±interquartile range. **P<0.01; ***P<0.001; ****P<0.0001.)

FIG. 17. Cytokeratin 20+/cytokeratin 7− MVs measured in healthy controls (n=17), patients with polyps (n=16), colorectal cancer (n=27), and other bowel conditions (n=7). A) Scatter plot diagram of CK20+/CK7− MV count measured from 100 μl of platelet poor plasma obtained from subjects. B) Receiver operated curve analyses depicting sensitivity % of CK20+/CK7− MVs in detecting colorectal cancer patients from healthy controls, patients with polyps from healthy controls, and colorectal cancer patients from polyp group. C) Receiver operated curve analyses depicting sensitivity % of CK20+/CK7− MVs in detecting colorectal cancer patients from the other conditions group and patients with polyps from the other conditions group. (Data displayed are median±interquartile range. *P<0.05; **P<0.01)

FIG. 18. HLA-DR+ MVs measured in healthy controls (n=17), patients with polyps (n=16), colorectal cancer (n=25), and other bowel conditions (n=7). A) Scatter plot diagram of HLA-DR+MV count measured from 100 μl of platelet poor plasma obtained from subjects. B) Receiver operated curve analyses depicting sensitivity % of HLA-DR+ MVs in detecting colorectal cancer patients from healthy controls, patients with polyps from healthy controls, and colorectal cancer patients from polyp group. C) Receiver operated curve analyses depicting sensitivity % of HLA-DR+ MVs in detecting colorectal cancer patients from the other conditions group and patients with polyps from the other conditions group. (Data displayed are median±interquartile range. ***P<0.001.)

FIG. 19. CD147+ MVs measured in healthy controls (n=17), patients with polyps (n=16), colorectal cancer (n=25), and other bowel conditions (n=7). A) Scatter plot diagram of CD147+MV count measured from 100p of platelet poor plasma obtained from subjects. B) Receiver operated curve analyses depicting sensitivity % of CD147+ MVs in detecting colorectal cancer patients from healthy controls, patients with polyps from healthy controls, and colorectal cancer patients from polyp group. C) Receiver operated curve analyses depicting sensitivity % of CD147+ MVs in detecting colorectal cancer patients from the other conditions group and patients with polyps from the other conditions group. (Data displayed are median±interquartile range. **P<0.01; ***P<0.001.)

FIG. 20. PECAM1+ MVs measured in healthy controls (n=24), patients with polyps (n=16), colorectal cancer (n=30), and other bowel conditions (n=7). A) Scatter plot diagram of PECAM1+ microvesicle count measured from 100p of platelet poor plasma obtained from subjects. B) Receiver operated curve analyses depicting sensitivity % of PECAM1+ MVs in detecting colorectal cancer patients from healthy controls, patients with polyps from healthy controls, and colorectal cancer patients from polyp group. C) Receiver operated curve analyses depicting sensitivity % of PECAM1+ MVs in detecting colorectal cancer patients from the other conditions group and patients with polyps from the other conditions group. (Data displayed are median±interquartile range. *P<0.05; ****P<0.0001.)

FIG. 21. CD42a+ MVs measured in healthy controls (n=24), patients with polyps (n=16), colorectal cancer (n=30), and other bowel conditions (n=7). A) Scatter plot diagram of CD42a+ MV count measured from 100 μl of platelet poor plasma obtained from subjects. B) Receiver operated curve analyses depicting sensitivity % of CD42a+ MVs in detecting colorectal cancer patients from healthy controls, patients with polyps from healthy controls, and colorectal cancer patients from polyp group. C) Receiver operated curve analyses depicting sensitivity % of CD42a+ MVs in detecting colorectal cancer patients from the other conditions group and patients with polyps from the other conditions group. (Data displayed are median±interquartile range. ***P<0.001; ****P<0.0001.)

FIG. 22. PECAM1+/CD42a− MVs measured in healthy controls (n=24), patients with polyps (n=16), colorectal cancer (n=30), and other bowel conditions (n=7). A) Scatter plot diagram of total microvesicle count measured from 100 μl of platelet poor plasma obtained from subjects. B) Receiver operated curve analyses depicting sensitivity % of total MVs in detecting colorectal cancer patients from healthy controls, patients with polyps from healthy controls, and colorectal cancer patients from polyp group. C) Receiver operated curve analyses depicting sensitivity % of PECAM1+/CD42a− MVs in detecting colorectal cancer patients from the other conditions group and patients with polyps from the other conditions group. (Data displayed are median±interquartile range. **P<0.001; ****P<0.0001.)

FIG. 23. Serum creatinine measured in patients with polyps (n=12) and colorectal cancer (n=20). A) Scatter plot diagram of creatinine obtained from subjects. B) Receiver operated curve analyses depicting sensitivity % of creatinine in detecting colorectal cancer patients from patients with polyps. (Data displayed are median interquartile range. **P<0.01.)

FIG. 24. Serum albumin measured in patients with polyps (n=12) and colorectal cancer (n=20). A) Scatter plot diagram of creatinine obtained from subjects. B) Receiver operated curve analyses depicting sensitivity % of albumin in detecting colorectal cancer patients from patients with polyps. (Data displayed are median interquartile range. ***P<0.001.)

FIG. 25. C-reactive protein (CRP) measured in patients with polyps (n=7) and colorectal cancer (n=18). A) Scatter plot diagram of creatinine obtained from subjects. B) Receiver operated curve analyses depicting sensitivity % of CRP in detecting colorectal cancer patients from patients with polyps. (Data displayed are median interquartile range. **P<0.01.)

FIG. 26. Haemoglobin (g/L) measured in patients with polyps (n=12) and colorectal cancer (n=20). A) Scatter plot diagram of creatinine obtained from subjects. B) Receiver operated curve analyses depicting sensitivity % of CRP in detecting colorectal cancer patients from patients with polyps. (Data displayed are median interquartile range. ***P<0.001.)

FIG. 27. Serum urea (mmol/L) measured in patients with polyps (n=11) and colorectal cancer (n=20). A) Scatter plot diagram of creatinine obtained from subjects. B) Receiver operated curve analyses depicting sensitivity % of urea in detecting colorectal cancer patients from patients with polyps. (Data displayed are median interquartile range. **P<0.01.)

### DETAILED DESCRIPTION

The present invention is concerned with methods of assessing whether an individual has colorectal cancer (CRC) or is at risk of developing CRC, by determining the concentration of microvesicles (MVs) in the individual's plasma and classifying the individual as having CRC or benign colorectal polyps (BRCP), when the concentration of MVs is statistically significantly higher compared to control.

The present invention also relates to methods that further comprise determining the concentration of MVs in the individual's plasma which test positive for the detectable expression, preferably detectable surface expression, of one or more biomarkers, and classifying the individual as having BCRP or CRC when the concentration of biomarker positive MVs in the individual's plasma is statistically significantly higher compared to control and/or when the concentration of biomarker positive MVs in the individual's plasma exceeds a threshold value. The biomarker may be CEA, A33, LGR5, EPhB2, ICAM-1, CD31, CD42a, CD31+/CD42a−, CK20, cytokeratin 7, CK20+/CK7−, HLA-DR and/or CD147.

The present invention also relates to methods that further comprise determining the concentration of components in blood, and distinguishing the individual as having CRC rather than BRCP and/or classifying the individual as having CRC. The blood component may be haemoglobin, neutrophils, lymphocytes, albumin, CRP, urea, creatinine and/or CEA.

### Assessment Methods

The methods of the invention provide means for assessing whether an individual has colorectal cancer (CRC) or is at risk of developing CRC.

By “has” CRC or “is at risk of developing” CRC a skilled person will understand that the methods of the invention represents a diagnostic ‘prediction’ because any assessment conducted in accordance with the invention is unlikely to be capable of diagnosing every individual as having or not having CRC or as being at risk or not at risk of developing CRC with 100% specificity and 100% sensitivity. Threshold values are provided for the various parameters applied by the user for positively predicting the presence of CRC or assessing the risk of developing CRC in an individual. Using such thresholds, the false positive and false negative rates will vary. In other words, the inventors have discovered that the assays of the invention can achieve variable levels of sensitivity and specificity for assessing whether an individual has CRC or is at risk of developing CRC, as defined by receiver operating characteristics (ROC), in accordance with the threshold applied by the user. Such sensitivity and specificity can be seen from the data disclosed herein to be achievable at high proportions, demonstrating accurate and statistically-significant discriminatory capability.

The same factors apply to assessing the individual as having benign colorectal polyps (BCRPs). Having BCRPs or having a likelihood of having BCRPs means that the individual is at risk of developing CRC.

Threshold values described herein have been pre-determined by the inventors to correlate with CRC and/or BCRP, with a high level of statistical accuracy as explained further herein.

Accordingly, any of the methods described or defined herein for assessing whether an individual has CRC or is at risk of developing CRC can alternatively be defined as a method for predicting whether an individual has CRC or is at risk of developing CRC, or a method for classifying whether an individual has CRC or is at risk of developing CRC, or a method for identifying whether an individual has CRC or is at risk of developing CRC.

Any of the methods described or defined herein wherein an individual is classified as having BCRPs can alternatively be defined as a method for predicting whether an individual has BCRPs, or a method for identifying whether an individual has BCRPs, or a method for assessing whether an individual has BCRPs.

Accordingly, the present invention relates to stratification or screening processes for CRC, including methods that further determine whether the individual has CRC or BRCP depending on the concentration of MVs in the individual's plasma and further including methods that determine whether the individual has CRC or BRCP depending on the concentration of MVs in the individual's plasma which test positive for the detectable expression, preferably detectable surface expression, of one or more biomarkers, and/or the concentration of components in the individual's blood.

The invention also provides a variety of assay methods, each comprising a variety of steps which can be performed in any appropriate order, including methods of the following: measuring in a sample; assessing a sample; detecting a sample; analyzing a sample; evaluating a sample; assaying a sample; measuring nucleic acids in a sample; assessing nucleic acids in a sample; detecting nucleic acids in a sample; analyzing nucleic acids in a sample; evaluating nucleic acids in a sample; assaying nucleic acids in a sample; measuring the concentration of MVs in a sample; assessing the concentration of MVs in a sample; detecting the concentration of MVs in a sample; evaluating the concentration of MVs in a sample; assaying the concentration of MVs in a sample; measuring the fold change of MVs in a sample compared to control; assessing the fold change of MVs in a sample compared to control; detecting the fold change of MVs in a sample compared to control; evaluating the fold change of MVs in a sample compared to control; assaying the fold change of MVs in a sample compared to control; measuring the concentration of MVs in a sample which test positive for the detectable expression, optionally detectable surface expression, of one or more biomarkers; assessing the concentration of MVs in a sample which test positive for the detectable expression, optionally detectable surface expression, of one or more biomarkers; detecting the concentration of MVs in a sample which test positive for the detectable expression, optionally detectable surface expression, of one or more biomarkers; evaluating the concentration of MVs in a sample which test positive for the detectable expression, optionally detectable surface expression, of one or more biomarkers; assaying the concentration of MVs in a sample which test positive for the detectable expression, optionally detectable surface expression, of one or more biomarkers; measuring the fold change of MVs which test positive for the detectable expression, optionally detectable surface expression, of one or more biomarkers in a sample compared to control; assessing the fold change of MVs which test positive for the detectable expression, optionally detectable surface expression, of one or more biomarkers in a sample compared to control; detecting the fold change of MVs which test positive for the detectable expression, optionally detectable surface expression, of one or more biomarkers in a sample compared to control; evaluating the fold change of MVs which test positive for the detectable expression, optionally detectable surface expression, of one or more biomarkers in a sample compared to control; assaying the fold change of MVs which test positive for the detectable expression, optionally detectable surface expression, of one or more biomarkers in a sample compared to control.

The sample may be from a tissue from an individual suspected of having, or at risk of having BCRPs or CRC, preferably plasma;

Any of the methods described or defined herein may additionally involve treating an individual for BCRPs or CRC when the individual has been determined to have a threshold concentration of MVs compared to control and/or a threshold concentration of MVs which test positive for the detectable expression, optionally detectable surface expression, of one or more biomarkers in a sample compared to control.

Any of the methods described or defined herein may comprise providing MVs from a sample; performing a capturing step; performing a binding step; performing a purification step; performing a capturing step comprising binding of MVs to binding molecules specific to any biomarker disclosed herein and collecting complexes thereof.

### Individual

In any of the methods of the invention disclosed herein, the term “individual” may be a human. The most preferred individual to which the methods of the invention are applicable are humans.

In any of the methods of the invention disclosed herein the individual may be a non-human animal. For example, methods of the invention disclosed herein may be applied to non-human animals, for example to determine the efficacy of new therapeutics, new therapeutic strategies, new modes of administration of pre-existing therapeutic strategies, or surgical methods. Thus in any of the methods of the invention disclosed herein the individual may be a mammal, such as a feline mammal, a canine mammal, a porcine mammal, an equine mammal, a bovine mammal, a rodent mammal, a murine mammal e.g. a mouse or a rat, or a primate mammal e.g. a monkey or chimpanzee.

### Microvesicles

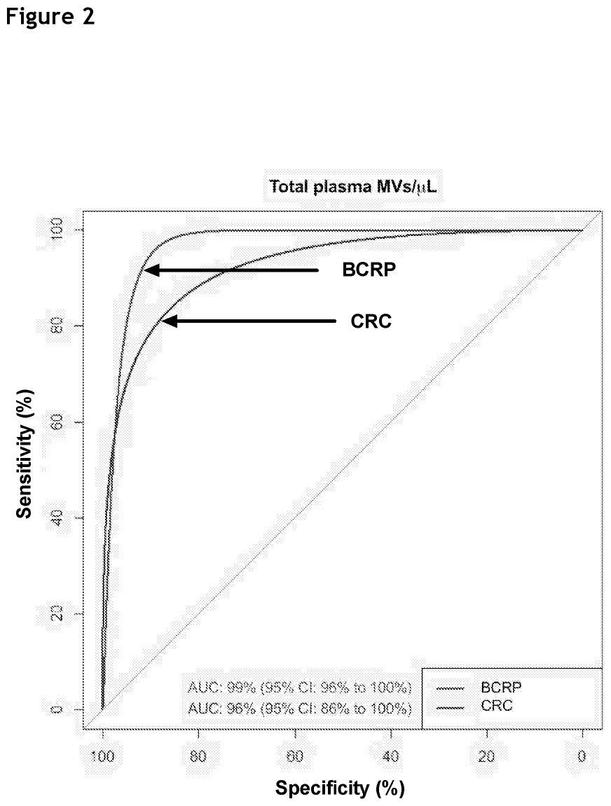

Microvesicles (MVs) are one of three types of extracellular vesicles (EVs) distinguished by their size (˜150-1000 nm) and their biogenesis (see Lawson et al., J Endocrinol. 2016, 228(2), ppR57-71 and in particular FIG. 2, and see also Lacroix et al., J. Thrombosis and Haemostasis, 2012, 11: pp 1190-1193). The other two main types of EVs are exosomes and apoptotic bodies. Exosomes are smaller (<150 nm) and derive from multivesicular bodies within the cell's endosomal system. Apoptotic bodies are larger (>1000 nm) and arise from dying cells. In the context of neoplasia detection of MVs, they may be used as a form of liquid biopsy from tumour cells. Accordingly, MVs are a different biological structure compared to exosomes. MVs and exosomes are distinguishable and can be detected and purified separately based on size and/or using biological markers which are unique to either structure.

Accordingly, in all of the methods of the invention disclosed and defined herein, MVs would not be classified as either exosomes nor apoptotic bodies. Accordingly, in all of the methods of the invention disclosed and defined herein, MVs are not exosomes or apoptotic bodies.

In any of the methods of the invention disclosed and defined herein, MVs may be phosphatidylserine (PS) positive MVs. Accordingly, PS-positive MVs may be identified using detection assays and/or antibodies or binding molecules specific for PS and/or antibodies or binding molecules specific for molecules that bind to PS. For example, Annexin V binds to PS on MVs, and therefore antibodies or other molecules that bind, preferably specifically bind, to Annexin V may be used. Lactadherin also binds to PS on MVs, and therefore antibodies or other molecules that bind, preferably specifically bind, to lactadherin may also be used.

In any of the methods of the invention disclosed and defined herein, MVs may have a size greater than about 0.15 μm but less than about 1.2 μm.

### Purification of MVs

MVs may be processed and purified from a sample obtained from an individual in any way that the user deems appropriate. If the sample is a blood sample, the sample may be processed by centrifugation to obtain plasma, for example platelet poor plasma. In an embodiment, the centrifugation protocol may be to spin down at 5000 g twice for 5 minutes. A MV pellet may be recovered from plasma using further centrifugation. In an aspect, the centrifugation protocol may be to spin down at 17000 g for 1 hour. The MV pellet may then be reconstituted in buffer, for example Annexin V buffer, to obtain a sample of MVs.

### Detection and Quantification of MVs

After obtaining a purified sample of MVs, the concentration of MVs can be determined using routine methods in the art, for example flow cytometry.

In flow cytometry, one or more beams of light, e.g., each of a single wavelength, are directed onto a hydrodynamically-focused stream of fluid. Suspended particles passing through the beams scatter the light, and fluorescent chemicals found in the particles or attached to the particles may be excited. The scattered and/or fluorescent light is analysed by detectors within the device, from which information about the particles and fluorescence can be determined. In the context of the present invention, flow cytometry can be used to count cells and detect biomarkers, for example.

In the methods of the present invention, if using flow cytometry, antibodies conjugated to a label which are in a suitable buffer can be used to detect MVs. For example, an anti-annexin V antibody can be conjugated to fluorescein isothiocyanate (FITC) and diluted in annexin V buffer, for use in flow cytometry. Beads, for example 1.1 μm latex beads, can be used to set the upper threshold on forward scatter to distinguish maximum MV size.

Beads, for example 3 μm latex beads, and volume of plasma from which the MVs were analyzed can be used to quantify MV count from the number of events captured by the flow cytometer. Specifically, the concentration of MVs can be determined by using the proportion of a fixed number of 3 m latex beads counted and the quantity of the supernatant or plasma from which the MVs were analysed.

### Source of Microvesicles

In any of the methods of the invention disclosed herein, the sample of microvesicles (MVs) which have been obtained from the individual is preferably obtained from the plasma of the individual. Alternatively, the sample of MVs which have been obtained from the individual may be obtained from whole blood, a blood fraction, serum, ascitic fluid or urine of the individual.

### Thresholds for Analysis and Controls

In any of the methods of the invention that determine the concentration of MVs as disclosed herein and/or that determine the concentration of biomarker-positive MVs as disclosed herein and/or that determine the concentration of blood components as disclosed herein, the term “threshold” describes the concentration of MVs and/or biomarker-positive MVs and/or blood components above or below which an individual is classified as having BRCPs or CRC.

The threshold value may be associated with ROC values, in which case the threshold describes the concentration of MVs and/or biomarker-positive MVs and/or blood components above or below which an individual is classified as having BRCPs or CRC at certain sensitivity and specificity values.

The threshold used may favour a high sensitivity, corresponding to a high negative predictive value. Such a threshold is preferably used for the methods of the invention that determine the concentration of MVs as disclosed herein and/or the concentration of biomarker-positive MVs. In this case, for individuals that do not fall within the criteria set by the threshold value, there is a high probability that they truly do not have BRCP or CRC. As such, the method of determining the level of MVs may act as a “rule out” test. However, the threshold may also have high specificity, in which case the method of determining the concentration of MVs and/or biomarker-positive MVs and/or blood components may have exceptional discrimination, leading to accurate determination of whether an individual has BRCPs or CRC, or not.

The threshold used may favour high specificity, corresponding to a high positive predictive value. Such a threshold is preferably used for the methods of the invention that determine the concentration of components in the individual's blood, as further described and defined herein. In this case, for individuals that fall within the criteria set by the threshold value, the test will distinguish with high probability the individual as having CRC, or as having CRC rather than BRCP.

In any of the methods of the invention disclosed and defined herein the concentration of MVs in the individual's plasma is statistically significantly higher compared to control, i.e. the concentration of MVs in the plasma of a control individual or individuals.

In any of the methods of the invention disclosed and defined herein the concentration of MVs in the plasma of a control may be the mean concentration of MVs in the plasma of a population of control individuals.

In any of the methods of the invention disclosed and defined herein of assessing whether an individual has CRC, or is at risk of developing CRC, the control individual or individuals is a healthy control individual or healthy control individuals who do not have CRC or BCRP.

In any of the methods of the invention disclosed and defined herein of assessing whether an individual has CRC, or is at risk of developing CRC, the control individual or individuals may be an individual or individuals who have BCRP.

In any of the methods of the invention disclosed and defined herein of assessing whether an individual has BCRP, or is at risk of developing BCRP, the control individual or individuals is a healthy control individual or healthy control individuals who do not have CRC or BCRP.

Methods of the invention disclosed and defined herein involve determining the concentration of MVs in the individual's plasma as well as methods for determining the concentration of MVs in the individual's plasma which test positive for the detectable expression of one or more biomarkers. Where certain threshold values of numbers of MVs are stated, the skilled person will appreciate that ROC analysis may be performed so as to define alternative threshold values of numbers of MVs which correspond to any relevant combination of ROC sensitivity and specificity. The precise numbers may therefore be optimised for detection in a given system, when using a given set of specific reagents.

Where the concentration of MVs is stated as a threshold value of numbers of MVs which is the mean value, such as in the present claims and embodiments set out herein, the mean value can alternatively be expressed as the median value together with confidence limits.

### Statistical Analysis and Metrics

Any of the methods of the invention disclosed herein may be performed by assays which provide a high degree of robustness in terms of statistical discrimination. Thus any of the methods of the invention disclosed herein may be performed by assays which are characterised by a variety of alternative statistical parameters. Preferred statistical parameters which may characterise methods of the invention include parameters based on receiver operating characteristics (ROC) analysis or parameters relating to statistical significance at particular confidence levels.

### Statistical Significance

Methods of the invention involve classifying the individual as having BCRPs or CRC when the concentration of MVs and/or concentration of biomarker positive MVs in the individual's plasma is statistically significantly higher compared to control.

As known in the art, a statistically significant difference is one that a user can determine, with a certain amount of confidence, as being real and not as a result of random chance. The confidence can be derived from using a p-value, which is the probability that an observed difference could have occurred by chance. For example, if a difference is observed with a p-value of 0.05, then there is a 5% probability that the difference has occurred by random chance. In other words, there is a 95% chance that the difference is real.

In the context of the methods of the invention, if the concentration of MVs in the individual's plasma is statistically significantly higher compared to control with a p-value of 0.05, then the probability of that difference being real is 95%. In the methods of the invention, the highest p-value used is 0.05. Preferably the value of p is p≤0.05, p≤0.01, p≤0.001 or p≤0.0001.

In the methods of the invention, the statistical test used can be one that the user deems appropriate. For example, differences in mean can be assessed using the two way t-test and Mann Whitney U test. A two-way ANOVA test can be used to examine the difference in mean of groups of more than two. The method by which the statistical test is done is not essential, provided that the user can arrive at a determination that the concentration of MVs and/or concentration of biomarker positive MVs in the individual's plasma is statistically significantly higher compared to control. The term “control” is defined further herein.

### Receiver Operating Characteristics

Methods of the invention may be defined by statistical parameters based on receiver operating characteristics (ROC).

A ROC curve plots the true positive rate (sensitivity) against the false positive rate (1—specificity) at various threshold settings. The sensitivity and specificity are measures of the statistical robustness of the assay. For example, a sensitivity of 100% means that all patients with a given disease/condition will be identified using the assay. A specificity of 100% means that all patients who do not have a given disease/condition will be identified as such.

The methods of the present invention demonstrate high levels of sensitivity and specificity in order to determine whether an individual has CRC or is at risk of developing CRC.

ROC analysis also allows determination of the area under the curve (AUC), which indicates the accuracy of a diagnostic test i.e. the ability to diagnose patients with and without the disease or condition based on the test. A hypothetical test that has been assessed with ROC and has an AUC of 0.5 indicates that the test has a 50% chance of identifying an individual with a given disease/condition. An AUC of more than 0.5 is considered to have a reasonable discriminating ability to diagnose individuals with and without a given disease/condition.

The term “about” is to be understood as providing a range of +/−5% of the value.

### Thresholds and Statistical Parameters for Plasma Concentration of MVs

In the methods of the invention disclosed herein, in the assay which is performed to identify an individual as having BCRPs or CRC, the plasma concentration of MVs may be 144 MVs/μL or more, wherein the method may be characterised as having a sensitivity of 100% and a specificity of 59% as determined by receiver operating characteristics (ROC).

In the methods of the invention disclosed herein, in the assay which is performed to identify an individual as having BCRPs or CRC, the plasma concentration of MVs may be 244 MVs/μL or more, wherein the method may be characterised as having a sensitivity of 100% and a specificity of 93% as determined by receiver operating characteristics (ROC). The assay may be characterised as having an AUC of 0.99 or more. The assay may be characterised as having an AUC of 0.99 or more and wherein the 95% confidence intervals are 0.96-1.00. In any of these methods the p-value may be ≤0.0001.

In any of the methods of the invention disclosed and defined herein the plasma concentration of MVs in the control may be 124 MVs/μL or less.

In any of the methods of the invention disclosed and defined herein the mean plasma concentration of MVs in the control may be 124 MVs/μL or less.

In any of the methods of the invention disclosed and defined herein the plasma concentration, optionally mean plasma concentration, optionally median plasma concentration, of MVs in the control may be 124 MVs/μL or less, or 130 MVs/μL or less, or 140 MVs/μL or less, or 150 MVs/μL or less, or 160 MVs/μL or less, or 170 MVs/μL or less, or 180 MVs/μL or less, or 190 MVs/μL or less, or 200 MVs/μL or less, 200 MVs/μL or less, 210 MVs/μL or less, 220 MVs/μL or less, 230 MVs/μL or less, 240 MVs/μL or less, 250 MVs/μL or less, 260 MVs/μL or less, 270 MVs/μL or less, 280 MVs/μL or less, 290 MVs/μL or less, 300 MVs/μL or less, 310 MVs/μL or less, 320 MVs/μL or less, 330 MVs/μL or less, 340 MVs/μL or less, 350 MVs/μL or less, 360 MVs/μL or less, 370 MVs/μL or less, 380 MVs/μL or less, 390 MVs/μL or less, 400 MVs/μL or less, 410 MVs/μL or less, 420 MVs/μL or less, 430 MVs/μL or less, 440 MVs/μL or less, 450 MVs/μL or less, 460 MVs/μL or less, 470 MVs/μL or less, 480 MVs/μL or less, 490 MVs/μL or less or 500 MVs/μL or less.

In any of the methods of the invention disclosed and defined herein, rather than expressing the plasma concentration, optionally mean plasma concentration, optionally median plasma concentration, of MVs in the control by a particular number of MVs/μL, such as 124 MVs/μL or less or 130 MVs/μL or less as described above, the step of classifying the individual as having BCRPs or CRC may alternatively be achieved when the concentration of MVs in the individual's plasma shows a statistically significant fold change compared to control. Accordingly, in any of the methods of the invention disclosed and defined herein, the individual may be identified as having BCRPs or CRC when the plasma concentration of MVs is 2-fold or more above the plasma concentration of MVs in the control. The individual may be identified as having BCRPs or CRC when the plasma concentration of MVs is 2-fold or more, 2.5-fold or more, 3-fold or more, 3.5-fold or more, 4-fold or more, 4.5-fold or more, 5-fold or more, 5.5-fold or more, 6-fold or more, 6.5-fold or more, 7-fold or more, 7.5-fold or more, 8-fold or more, 8.5-fold or more, 9-fold or more, 9.5-fold or more, 10-fold or more, 10.5-fold or more, 11-fold or more, 11.5-fold or more, 12-fold or more, 12.5-fold or more, 13-fold or more, 13.5-fold or more, 14-fold or more, 14.5-fold or more, 15-fold or more, 15.5-fold or more, 16-fold or more, 16.5-fold or more, 17-fold or more, 17.5-fold or more, 18-fold or more, 18.5-fold or more, 19-fold or more, 19.5-fold or more or 20-fold or more above the plasma concentration of MVs in the control.

### Thresholds and Statistical Parameters for Plasma Concentration of Biomarker-Positive MVs

In the methods of the invention disclosed herein, assays can be performed to identify an individual as having BCRPs or CRC by determining the concentration of MVs in the individual's plasma which test positive for the detectable expression, preferably detectable surface expression, of one or more biomarkers.

The full designations of the biomarkers which may be analysed are as follows: CEA (carcinoembryonic antigen); A33 (cell surface A33 antigen encoded by the GPA33 gene); LGR5 (Leucine-rich repeat-containing G-protein coupled receptor 5, also known as G-protein coupled receptor 49 (GPR49) or G-protein coupled receptor 67 (GPR67)); EPhB2 (Ephrin type-B receptor 2 encoded by the EPHB2 gene); ICAM-1 (Intercellular Adhesion Molecule 1, also known as Cluster of Differentiation 54 or CD54); CD31 (Cluster of Differentiation 31 (CD31), also known as Platelet Endothelial Cell Adhesion Molecule (PECAM-1)); CD42a (Cluster of Differentiation 42a, also known as Glycoprotein IX (platelet) (GP9)); CD31+/CD42a− (endothelial apoptosis marker); CK20 (Cytokeratin 20, also known as Keratin 20, encoded by the KRT20 gene); CK-7 (Keratin, type II cytoskeletal 7, also known as cytokeratin-7, or keratin-7 (K7) or sarcolectin (SCL), a protein encoded by the KRT7 gene); HLA-DR (Human Leukocyte Antigen-DR isotype, an MHC class II cell surface receptor); CD147 (Cluster of Differentiation 147, also known as Basigin (BSG) or extracellular matrix metalloproteinase inducer (EMMPRIN), a protein encoded by the BSG gene.

Where the one or more biomarkers comprises CEA, the individual may be classified as having CRC when the concentration of MVs which test positive for the detectable expression of CEA exceeds a threshold value and wherein the method has a ROC sensitivity of 100% and a specificity of 60% or more, preferably wherein the threshold value is 10 MVs/μL or more. The method may also be characterised as having an AUC of 0.96 or more. The method may also be characterised as having an AUC of 0.96 or more and wherein the 95% confidence intervals are 0.90-1.00. In any of these methods the p-value may be ≤0.0001.

Where the one or more biomarkers comprises A33, the individual may be classified as having CRC when the concentration of MVs which test positive for the detectable expression of A33 exceeds a threshold value and wherein the method has a ROC sensitivity of 100% and a specificity of 13% or more, preferably wherein the threshold value is 13 MVs/μL or more. The method may also be characterised as having an AUC of 0.87 or more. The method may also be characterised as having an AUC of 0.87 or more and wherein the 95% confidence intervals are 0.75-0.99. In any of these methods the p-value may be ≤0.0001.

Where the one or more biomarkers comprises LGR5, the individual may be classified as having CRC when the concentration of MVs which test positive for the detectable expression of LGR5 exceeds a threshold value and wherein the method has a ROC sensitivity of 96% or more and a specificity of 53% or more, preferably wherein the threshold value is 19 MVs/μL or more. The method may also be characterised as having an AUC of 0.87 or more. The method may also be characterised as having an AUC of 0.87 or more and wherein the 95% confidence intervals are 0.76-0.98. In any of these methods the p-value may be ≤0.0001. Where the one or more biomarkers comprises EPhB2, the individual may be classified as having CRC when the concentration of MVs which test positive for the detectable expression of EPhB2 exceeds a threshold value and wherein the method has a ROC sensitivity of 100% and a specificity of 13% or more, preferably wherein the threshold value is 52 MVs/μL or more. The method may also be characterised as having an AUC of 0.84 or more. The method may also be characterised as having an AUC of 0.84 or more and wherein the 95% confidence intervals are 0.72-0.97. In any of these methods the p-value may be ≤0.0001.

Where the one or more biomarkers comprises ICAM-1, the individual may be classified as having CRC when the concentration of MVs which test positive for the detectable expression of ICAM-1 exceeds a threshold value and wherein the method has a ROC sensitivity of 96% or more and a specificity of 47% or more, preferably wherein the threshold value is 12 MVs/μL or more. The method may also be characterised as having an AUC of 0.90 or more. The method may also be characterised as having an AUC of 0.90 or more and wherein the 95% confidence intervals are 0.80-1.00. In any of these methods the p-value may be ≤0.0001.

Where the one or more biomarkers comprises CD31, the individual may be classified as having CRC when the concentration of MVs which test positive for the detectable expression of CD31 exceeds a threshold value and wherein the method has a ROC sensitivity of 100% and a specificity of 60% or more, preferably wherein the threshold value is 38 MVs/μL or more. The method may also be characterised as having an AUC of 0.97 or more. The method may also be characterised as having an AUC of 0.97 or more and wherein the 95% confidence intervals are 0.93-1.00. In any of these methods the p-value may be ≤0.0001.

Where the one or more biomarkers comprises CD42a, the individual may be classified as having CRC when the concentration of MVs which test positive for the detectable expression of CD42a exceeds a threshold value and wherein the method has a ROC sensitivity of 100% and a specificity of 20% or more, preferably wherein the threshold value is 9 MVs/μL or more. The method may also be characterised as having an AUC of 0.86 or more. The method may also be characterised as having an AUC of 0.86 or more and wherein the 95% confidence intervals are 0.75-0.98. In any of these methods the p-value may be ≤0.0001.

Where the one or more biomarkers comprises CD31+/CD42a−, the individual may be classified as having CRC when the concentration of MVs which test positive for the detectable expression of CD31+/CD42a− exceeds a threshold value and wherein the method has a ROC sensitivity of 96% or more and a specificity of 87% or more, preferably wherein the threshold value is 38 MVs/μL or more. The method may also be characterised as having an AUC of 0.95 or more. The method may also be characterised as having an AUC of 0.95 or more and wherein the 95% confidence intervals are 0.87-1.00. In any of these methods the p-value may be ≤0.0001.

Where the one or more biomarkers comprises CK20, the individual may be classified as having CRC when the concentration of MVs which test positive for the detectable expression of CK20 exceeds a threshold value and wherein the method has a ROC sensitivity of 100% and a specificity of 47% or more, preferably wherein the threshold value is 8 MVs/μL or more. The method may also be characterised as having an AUC of 0.76 or more. The method may also be characterised as having an AUC of 0.76 or more and wherein the 95% confidence intervals are 0.61-0.92. In any of these methods the p-value may be ≤0.006.

Where the one or more biomarkers comprises CK7, the individual may be classified as having CRC when the concentration of MVs which test positive for the detectable expression of cytokeratin 7 exceeds a threshold value and wherein the method has a ROC sensitivity of 100% and a specificity of 47% or more, preferably wherein the threshold value is 16 MVs/μL or more. The method may also be characterised as having an AUC of 0.93 or more. The method may also be characterised as having an AUC of 0.93 or more and wherein the 95% confidence intervals are 0.85-1.00. In any of these methods the p-value may be ≤0.0001.

Where the one or more biomarkers comprises CK20+/CK7−, the individual may be classified as having CRC when the concentration of MVs which test positive for the detectable expression of CK20+/CK7− exceeds a threshold value and wherein the method has a ROC sensitivity of 100% and a specificity of 33% or more, preferably wherein the threshold value is 4 MVs/μL or more. The method may also be characterised as having an AUC of 0.75 or more. The method may also be characterised as having an AUC of 0.75 or more and wherein the 95% confidence intervals are 0.60-0.91. In any of these methods the p-value may be ≤0.009.

Where the one or more biomarkers comprises HLA-DR, the individual may be classified as having CRC when the concentration of MVs which test positive for the detectable expression of CK20+/CK7− exceeds a threshold value and wherein the method has a ROC sensitivity of 100% and a specificity of 27% or more, preferably wherein the threshold value is 26 MVs/μL or more. The method may also be characterised as having an AUC of 0.89 or more. The method may also be characterised as having an AUC of 0.89 or more and wherein the 95% confidence intervals are 0.80-0.99. In any of these methods the p-value may be ≤0.0001.

Where the one or more biomarkers comprises CD147, the individual may be classified as having CRC when the concentration of MVs which test positive for the detectable expression of CD147 exceeds a threshold value and wherein the method has a ROC sensitivity of 96% or more and a specificity of 27% or more, preferably wherein the threshold value is 17 MVs/μL or more. The method may also be characterised as having an AUC of 0.85 or more. The method may also be characterised as having an AUC of 0.85 or more and wherein the 95% confidence intervals are 0.73-0.97. In any of these methods the p-value may be ≤0.0001.

Where the one or more biomarkers comprises CEA, the individual may be classified as having BRCP when the concentration of MVs which test positive for the detectable expression of CEA exceeds a threshold value and wherein the method has a ROC sensitivity of 100% and a specificity of 40% or more, preferably wherein the threshold value is 6.37 MVs/μL or more. The method may also be characterised as having an AUC of 0.84 or more. The method may also be characterised as having an AUC of 0.84 or more and wherein the 95% confidence intervals are 0.71-0.98. In any of these methods the p-value may be ≤0.001.

Where the one or more biomarkers comprises LGR5, the individual may be classified as having BRCP when the concentration of MVs which test positive for the detectable expression of LGR5 exceeds a threshold value and wherein the method has a ROC sensitivity of 100% and a specificity of 60% or more, preferably wherein the threshold value is 28.4 MVs/μL or more. The method may also be characterised as having an AUC of 0.93 or more. The method may also be characterised as having an AUC of 0.93 or more and wherein the 95% confidence intervals are 0.85-1.00. In any of these methods the p-value may be ≤0.0001.

Where the one or more biomarkers comprises EPhB2, the individual may be classified as having BRCP when the concentration of MVs which test positive for the detectable expression of EPhB2 exceeds a threshold value and wherein the method has a ROC sensitivity of 100% and a specificity of 47% or more, preferably wherein the threshold value is 1.07 MVs/μL or more. The method may also be characterised as having an AUC of 0.89 or more. The method may also be characterised as having an AUC of 0.89 or more and wherein the 95% confidence intervals are 0.78-1.00. In any of these methods the p-value may be ≤0.0001.

Where the one or more biomarkers comprises ICAM-1, the individual may be classified as having BRCP when the concentration of MVs which test positive for the detectable expression of ICAM-1 exceeds a threshold value and wherein the method has a ROC sensitivity of 100% and a specificity of 13% or more, preferably wherein the threshold value is 3.6 MVs/μL or more. The method may also be characterised as having an AUC of 0.79 or more. The method may also be characterised as having an AUC of 0.79 or more and wherein the 95% confidence intervals are 0.63-0.96. In any of these methods the p-value may be ≤0.006.

Where the one or more biomarkers comprises CD31, the individual may be classified as having BRCP when the concentration of MVs which test positive for the detectable expression of CD31 exceeds a threshold value and wherein the method has a ROC sensitivity of 100% and a specificity of 87% or more, preferably wherein the threshold value is 72 MVs/μL or more. The method may also be characterised as having an AUC of 0.99 or more. The method may also be characterised as having an AUC of 0.99 or more and wherein the 95% confidence intervals are 0.97-1.00. In any of these methods the p-value may be ≤0.0001.

Where the one or more biomarkers comprises CD42a, the individual may be classified as having BRCP when the concentration of MVs which test positive for the detectable expression of CD42a exceeds a threshold value and wherein the method has a ROC sensitivity of 100% and a specificity of 47% or more, preferably wherein the threshold value is 25 MVs/μL or more. The method may also be characterised as having an AUC of 0.90 or more. The method may also be characterised as having an AUC of 0.90 or more and wherein the 95% confidence intervals are 0.79-1.00. In any of these methods the p-value may be ≤0.0001.

Where the one or more biomarkers comprises CD31+/CD42a−, the individual may be classified as having BRCP when the concentration of MVs which test positive for the detectable expression of CD31+/CD42a− exceeds a threshold value and wherein the method has a ROC sensitivity of 100% and a specificity of 87% or more, preferably wherein the threshold value is 44 MVs/μL or more. The method may also be characterised as having an AUC of 0.98 or more. The method may also be characterised as having an AUC of 0.98 or more and wherein the 95% confidence intervals are 0.95-1.00. In any of these methods the p-value may be ≤0.0001.

Where the one or more biomarkers comprises CK7, the individual may be classified as having BRCP when the concentration of MVs which test positive for the detectable expression of cytokeratin 7 exceeds a threshold value and wherein the method has a ROC sensitivity of 100% and a specificity of 33% or more, preferably wherein the threshold value is 11 MVs/μL or more. The method may also be characterised as having an AUC of 0.90 or more. The method may also be characterised as having an AUC of 0.90 or more and wherein the 95% confidence intervals are 0.78-1.00. In any of these methods the p-value may be ≤0.0001.

Where the one or more biomarkers comprises HLA-DR, the individual may be classified as having BRCP when the concentration of MVs which test positive for the detectable expression of HLA-DR exceeds a threshold value and wherein the method has a ROC sensitivity of 100% and a specificity of 7% or more, preferably wherein the threshold value is 17 MVs/μL or more. The method may also be characterised as having an AUC of 0.84 or more. The method may also be characterised as having an AUC of 0.84 or more and wherein the 95% confidence intervals are 0.68-0.99. In any of these methods the p-value may be ≤0.001.

Where the one or more biomarkers comprises CD147, the individual may be classified as having BRCP when the concentration of MVs which test positive for the detectable expression of CD147 exceeds a threshold value and wherein the method has a ROC sensitivity of 100% and a specificity of 13% or more, preferably wherein the threshold value is 7.4 MVs/μL or more. The method may also be characterised as having an AUC of 0.84 or more. The method may also be characterised as having an AUC of 0.84 or more and wherein the 95% confidence intervals are 0.70-0.99. In any of these methods the p-value may be ≤0.0001.

Where the one or more biomarkers comprises the Component Factor 2 (CF2) group of biomarkers, the individual may be classified as having BRCP when the concentration of MVs which test positive for the detectable expression of CF2 biomarkers exceeds a threshold value and wherein the method has a ROC sensitivity of 100% and a specificity of 63% or more, preferably wherein the threshold value is 222 MVs/μL or more. The method may also be characterised as having an AUC of 0.94 or more. The method may also be characterised as having an AUC of 0.94 or more and wherein the 95% confidence intervals are 0.86-1.00. In any of these methods the p-value may be ≤0.001.

Where the one or more biomarkers comprises the Component Factor 1 (CF1) group of biomarkers, the individual may be classified as having BRCP when the concentration of MVs which test positive for the detectable expression of CF1 biomarkers exceeds a threshold value and wherein the method has a ROC sensitivity of 100% and a specificity of 100%, preferably wherein the threshold value is 761 MVs/μL or more. The method may also be characterised as having an AUC of 1.00 or more. The method may also be characterised as having an AUC of 1.00 or more and wherein the 95% confidence intervals are 1.00-1.00. In any of these methods the p-value may be ≤0.0001.

Where the one or more biomarkers comprises the Component Factor 2 (CF2) group of biomarkers, the individual may be classified as having CRC when the concentration of MVs which test positive for the detectable expression of CF2 biomarkers exceeds a threshold value and wherein the method has a ROC sensitivity of 100% and a specificity of 50% or more, preferably wherein the threshold value is 157 MVs/μL or more. The method may also be characterised as having an AUC of 0.93 or more. The method may also be characterised as having an AUC of 0.93 or more and wherein the 95% confidence intervals are 0.84-1.00. In any of these methods the p-value may be ≤0.0001.

Where the one or more biomarkers comprises the Component Factor 1 (CF1) group of biomarkers, the individual may be classified as having CRC when the concentration of MVs which test positive for the detectable expression of CF1 biomarkers exceeds a threshold value and wherein the method has a ROC sensitivity of 100% and a specificity of 56% or more, preferably wherein the threshold value is 439 MVs/μL or more. The method may also be characterised as having an AUC of 0.95 or more. The method may also be characterised as having an AUC of 0.95 or more and wherein the 95% confidence intervals are 0.88-1.00. In any of these methods the p-value may be ≤0.0001.

In any of the above-described methods, the method may be characterised as having an ROC AUC of 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more, 0.90 or more, 0.91 or more, 0.92 or more, 0.93 or more, 0.94 or more, 0.95 or more, 0.96 or more, 0.97 or more, 0.98 or more, 0.99 or 1.00.

In any of the methods of the invention disclosed and defined herein which involve determining the concentration of MVs in the individual's plasma which test positive for the detectable surface expression of one or more biomarkers, rather than expressing the plasma concentration, optionally mean plasma concentration, optionally median plasma concentration, of MVs in the control by a particular number of MVs/μL, the step of classifying the individual as having BCRPs or CRC may alternatively be achieved when the concentration of MVs in the individual's plasma which test positive for the detectable surface expression of one or more biomarkers shows a statistically significant fold change compared to control. Accordingly, in any of the methods of the invention disclosed and defined herein, the individual may be identified as having BCRPs or CRC when the plasma concentration of MVs which test positive for the detectable surface expression of any one or more biomarkers is 2-fold or more above the plasma concentration of MVs which test positive for the detectable surface expression of the biomarker(s) in the control. The individual may be identified as having BCRPs or CRC when the plasma concentration of such MVs is 2-fold or more, 2.5-fold or more, 3-fold or more, 3.5-fold or more, 4-fold or more, 4.5-fold or more, 5-fold or more, 5.5-fold or more, 6-fold or more, 6.5-fold or more, 7-fold or more, 7.5-fold or more, 8-fold or more, 8.5-fold or more, 9-fold or more, 9.5-fold or more, 10-fold or more, 10.5-fold or more, 11-fold or more, 11.5-fold or more, 12-fold or more, 12.5-fold or more, 13-fold or more, 13.5-fold or more, 14-fold or more, 14.5-fold or more, 15-fold or more, 15.5-fold or more, 16-fold or more, 16.5-fold or more, 17-fold or more, 17.5-fold or more, 18-fold or more, 18.5-fold or more, 19-fold or more, 19.5-fold or more or 20-fold or more above the plasma concentration of MVs which test positive for the detectable surface expression of the biomarker(s) in the control.

### Thresholds and Statistical Parameters for Blood Components

In the methods of the invention disclosed herein, assays can be performed to identify an individual as having BCRPs or CRC by determining the concentration of blood components in the individual's blood, whereby the assays comprise providing a sample of blood which has been obtained from the individual,

Where the blood component comprises a protein, the individual may be classified as having CRC when the concentration of the protein is below a threshold value, wherein the protein is haemoglobin, and wherein the method has a ROC sensitivity of 68% or more and a specificity of 100%. The individual may be distinguished as having CRC rather than BCRPs when the concentration of haemoglobin is 133 g/L or less, and wherein the method has a ROC sensitivity of 58.3% or more and a specificity of 90% or more; or wherein the individual is classified as having CRC when the concentration of haemoglobin is 124.5 g/L or less, and wherein the method has a ROC sensitivity of 68% or more and a specificity of 100%. The method may also be characterised as having an AUC of 0.87 or more. The method may also be characterised as having an AUC of 0.87 or more and wherein the 95% confidence intervals are 0.73-1.00. In any of these methods the p-value may be ≤0.001.

Where the blood component comprises a blood cell type, the individual may be classified as having CRC when the concentration of the blood cell type is above a threshold value, wherein the blood cell type is neutrophils, and wherein the method has a ROC sensitivity of 47% or more and a specificity of 100%. The individual may be classified as having CRC when the concentration of neutrophils is 7.7×10⁹/L or more, and wherein the method has a ROC sensitivity of 47% or more and a specificity of 100%. The method may also be characterised as having an AUC of 0.76 or more. The method may also be characterised as having an AUC of 0.76 or more and wherein the 95% confidence intervals are 0.59-0.93. In any of these methods the p-value may be ≤0.02.

Where the blood component comprises a blood cell type, the individual may be classified as having CRC when the concentration of the blood cell type is below a threshold value, wherein the blood cell type is lymphocytes, and wherein the method has a ROC sensitivity of 47% or more and a specificity of 100%. The individual may be classified as having CRC when the concentration of lymphocytes is 1.16×10⁹/L or less, and wherein the method has a ROC sensitivity of 47% or more and a specificity of 100%. The method may also be characterised as having an AUC of 0.81 or more. The method may also be characterised as having an AUC of 0.81 or more and wherein the 95% confidence intervals are 0.64-0.97. In any of these methods the p-value may be ≤0.008.

Where the blood component comprises a protein, the individual may be classified as having CRC when the concentration of the protein is below a threshold value, wherein the protein is albumen, and wherein the method has a ROC sensitivity of 63% or more and a specificity of 100%. The individual may be distinguished as having CRC rather than BCRPs when the concentration of albumen is 43.5 g/L or less, and wherein the method has a ROC sensitivity of 76.9% or more and a specificity of 90% or more; or wherein the individual is classified as having CRC when the concentration of albumen is 39.5 g/L or less, and wherein the method has a ROC sensitivity of 63% or more and a specificity of 100%. The method may also be characterised as having an AUC of 0.90 or more. The method may also be characterised as having an AUC of 0.90 or more and wherein the 95% confidence intervals are 0.78-1.01. In any of these methods the p-value may be ≤0.0004.

Where the blood component comprises a protein, the individual may be classified as having CRC when the concentration of the protein is above a threshold value, wherein the protein is C-reactive protein (CRP), and wherein the method has a ROC sensitivity of 63% or more and a specificity of 100%. The individual may be distinguished as having CRC rather than BCRPs when the concentration of CRP is 19.65 mg/L or more, and wherein the method has a ROC sensitivity of 85.7% or more and a specificity of 90% or more; or wherein the individual is classified as having CRC when the concentration of CRP is 72.75 mg/L or more, and wherein the method has a ROC sensitivity of 63% or more and a specificity of 100%. The method may also be characterised as having an AUC of 0.85 or more. The method may also be characterised as having an AUC of 0.85 or more and wherein the 95% confidence intervals are 0.68-1.03. In any of these methods the p-value may be ≤0.01.

Where the blood component comprises a compound, the individual may be classified as having CRC when the concentration of the compound is below a threshold value, wherein the compound is urea, and wherein the method has a ROC sensitivity of 58% or more and a specificity of 100%. The individual may be distinguished as having CRC rather than BCRPs when the concentration of urea is 5.5 mmol/L or less, and wherein the method has a ROC sensitivity of 54.55% or more and a specificity of 90% or more; or wherein the individual is classified as having CRC when the concentration of urea is 3.85 mmol/L or less, and wherein the method has a ROC sensitivity of 58% or more and a specificity of 100%. The method may also be characterised as having an AUC of 0.85 or more. The method may also be characterised as having an AUC of 0.85 or more and wherein the 95% confidence intervals are 0.71-1.00. In any of these methods the p-value may be ≤0.002.

Where the blood component comprises a compound, the individual may be classified as having CRC when the concentration of the compound is below a threshold value, wherein the compound is creatinine, and wherein the method has a ROC sensitivity of 53% or more and a specificity of 100%. The individual may be distinguished as having CRC rather than BCRPs when the concentration of creatinine is 82.5 mmol/L or less, and wherein the method has a ROC sensitivity of 75% or more and a specificity of 90% or more; or wherein the individual is classified as having CRC when the concentration of creatinine is 63.5 μmol/L or less, and wherein the method has a ROC sensitivity of 53% or more and a specificity of 100%. The method may also be characterised as having an AUC of 0.75 or more. The method may also be characterised as having an AUC of 0.75 or more and wherein the 95% confidence intervals are 0.69-0.98. In any of these methods the p-value may be ≤0.003.

Also described is an assay where the individual may be classified as having CRC when the concentration of MVs positive for a protein in the plasma is above a threshold value, wherein the protein is carcinoembryonic antigen (CEA), and wherein the method has a ROC sensitivity of 25% or more and a specificity of 100%. The individual may be classified as having CRC when the concentration of CEA-positive MVs in the plasma is 221 MVs/μL or more, and wherein the method has a ROC sensitivity of 25% or more and a specificity of 100%. The method may also be characterised as having an AUC of 0.75 or more. The method may also be characterised as having an AUC of 0.75 or more and wherein the 95% confidence intervals are 0.57-0.93. In any of these methods the p-value may be ≤0.02.

In any of the above-described methods, the method may be characterised as having an ROC AUC of 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more, 0.90 or more, 0.91 or more, 0.92 or more, 0.93 or more, 0.94 or more, 0.95 or more, 0.96 or more, 0.97 or more, 0.98 or more, 0.99 or 1.00.

### Control

In any of the methods of the invention that determine the concentration of MVs in the individual's plasma compared to control or the concentration of MVs that test positive for the detectable expression, preferably detectable surface expression, of one or more biomarkers compared to control, a “control” may refer to the comparative concentration of MVs in a healthy individual that does not have BCRPs or CRC. A control may also mean the average comparative concentration of MVs in more than one healthy individual, e.g. an average of a group of healthy individuals. The control may also refer to the comparative concentration of MVs obtained from the same individual as the test individual, but wherein the MVs have been obtained from a sample taken from the same individual at a different time e.g. 1 year, 2 years, 3 years, 4 years or 5 years before the method of the invention is performed.

In a method for classifying the individual as having BCRPs or CRC when the concentration of MVs in the individual's plasma is statistically significantly higher compared to control, the plasma concentration of MVs in the control may be any concentration that is observed for a control as defined above.

The plasma concentration of MVs in the control may be 250 MVs/μL or less, 225 MVs/μL or less, 200 MVs/μL or less, 175 MVs/μL or less, 150 MVs/μL or less, 125 MVs/μL or less, or 124 MVs/μL or less.

### Detection and Quantification of Biomarkers

A sample of MVs can be obtained using any of the methods described herein.

After obtaining a sample of MVs, the concentration of MVs per se, as well as the concentration of MVs which test positive for the detectable expression, preferably detectable surface expression of one or more biomarkers, can be determined. This can be determined in a way that the user deems appropriate. For example, MVs themselves, as well as the one or more biomarkers expressed on the surface of MVs can be stained using binding molecules, such as antibodies, and identified using suitable assays such as immunoassays, e.g. enzyme-linked immunosorbent assay (ELISA) or by cytometry assays e.g. flow cytometry. MVs can also be identified using polymerase chain reaction (PCR)-based methods, for example by detecting and quantifying the amount of messenger RNA and micro-RNA expressed in MVs. MVs and biomarkers are preferably detected via a flow cytometry assay, preferably using antibodies conjugated to a fluorophore/fluorochrome.

For example, a sample of MVs may be labelled with fluorescently-conjugated antibodies. The antibodies may bind to the biomarkers of interest on the surface of the MVs. For example, the antibodies can be those that bind to CEA, A33, LGR5, EPhB2, ICAM-1, CD31, CD42a, CD31+/CD42a−, CK20, cytokeratin 7, CK20+/CK7−, HLA-DR and/or CD147. MVs themselves can be detected using an anti-annexin V antibody, irrespective of the particular biomarkers which may be expressed on the surface of the MVs.

A variety of fluorochromes can be used for conjugation to the antibodies, such as phycoerythrin (PE), allophycyanin (APC), or APC-Cy7, in order to allow for multiple labelling of biomarkers simultaneously. To distinguish non-specific staining, isotype control antibodies may be used. After removing unbound antibodies, samples can be read by a flow cytometer in order to detect MVs stained with antibodies.

Immunoassays such as enzyme-linked immunosorbent assay (ELISA) can be used to detect the presence and/or concentration of target molecules.

In immunoassays, target molecules are detected using molecules which specifically recognise and bind to the target molecule, often antibodies. Such antibodies may be linked to a detectable label that in turn allows quantification of the target molecule. Labels can include enzymes, radioactive isotopes, fluorophores and many others. If using enzymes, then a substrate is added in order to elicit a detectable signal. For example, the signal can be a colour change, in which case the optical density of the sample can be compared to a standard curve in order to quantify the concentration of the target molecule in a sample.

PCR-based methods can also be used to detect the presence and/or concentration of target molecules' related mRNA expression. In such methods, mRNA for Annexin V, CEA, A33, LGR5, EPhB2, ICAM-1, CD31, CD42a, CD31+/CD42a−, CK20, cytokeratin 7, CK20+/CK7−, HLA-DR and/or CD147 may be extracted from MVs, using routine techniques. The mRNA can be used to perform RT-qPCR in order to detect and quantify the expression of biomarker genes.

In the present invention, biomarkers on the surface of MVs can be detected and quantified using immunoassays, using the techniques described above or other versions of immunoassays which are routine in the art. In the present invention, biomarkers on the surface of MVs are preferably detected and quantified using flow cytometry, as described further herein.

### Detectable Expression and Detectable Surface Expression

In any of the methods of the invention disclosed herein, the term “detectable expression” refers to expression of biomarkers within MVs or expression of biomarkers on the cell membrane of MVs. The biomarkers are detectable by routine methods in the art as discussed above, such as PCR-based protocols or antibody-based flow cytometry protocols.

In any of the methods of the invention disclosed herein, the term “detectable surface expression” refers to expression of biomarkers on the cell membrane of MVs, that are detectable by routine methods in the art as discussed above, such as antibody-based flow cytometry protocols.

### Factor Analysis and Component Factors 1 and 2

In order to achieve the most accurate diagnostic value of multiple MVs markers, the inventors performed principle component analysis. See Example 3 for a description of how the analysis was conducted.

The Component Factor 1 (CF1) group of biomarkers comprises or consists of the biomarkers CD31, CD42a, CD31+/CD42a−, EPHB2, ICAM and LGR5. The Component Factor 2 (CF2) group of biomarkers comprises or consists of the biomarkers EPHB2, A33, CEA, and LGR5.

As shown in Example 3, and as explained above, use of the Component Factor 1 group of biomarkers was able to significantly (p<0.0001) diagnose BCRP and CRC with AUC of 100 and 95% respectively. Similarly, use of the Component Factor 2 group of biomarkers was able to significantly (p<0.0001) diagnose BCRP and CRC with AUC of 94% and 93% respectively.

### Detection and Quantification of Blood Components

In any of the methods of the invention, specific blood components can be measured to distinguish whether an individual has BRCP or CRC. The specific blood components that can be measured are blood proteins, including haemoglobin, albumen, C-reactive protein (CRP) and carcinoembryonic antigen (CEA); blood cells including neutrophils and lymphocytes; and blood compounds including urea and creatinine.

To determine the concentration of these components in blood, a sample of blood which has been obtained from an individual is firstly provided. The sample may be processed and purified in any way that the user deems appropriate, such that the concentration of the blood component can be determined and/or determined as being above or below a threshold value. This may involve centrifugation and other routine techniques in order to obtain a purified sample of the appropriate blood component. The detection and/or quantification of the blood components may preferably be carried out in the absence of a cell lysis step, for example a blood cell lysis step.

For example, blood proteins, including haemoglobin, albumen and C-reactive protein (CRP) may be detected and quantified by an immunoassay, preferably an enzyme-linked immunosorbent assay (ELISA); or via a cytometry assay, such as a mass cytometry assay or a flow cytometry assay; or by a mass spectrometry assay; or by spectrophotometry. For example, the following analysers may be used: PerkinElmer AutoDELFIA analysers, DELFIA Xpress analysers, Roche Cobas e411 analyser and API 4000 LC-tandem mass spectrometers.

Spectrophotometry is a method that measures the amount of light which is absorbed by a substance, by measuring the intensity of light that passes through a solution which contains that substance. The concentration of that substance can be determined by comparison to a standard curve of known concentrations.

In the present methods, the concentration of blood components can be measured using spectrophotometry. The absorption of light by a sample of the blood component can be measured, and plotted against a standard curve, in order to calculate the concentration of the blood component in the sample.

In immunoassays, target molecules are detected using molecules which specifically recognise and bind to the target molecule, often antibodies. Such antibodies may be linked to a detectable label that in turn allows quantification of the target molecule. Labels can include enzymes, radioactive isotopes, fluorophores and many others. If using enzymes, then a substrate is added in order to elicit a detectable signal. For example, the signal can be a colour change, in which case the optical density of the sample can be compared to a standard curve in order to quantify the concentration of the target molecule in a sample.

In the present methods, antibodies specific for the blood components can be used to detect their concentration using immunoassays. For example, antibodies specific to the blood component may be fixed to a surface, and a sample of that blood component (e.g. a blood sample) may be washed through, allowing the blood component to bind to the fixed antibody. A secondary antibody, which is linked to a detectable signal, may then be added, which also binds to the blood component (so-called sandwich immunoassay). The detectable signal is then measured in order to calculate the concentration of the blood component in the sample.

Mass cytometry is a variation of flow cytometry in which antibodies are labelled with metal isotopes instead of fluorochromes. Antibodies are then used to label proteins. Signals from the metal are analysed using time-of-flight mass spectrometry. In the present invention, blood proteins may be labelled using metal-isotope-labelled antibodies and detected using mass cytometry.

Mass spectrometry is a technique that measure the mass-to-charge ratio of molecules in a sample. Molecules are ionised, sorted and separated according to mass-to-charge ratio, which is then plotted against their relative abundance. Mass spectrometry can be used to detect and quantify blood components in the present invention.

The blood component carcinoembryonic antigen (CEA) is found expressed on the surface of MVs and is therefore detected and quantified in the same way described herein for the detection and quantification of biomarker-positive MVs.

Blood cells including neutrophils and lymphocytes can also be detected and quantified by an immunoassay, preferably an enzyme-linked immunosorbent assay (ELISA); or via a cytometry assay, such as a mass cytometry assay or a flow cytometry assay; or by a mass spectrometry assay; or by spectrophotometry, as described above.

Blood compounds including urea and creatinine are detected and quantified by a mass spectrometry assay; or by nuclear magnetic resonance; or by spectrophotometry.

Nuclear magnetic resonance (NMR) is a technique that measures the interactions of nuclear spins within the nuclei of atoms within a molecule when placed in a strong magnetic field. In the present invention, NMR can be used to detect and quantify blood compounds of interest.

### Systems

The invention also provides systems and apparatus for performing the methods of the invention, in particular systems for detecting and quantifying biomarker-positive MVs.

For example, the invention provides a chip in order to detect biomarker-positive MVs. The chip may comprise one or more input channels for introduction of MVs, reagents for determining the concentration of MVs, and/or introduction of wash buffer. The chip may also comprise one or more chambers for mixing the MVs with the reagents and/or washing the MVs using the wash buffer. The chip may also comprise one or more output channels for releasing MVs after mixing and washing, for introduction into a flow cytometer and/or for releasing the reagents and wash buffer after mixing and washing.

For example, the chip may comprise a first input channel for introduction of MVs; a second input channel for introduction of reagents for determining the concentration of MVs in an individual's plasma or determining the concentration of MVs in the individual's plasma which test positive for the detectable surface expression of one or more biomarkers;
(c) a third input channel for introduction of wash buffer;
(d) a first chamber for mixing the MVs with the reagents;
(e) a second chamber for washing the MVs from the reagents using the wash buffer;
(f) a first output channel for releasing MVs after mixing and washing, for introduction into a flow cytometer; and
(g) a second output channel for releasing the reagents and wash buffer after mixing and washing.

### Kits of the Invention

Any of the methods disclosed herein can be formulated as a kit. The kit may comprise reagents for carrying out the methods.

The reagents may comprise buffers for reconstituting MVs. The reagents may also comprise antibodies and fluorochromes for identifying the concentration of MVs which test positive for the detectable expression, preferably detectable surface expression, of one or more biomarkers. The reagents may include those required for performing flow cytometry. The reagents may include those required for detection and quantification of blood components, as described and defined further herein.

For example, the invention provides a kit comprising reagents for:
a) detecting microvesicles (MVs) and determining the concentration of MVs in an individual's plasma; and
b) detecting the presence of one, more or all biomarkers in a group of biomarkers expressed in MVs, wherein the group of biomarkers consists of CEA, A33, LGR5, EPhB2, ICAM-1, CD31, CD42a, CD31+/CD42a−, CK20, CK7, CK20+/CK7−, HLA-DR or CD147.

The kit may comprise reagents for detecting the presence of the Component Factor 1 (CF1) group of biomarkers. The kit may further comprise reagents for detecting the presence of one, more or all of the biomarkers CEA, A33, CK20, CK7, CK20+/CK7−, HLA-DR and CD147

The kit may comprise reagents for detecting the presence of the Component Factor 1 (CF2) group of biomarkers. The kit may further comprise reagents for detecting the presence of one, more or all of the biomarkers ICAM-1, CD31, CD42a, CD31+/CD42a−, CK20, CK7, CK20+/CK7−, HLA-DR and CD147.

The kit may further comprise reagents for detecting and quantifying in blood:
a) a blood protein, wherein the protein is haemoglobin, albumen, C-reactive protein (CRP) and/or carcinoembryonic antigen (CEA); and/or
b) a blood cell, wherein the cell is a neutrophil and/or a lymphocyte; and/or
c) a blood compound, wherein the compound is urea and/or creatinine.

The reagent for detecting MVs and determining the concentration of MVs in an individual's plasma in the kit may comprise an anti-Annexin V antibody, optionally conjugated to a fluorophore, preferably fluorescein isothiocyanate (FITC).

The reagents for detecting the presence of the one, more or all biomarkers expressed in MVs in the kit may comprise an anti-CEA antibody, an anti-A33 antibody, an anti-LGR5 antibody, an anti-EPhB2 antibody, an anti-ICAM-1 antibody, an anti-CD31 antibody, an anti-CD42a antibody, an anti-CK20 antibody, an anti-CK7 antibody, an anti-HLA-DR antibody and an anti-CD147 antibody, preferably wherein the antibodies are labelled with a fluorophore.

The reagents for detecting the presence of the one, more or all biomarkers expressed in MVs in the kit may comprise:
a) forward and reverse primers for amplifying, in a nucleic acid amplification reaction, nucleic acid encoding the one or more biomarkers;
b) a detectable probe for detecting amplification products generated by the forward and reverse primers in the nucleic acid amplification reaction; and optionally
c) a nucleic acid amplification enzyme, preferably a DNA polymerase Such a kit may comprise a nucleic acid amplification enzyme, preferably a DNA polymerase, and further comprising a reverse transcriptase enzyme.

### Embodiments of the Invention

1. A method of assessing whether an individual has colorectal cancer (CRC) or is at risk of developing CRC, the method comprising:
   1) providing a sample of microvesicles (MVs) which has been obtained from the plasma of the individual;
   2) determining the concentration of MVs in the individual's plasma; and
   3) classifying the individual as having benign colorectal polyps (BCRPs) or CRC when the concentration of MVs in the individual's plasma is statistically significantly higher compared to control.
2. A method according to embodiment 1, wherein the plasma concentration of MVs in the control is 124 MVs/μL or less.
3. A method according to embodiment 1 or embodiment 2, wherein when the plasma concentration of MVs is 144 MVs/μL or more the individual is identified as having BCRPs or CRC, and wherein the method has a receiver operating characteristics (ROC) sensitivity of 100% and a specificity of 59%.
4. A method according to embodiment 1 or embodiment 2, wherein when the plasma concentration of MVs is 244 MVs/μL or more the individual is identified as having BCRPs or CRC, and wherein the method has a ROC sensitivity of 100% and a specificity of 93%.
5. A method according to any one of embodiments 1 to 5, further comprising:
   1) determining the concentration of MVs in the individual's plasma which test positive for the detectable expression, preferably detectable surface expression, of one or more biomarkers; and
   2) classifying the individual as having BCRPs or CRC when the concentration of biomarker positive MVs in the individual's plasma is statistically significantly higher compared to control and/or when the concentration of biomarker positive MVs in the individual's plasma exceeds a threshold value.
6. A method according to embodiment 5, wherein:
   1) the one or more biomarkers comprises CEA, and wherein the individual is classified as having CRC when the concentration of MVs which test positive for the detectable expression of CEA exceeds a threshold value, and wherein the method has a ROC sensitivity of 100% and a specificity of 60% or more, preferably wherein the threshold value is 10 MVs/μL or more; and/or
   2) the one or more biomarkers comprises A33, and wherein the individual is classified as having CRC when the concentration of MVs which test positive for the detectable expression of A33 exceeds a threshold value, and wherein the method has a ROC sensitivity of 100% and a specificity of 13% or more, preferably wherein the threshold value is 13 MVs/μL or more; and/or
   3) the one or more biomarkers comprises LGR5, and wherein the individual is classified as having CRC when the concentration of MVs which test positive for the detectable expression of LGR5 exceeds a threshold value, and wherein the method has a ROC sensitivity of 96% or more and a specificity of 53% or more, preferably wherein the threshold value is 19 MVs/μL or more; and/or
   4) the one or more biomarkers comprises EPhB2, and wherein the individual is classified as having CRC when the concentration of MVs which test positive for the detectable expression of EPhB2 exceeds a threshold value, and wherein the method has a ROC sensitivity of 100% and a specificity of 13% or more, preferably wherein the threshold value is 52 MVs/μL or more; and/or
   5) the one or more biomarkers comprises ICAM-1, and wherein the individual is classified as having CRC when the concentration of MVs which test positive for the detectable expression of ICAM-1 exceeds a threshold value, and wherein the method has a ROC sensitivity of 96% or more and a specificity of 47% or more, preferably wherein the threshold value is 12 MVs/μL or more; and/or
   6) the one or more biomarkers comprises CD31, and wherein the individual is classified as having CRC when the concentration of MVs which test positive for the detectable expression of CD31 exceeds a threshold value, and wherein the method has a ROC sensitivity of 100% and a specificity of 60% or more, preferably wherein the threshold value is 38 MVs/μL or more; and/or
   7) the one or more biomarkers comprises CD42a, and wherein the individual is classified as having CRC when the concentration of MVs which test positive for the detectable expression of CD42a exceeds a threshold value, and wherein the method has a ROC sensitivity of 100% and a specificity of 20% or more, preferably wherein the threshold value is 9 MVs/μL or more; and/or
   8) the one or more biomarkers comprises CD31+/CD42a−, and wherein the individual is classified as having CRC when the concentration of MVs which test positive for the detectable expression of CD31+/CD42a− exceeds a threshold value, and wherein the method has a ROC sensitivity of 96% or more and a specificity of 87% or more, preferably wherein the threshold value is 38 MVs/μL or more; and/or
   9) the one or more biomarkers comprises CK20, and wherein the individual is classified as having CRC when the concentration of MVs which test positive for the detectable expression of CK20 exceeds a threshold value, and wherein the method has a ROC sensitivity of 100% and a specificity of 47% or more, preferably wherein the threshold value is 8 MVs/μL or more; and/or
   10) the one or more biomarkers comprises CK7, and wherein the individual is classified as having CRC when the concentration of MVs which test positive for the detectable expression of cytokeratin 7 exceeds a threshold value, and wherein the method has a ROC sensitivity of 100% and a specificity 47% or more, preferably wherein the threshold value is 16 MVs/μL or more; and/or
   11) the one or more biomarkers comprises CK20+/CK7−, and wherein the individual is classified as having CRC when the concentration of MVs which test positive for the detectable expression of Ck20+/CD7− exceeds a threshold value, and wherein the method has a ROC sensitivity of 100% and a specificity of 33% or more, preferably wherein the threshold value is 4 MVs/μL or more; and/or
   12) the one or more biomarkers comprises HLA-DR, and wherein the individual is classified as having CRC when the concentration of MVs which test positive for the detectable expression of HLA-DR exceeds a threshold value, and wherein the method has a ROC sensitivity of 100% and a specificity of 27% or more, preferably wherein the threshold value is 26 MVs/μL or more; and/or
   13) the one or more biomarkers comprises CD147, and wherein the individual is classified as having CRC when the concentration of MVs which test positive for the detectable expression of CD147 exceeds a threshold value, and wherein the method has a ROC sensitivity of 96% or more and a specificity of 27% or more, preferably wherein the threshold value is 17 MVs/μL or more.
7. A method according to embodiment 5, wherein:
   1) the one or more biomarkers comprises CEA, and wherein the individual is classified as having BCRPs when the concentration of MVs which test positive for the detectable expression of CEA exceeds a threshold value, and wherein the method has a ROC sensitivity of 100% and a specificity of 40% or more, preferably wherein the threshold value is 6.37 MVs/μL or more; and/or
   2) the one or more biomarkers comprises LGR5, and wherein the individual is classified as having BCRPs when the concentration of MVs which test positive for the detectable expression of LGR5 exceeds a threshold value, and wherein the method has a ROC sensitivity of 100% and a specificity of 60% or more, preferably wherein the threshold value is 28.4 MVs/μL or more; and/or
   3) the one or more biomarkers comprises EPhB2, and wherein the individual is classified as having BCRPs when the concentration of MVs which test positive for the detectable expression of EPhB2 exceeds a threshold value, and wherein the method has a ROC sensitivity of 100% and a specificity of 47% or more, preferably wherein the threshold value is 1.07 MVs/μL or more; and/or
   4) the one or more biomarkers comprises ICAM-1, and wherein the individual is classified as having BCRPs when the concentration of MVs which test positive for the detectable expression of ICAM-1 exceeds a threshold value, and wherein the method has a ROC sensitivity of 100% and a specificity of 13% or more, preferably wherein the threshold value is 3.6 MVs/μL or more; and/or
   5) the one or more biomarkers comprises CD31, and wherein the individual is classified as having BCRPs when the concentration of MVs which test positive for the detectable expression of CD31 exceeds a threshold value, and wherein the method has a ROC sensitivity of 100% and a specificity of 87% or more, preferably wherein the threshold value is 72 MVs/μL or more; and/or
   6) the one or more biomarkers comprises CD42a, and wherein the individual is classified as having BCRPs when the concentration of MVs which test positive for the detectable expression of CD42a exceeds a threshold value, and wherein the method has a ROC sensitivity of 100% and a specificity of 47% or more, preferably wherein the threshold value is 25 MVs/μL or more; and/or
   7) the one or more biomarkers comprises CD31+/CD42a−, and wherein the individual is classified as having BCRPs when the concentration of MVs which test positive for the detectable expression of CD31+/CD42a− exceeds a threshold value, and wherein the method has a ROC sensitivity of 100% and a specificity of 87% or more, preferably wherein the threshold value is 44 MVs/μL or more; and/or
   8) the one or more biomarkers comprises CK7, and wherein the individual is classified as having BCRPs when the concentration of MVs which test positive for the detectable expression of CK7 exceeds a threshold value, and wherein the method has a ROC sensitivity of 100% and a specificity of 33% or more, preferably wherein the threshold value is 11 MVs/μL or more; and/or
   9) the one or more biomarkers comprises HLA-DR, and wherein the individual is classified as having BCRPs when the concentration of MVs which test positive for the detectable expression of HLA-DR exceeds a threshold value, and wherein the method has a ROC sensitivity of 100% and a specificity of 7% or more, preferably wherein the threshold value is 17 MVs/μL or more; and/or
   10) the one or more biomarkers comprises CD147, and wherein the individual is classified as having BCRPs when the concentration of MVs which test positive for the detectable expression of CD147 exceeds a threshold value, and wherein the method has a ROC sensitivity of 100% and a specificity of 13% or more, preferably wherein the threshold value is 7.4 MVs/μL or more.
8. A method according to any one of embodiments 5, 6 or 7, wherein the individual is classified as having BCRPs when the concentration of MVs which test positive for the detectable expression of all biomarkers comprising the Component Factor 2 (CF2) group of biomarkers exceeds a threshold value and wherein the method has a ROC sensitivity of 100% and a specificity of 63% or more, preferably wherein the threshold value is 222 MVs/μL or more.
9. A method according to any one of embodiments 5, 6 or 7, wherein the individual is classified as having BCRPs when the concentration of MVs which test positive for the detectable expression of all biomarkers comprising the Component Factor 1 (CF1) group of biomarkers exceeds a threshold value and wherein the method has a ROC sensitivity of 100% and a specificity of 100%, preferably wherein the threshold value is 761 MVs/μL or more.
10. A method according to any one of embodiments 5, 6 or 7, wherein the individual is classified as having CRC when the concentration of MVs which test positive for the detectable expression of all biomarkers comprising the Component Factor 2 (CF2) group of biomarkers exceeds a threshold value and wherein the method has a ROC sensitivity of 100% and a specificity of 50% or more, preferably wherein the threshold value is 157 MVs/μL or more.
11. A method according to any one of embodiments 5, 6 or 7, wherein the individual is classified as having CRC when the concentration of MVs which test positive for the detectable expression of all biomarkers comprising the Component Factor 1 (CF1) group of biomarkers exceeds a threshold value and wherein the method has a ROC sensitivity of 100% and a specificity of 56% or more, preferably wherein the threshold value is 439 MVs/μL or more.
12. A method according to any one of embodiments 1 to 11, the method further comprising providing a sample of blood which has been obtained from the individual, determining the concentration of a protein in the individual's blood and classifying the individual as having CRC when the concentration of the protein is below a threshold value, wherein the protein is haemoglobin and wherein the method has a ROC sensitivity of 68% or more and a specificity of 100%.
13. A method according to embodiment 12, wherein the individual is distinguished as having CRC rather than BCRPs when the concentration of haemoglobin is 133 g/L or less, and wherein the method has a ROC sensitivity of 58.3% or more and a specificity of 90% or more; or wherein the individual is classified as having CRC when the concentration of haemoglobin is 124.5 g/L or less, and wherein the method has a ROC sensitivity of 68% or more and a specificity of 100%.
14. A method according to any one of embodiments 1 to 13, the method further comprising providing a sample of blood which has been obtained from the individual, determining the concentration of a blood cell type in the individual's blood and classifying the individual as having CRC when the concentration of the blood cell type is above a threshold value, wherein the blood cell type is neutrophils and wherein the method has a ROC sensitivity of 47% or more and a specificity of 100%,
15. A method according to embodiment 14, wherein the individual is classified as having CRC when the concentration of neutrophils is 7.7×10⁹/L or more, and wherein the method has a ROC sensitivity of 47% or more and a specificity of 100%.
16. A method according to any one of embodiments 1 to 15, the method further comprising providing a sample of blood which has been obtained from the individual, determining the concentration of a blood cell type in the individual's blood and classifying the individual as having CRC when the concentration of the blood cell type is below a threshold value, wherein the blood cell type is lymphocytes and wherein the method has a ROC sensitivity of 47% or more and a specificity of 100%.
17. A method according to embodiment 16, wherein the individual is classified as having CRC when the concentration of lymphocytes is 1.16×10⁹/L or less, and wherein the method has a ROC sensitivity of 47% or more and a specificity of 100%.
18. A method according to any one of embodiments 1 to 17, the method further comprising providing a sample of blood which has been obtained from the individual, determining the concentration of a protein in the individual's blood and classifying the individual as having CRC when the concentration of the protein is below a threshold value, wherein the protein is albumen and wherein the method has a ROC sensitivity of 63% or more and a specificity of 100%.
19. A method according to embodiment 18, wherein the individual is distinguished as having CRC rather than BCRPs when the concentration of albumen is 43.5 g/L or less, and wherein the method has a ROC sensitivity of 76.9% or more and a specificity of 90% or more; or wherein the individual is classified as having CRC when the concentration of albumen is 39.5 g/L or less, and wherein the method has a ROC sensitivity of 63% or more and a specificity of 100%,
20. A method according to any one of embodiments 1 to 19, the method further comprising providing a sample of blood which has been obtained from the individual, determining the concentration of a protein in the individual's blood and classifying the individual as having CRC when the concentration of the protein is above a threshold value, wherein the protein is C-reactive protein (CRP) and wherein the method has a ROC sensitivity of 63% or more and a specificity of 100%,
21. A method according to embodiment 20, wherein the individual is distinguished as having CRC rather than BCRPs when the concentration of CRP is 19.65 mg/L or more, and wherein the method has a ROC sensitivity of 85.7% or more and a specificity of 90% or more; or wherein the individual is classified as having CRC when the concentration of CRP is 72.75 mg/L or more, and wherein the method has a ROC sensitivity of 63% or more and a specificity of 100%.
22. A method according to any one of embodiments 1 to 21, the method further comprising providing a sample of blood which has been obtained from the individual, determining the concentration of a compound in the individual's blood and classifying the individual as having CRC when the concentration of the compound is below a threshold value, wherein the compound is urea and wherein the method has a ROC sensitivity of 58% or more and a specificity of 100%.
23. A method according to embodiment 22, wherein the individual is distinguished as having CRC rather than BCRPs when the concentration of urea is 5.5 mmol/L or less, and wherein the method has a ROC sensitivity of 54.55% or more and a specificity of 90% or more; or wherein the individual is classified as having CRC when the concentration of urea is 3.85 mmol/L or less, and wherein the method has a ROC sensitivity of 58% or more and a specificity of 100%.
24. A method according to any one of embodiments 1 to 23, the method further comprising providing a sample of blood which has been obtained from the individual, determining the concentration of a compound in the individual's blood and classifying the individual as having CRC when the concentration of the compound is below a threshold value, wherein the compound is creatinine and wherein the method has a ROC sensitivity of 53% or more and a specificity of 100%,
25. A method according to embodiment 24, wherein the individual is distinguished as having CRC rather than BCRPs when the concentration of creatinine is 82.5 mmol/L or less, and wherein the method has a ROC sensitivity of 75% or more and a specificity of 90.0% or more; or wherein the individual is classified as having CRC when the concentration of creatinine is 63.5 μmol/L or less, and wherein the method has a ROC sensitivity of 53% or more and a specificity of 100%.
26. A method according to any one of embodiments 1 to 25, the method further comprising providing a sample of blood which has been obtained from the individual, determining the concentration of MVs in the plasma of the individual which test positive for the detectable expression of a protein and classifying the individual as having CRC when the concentration of MVs positive for the protein in the plasma is above a threshold value, wherein the protein is carcinoembryonic antigen (CEA) and wherein the method has a ROC sensitivity of 25% or more and a specificity of 100%.
27. A method according to embodiment 26, wherein the individual is classified as having CRC when the concentration of CEA-positive MVs in the plasma is 221 MVs/μL or more, and wherein the method has a ROC sensitivity of 25% or more and a specificity of 100%.
28. A method according to any one of embodiments 5 to 11, wherein the expression of the one or more biomarkers is detected via PCR, preferably RT-qPCR; or via an immunoassay, preferably an enzyme-linked immunosorbent assay (ELISA); or via a cytometry assay, such as a mass cytometry assay or a flow cytometry assay; or by a mass spectrometry assay; preferably the one or more biomarkers are detected via a flow cytometry assay, preferably using an anti-biomarker antibody conjugated to a fluorophore.
29. A method according to any one of embodiments 12 to 21, 26 and 27, wherein the protein and/or the blood cell type is detected via an immunoassay, preferably an enzyme-linked immunosorbent assay (ELISA); or via a cytometry assay, such as a mass cytometry assay or a flow cytometry assay; or by a mass spectrometry assay; or by spectrophotometry.
30. A method according to any one of embodiments 22 to 25, wherein the compound is detected via a mass spectrometry assay; or by nuclear magnetic resonance; or by spectrophotometry.
31. A method according to any one of the preceding embodiments, wherein MVs are detected via an immunoassay, preferably an enzyme-linked immunosorbent assay (ELISA); or via a cytometry assay, such as a mass cytometry assay or a flow cytometry assay; or by a mass spectrometry assay; preferably MVs are detected via a flow cytometry assay, preferably using an anti-annexin V antibody conjugated to a fluorophore such as fluorescein isothiocyanate (FITC).
32. A method according to any one of the preceding embodiments, wherein the individual is a mammal, such as a feline mammal, a canine mammal, a porcine mammal, an equine mammal, a bovine mammal, a murine mammal e.g. a mouse or a rat, or a primate mammal e.g. a monkey or chimpanzee, preferably wherein the individual is a human.
33. A kit comprising reagents for:
   a) detecting microvesicles (MVs) and determining the concentration of MVs in an individual's plasma; and
   b) detecting the presence of one, more or all biomarkers in a group of biomarkers expressed in MVs, wherein the group of biomarkers consists of CEA, A33, LGR5, EPhB2, ICAM-1, CD31, CD42a, CD31+/CD42a−, CK20, CK7, CK20+/CK7−, HLA-DR or CD147.
34. A kit according to embodiment 33, comprising reagents for detecting the presence of the Component Factor 1 (CF1) group of biomarkers.
35. A kit according to embodiment 34, further comprising reagents for detecting the presence of one, more or all of the biomarkers CEA, A33, CK20, CK7, CK20+/CK7−, HLA-DR and CD147
36. A kit according to embodiment 33, comprising reagents for detecting the presence of the Component Factor 1 (CF2) group of biomarkers.
37. A kit according to embodiment 36, further comprising reagents for detecting the presence of one, more or all of the biomarkers ICAM-1, CD31, CD42a, CD31+/CD42a−, CK20, CK7, CK20+/CK7−, HLA-DR and CD147.
38. A kit according to any one of embodiments 33 to 37, further comprising reagents for detecting and quantifying in blood:
   a) a blood protein, wherein the protein is haemoglobin, albumen, C-reactive protein (CRP) and/or carcinoembryonic antigen (CEA); and/or
   b) a blood cell, wherein the cell is a neutrophil and/or a lymphocyte; and/or
   c) a blood compound, wherein the compound is urea and/or creatinine.
39. A kit according to any one of embodiments 33 to 38, wherein the reagent for detecting MVs and determining the concentration of MVs in an individual's plasma comprises an anti-Annexin V antibody, optionally conjugated to a fluorophore, preferably fluorescein isothiocyanate (FITC).
40. A kit according to any one of embodiments 33 to 39, wherein the reagents for detecting the presence of the one, more or all biomarkers expressed in MVs comprise respectively an anti-CEA antibody, an anti-A33 antibody, an anti-LGR5 antibody, an anti-EPhB2 antibody, an anti-ICAM-1 antibody, an anti-CD31 antibody, an anti-CD42a antibody, CD31+/CD42a−, an anti-CK20 antibody, an anti-CK7 antibody, CK20+/CK7−, an anti-HLA-DR antibody and an anti-CD147 antibody, preferably wherein the antibodies are labelled with a fluorophore.
41. A kit according to any one of embodiments 33 to 39, wherein the reagents for detecting the presence of the one, more or all biomarkers expressed in MVs comprise:
   a) forward and reverse primers for amplifying, in a nucleic acid amplification reaction, nucleic acid encoding the one or more biomarkers;
   b) a detectable probe for detecting amplification products generated by the forward and reverse primers in the nucleic acid amplification reaction; and optionally
   c) a nucleic acid amplification enzyme, preferably a DNA polymerase
42. A kit according to embodiment 41, comprising a nucleic acid amplification enzyme, preferably a DNA polymerase, and further comprising a reverse transcriptase enzyme.
43. A chip comprising:
   a) a first input channel for introduction of MVs;
   b) a second input channel for introduction of reagents for determining the concentration of MVs in an individual's plasma, or determining the concentration of MVs in the individual's plasma which test positive for the detectable surface expression of one or more biomarkers;
   c) a third input channel for introduction of wash buffer;
   d) a first chamber for mixing the MVs with the reagents;
   e) a second chamber for washing the MVs from the reagents using the wash buffer;
   f) a first output channel for releasing MVs after mixing and washing, for introduction into a flow cytometer; and
   g) a second output channel for releasing the reagents and wash buffer after mixing and washing.

### EXAMPLES

### Example 1. Materials and Methods

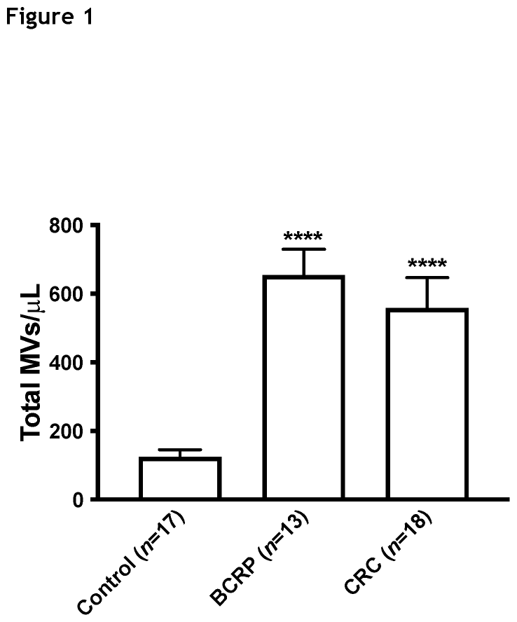

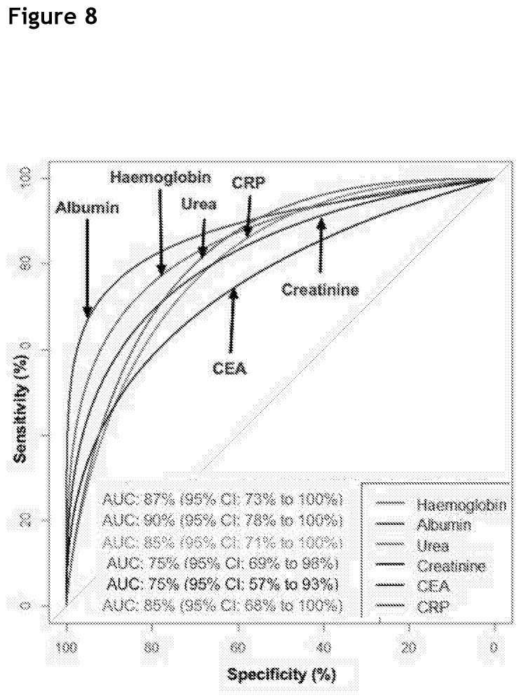

The following describes the materials and methods used for Examples 2 to 4, and FIGS. 1 to 9

### Study Participants and Patient Sample

A total number of 35 patients with benign CR polyps (n=16) and CRC (n=19) from University College Hospital, and age-matched 17 healthy control participants from University College London were recruited with the appropriate ethical approval and written consent. Patients with BCRP and CRC had their diagnosis confirmed by histology. Healthy controls were included if they are age matched and have no medical history of bowel disease. Patients and participants with cardiovascular and/or inflammatory conditions were excluded.

Blood samples were collected from patients in a lavender EDTA vacutainer tubes (BD, Oxford, UK), and platelet poor plasma (PPP) was obtained by double centrifugation at 5,000×5 minutes using an adopted protocol. MVs were recovered from supernatants or from PPP after centrifugation at 17,000 g×g for 1 hour. MVs were identified using flow cytometry (Brogan P A, Dillon M J. Endothelial microparticles and the diagnosis of the vasculitides. Intern Med 2004; 43(12):1115-9). Anti-annexin V antibody conjugated to fluorescein isothiocyanate (FITC) and diluted in annexin V buffer (BD Pharmingen) was used identify total MVs. 1.1 m latex beads were used to set the upper threshold on forward scatter to distinguish maximum MVs size. MVs captured in this way were defined as annexin V+ particles co-expressing specific cell surface markers, determined by using appropriate isotype control antibodies for each marker. MVs were enumerated in a standardised fashion by using the proportion of a fixed number of 3 m latex beads counted and the quantity of the supernatant or plasma from which the MVs were analysed.

### Flow Cytometric Analysis of Cell Surface Receptors

Plasma was aliquoted into 100 μl aliquots and labelled with fluorescently conjugated antibodies used at 1:50 dilution. Cells and microvesicles were stained with antibodies conjugated with different fluorochromes including phycoerythrin (PE), allophycyanin (APC), or APC-Cy7. To allow for multiple labelling of receptors simultaneously, microvesicles suspended in annexin-V were further labelled with fluorescently conjugated antibodies (1:50 dilution) using different fluorochromes including phycoerythrin (PE), allophycyanin (APC), or APC-Cy7. Mouse antibodies used for staining microvesicles subpopulations were anti-glycoprotein (A33; R&D Systems Abingdon, UK), anti-carcinoembryonic antigen-5 (CEA-5; BD Biosciences, New Jersey, USA), anti-leucine-rich G-protein coupled receptor 5 (LGR-5; BD Biosciences), anti-intercellular adhesion molecule (ICAM1, CD54; BD Biosciences), and anti-platelet endothelial cell adhesion molecule (PECAM1, CD31; BD Biosciences). To distinguish non-specific staining, isotype control antibodies anti-mouse IgG1,k PE (BD Pharmingen), anti-mouse IgG1 APC (R&D Systems Abingdon, UK) and anti-mouse IgG1,k APC-Cy7 (BD Pharmingen, New Jersey, USA) were used with protein: fluorochrome ratios equal to their associated fluorescence conjugated antibodies. The microvesicle-annexin-V-antibody suspensions in 96-well plates were incubated in the dark at room temperature for 15 minutes, after which 200 μl of annexin V buffer was added to each well to neutralise the reaction. The plates were then read by a FACSArray BioAnalyzer™ flow cytometer (BD Biosciences, Oxford, UK). The gating was set by running unstained and isotype control-stained cells through the cytometer and toggling the forward and side scatter and colour channels on logarithmic scales.

### Antibodies and Reagents

Mouse polyclonal phycoerythrin (APC)-labelled anti-glycoprotein A33 and APC-labelled IgG1 isotype control antibodies were obtained from R&D Systems (Abingdon, UK). Mouse monoclonal PE-labelled anti-CEA-5, LGR-5, ICAM-1 (CD54), glycoprotein IX (CD42a) and IgG1κ isotype control antibodies, FITC-annexin V and 10× annexin binding buffer were obtained from BD Pharmingen (New Jersey, USA).

### Statistics

All statistical analysis was carried out using Statistical Package for the Social Sciences” (IBM SPSS Statistics for Macintosh, version25, Armonk, NY: IBM Corp.) and GraphPad Prism (GraphPad Prism version 6 for MAC OSX, GraphPad Software, SanDiego, CA, www.graphpad.com). Data were expressed as mean if parametric and median if non-parametric. Differences in means were assessed using the two way t-test and Mann Whitney U test. Two way ANOVA test was used to examine the difference in mean of groups more than two. Logistic regression, factor analysis and correlation matrix were also performed to assess associations between variables. Sensitivity, specificity, positive and negative predictive values were calculated as appropriate (see Example 2 below). Area under the receiver operator curve (AUC) was also calculated when appropriate using the above mentioned statistical packages. Statistical significance was regarded when P-values were less than 0.05 (two sided).

### Patient Characteristics

Table 1 shows the basic characteristics of the patients recruited to the study. Patients were grouped into four categories: 1) healthy control; 2) patients with BCRP including, neoplastic and non-neoplastic polyps; and 3) patients with confirmed diagnosis of CRC including, MD and PD CRC. Patients had similar age and gender distribution, however, there were significant differences in regard to biochemical markers between patients with BCRP and CRC. As shown in Table 1, the blood level of haemoglobin, lymphocytes, albumin, urea and Creatinine were significantly lower in CRC patients in comparison to BCRP. Other markers including neutrophils and CRP were significantly higher in CRC patients in comparison to BCRP.

TABLE 1
Patients' characteristics

| | Healthy control (n = 17) | BCRP (n = 16) | CRC (n = 19) | p value |
|---|---|---|---|---|
| Age in years: mean (SD) | 56 (10) | 62 (15) | 61 (15) | 0.43 |
| | 9 (53%) | 12 (75%) | 13 (68%) | 0.34 |
| | 8 (47%) | 4 (25%) | 6 (32%) | |
| Histology | — | NNP (n = 5, 38%) | MD CRC (n = 8, 44%) | — |
| | | NP (n = 8, 62%) | PD CRC (n = 10, 56%) | |
| Condition/Cancer stage | — | — | Stage 1 (n = 1, 5%) | — |
| | | | Stage 2 (n = 3, 15%) | |
| | | | Stage 3 (n = 15, 80%) | |
| Haemoglobin [g/L]:** mean (SD) | — | 138 (10) | 117 (22) | 0.006 |
| WCC [10₉/L]: mean (SD) | — | 9 (3) | 9 (3) | 0.55 |
| Neutrophils [10₉/L]:* mean (SD) | — | 5 (2) | 7 (3) | 0.02 |
| Lymphocytes [10₉/L]:** mean (SD) | — | 2 (1) | 1 (0.5) | 0.003 |
| Platelet [10₉/L]: mean (SD) | — | 273 (55) | 320 (168) | 0.38 |
| Albumin [10₉/L]:*** mean (SD) | — | 45 (3) | 37 (6) | 0.0002 |
| CRP [mg/L]:** median (range) | — | 3 (0-67) | 56 (0-295) | 0.009 |
| Creatinine [μmol/L]:** mean (SD) | — | 90 (20) | 75 (20) | 0.003 |
| Urea [mmol/L]:*** mean (SD) | — | 6 (2) | 4 (1) | 0.0003 |

BCRP: Benign colorectal polyps. CRC: colorectal carcinoma. CRP: C-reactive protein. MD: moderately differentiated. NNP: non-neoplastic Polyps. NP: neoplastic polyps. PD: poorly differentiated. SD: standard deviation. WCC: white cell count.
**p < 0.01.
***p < 0.001.

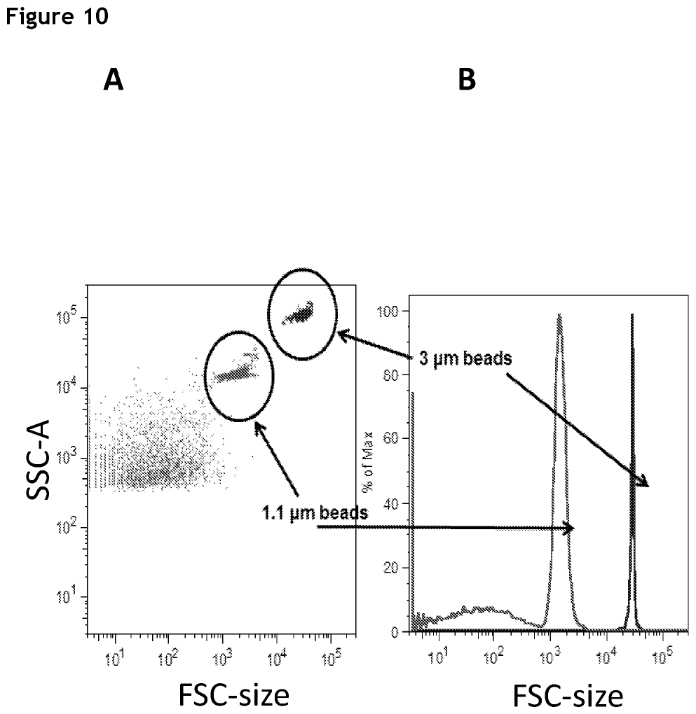

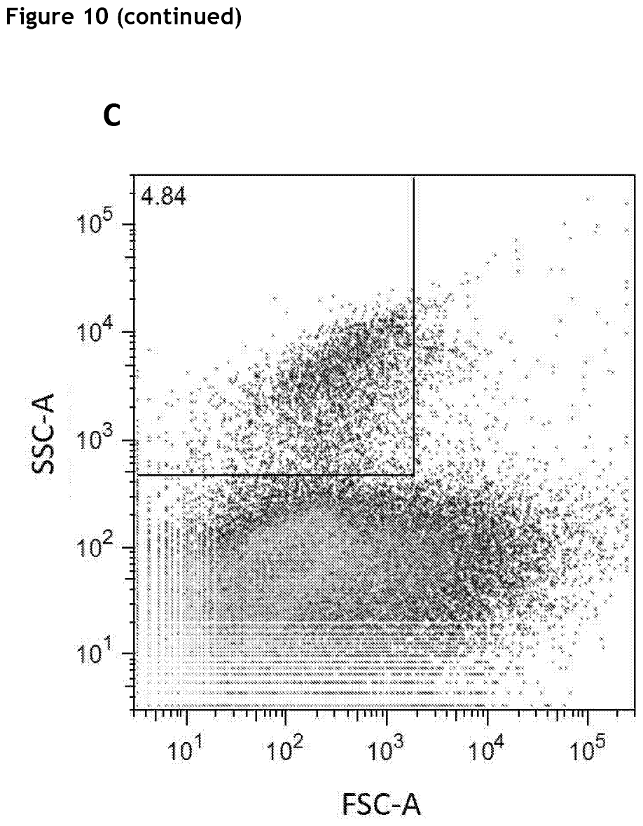

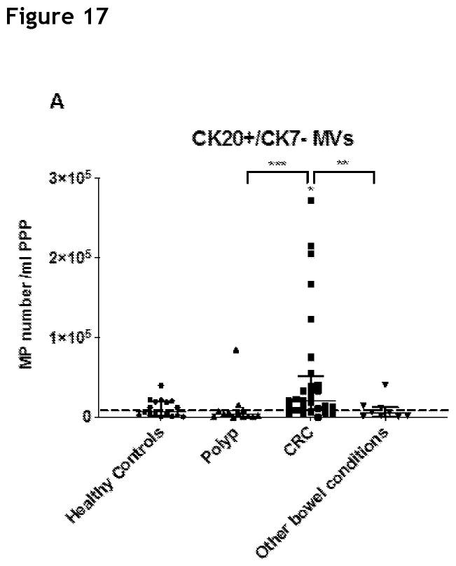

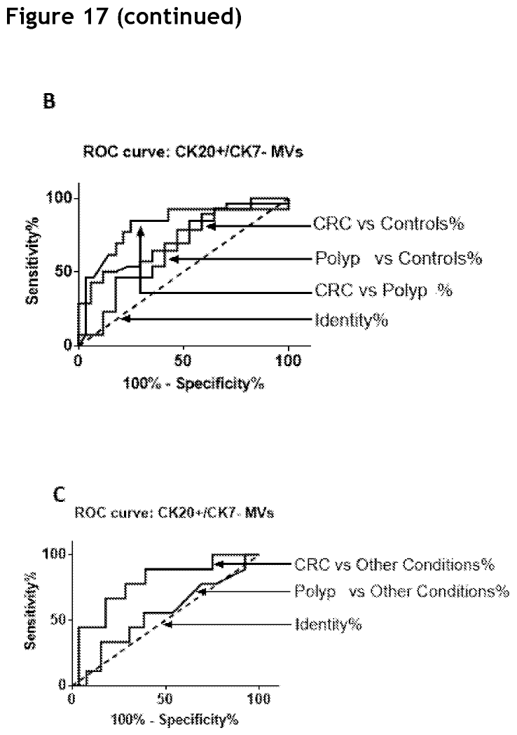

The following describes the materials and methods used for Examples 5 to 20, and FIGS. 10 to 27.

### Study Participants and Patient Sample

A total of 56 patients with colorectal cancer (n=31), polyps (n=16), inflammatory bowel disease and diverticular disease (n=7) from University College Hospital and the Royal Free Hospital and age-matched healthy controls (n=24) from University College London and University College Hospital were recruited with appropriate ethical approval and patient consent from the University College Hospital and Royal Free London National Health Service trust (ethical approval code G12-42333A). The inclusion criteria for blood sampling included: diagnosis confirmed by colonoscopy, CT-scan and/or histology, no prior treatment with chemotherapy and radiotherapy, no major surgery within 2 years prior to recruitment, and no other major cardiovascular and/or inflammatory comorbidities.

Blood samples were collected from patients in a lavender EDTA vacutainer tubes (BD, Oxford, UK), and platelet poor plasma was obtained by double centrifugation at 5,000×5 minutes using an adopted protocol (Brogan P A, Dillon M J. Endothelial microparticles and the diagnosis of the vasculitides. Intern Med 2004; 43(12):1115-9) MVs recovered from platelet poor plasma (PPP) after centrifugation at 17,000 g×g for 1 hour were identified using flow cytometry under the Minimal Information for Studies of Extracellular Vesicles 2018 (MISEV2018) guidelines. Anti-annexin V antibody conjugated to fluorescein isothiocyanate (FITC) and diluted in annexin V buffer (BD Pharmingen) was used identify total MVs. 1.1 μm latex beads were used to set the upper threshold on forward scatter to distinguish maximum MV size (FIG. 10). MVs captured in this way were defined as annexin V+ particles co-expressing specific cell surface markers, determined by using appropriate isotype control antibodies for each marker. MVs were enumerated in a standardised fashion by using the proportion of a fixed number of 3 m latex beads counted and the quantity of the supernatant or plasma from which the MVs were analysed.

### Flow Cytometric Analysis of Cell Surface Receptors

Plasma was aliquoted into 100 μl aliquots and labelled with fluorescently conjugated antibodies used at 1:50 dilution in phosphate buffered saline (PBS). Cells and microvesicles were stained with antibodies conjugated with different fluorochromes including phycoerythrin (PE), allophycyanin (APC), or APC-Cy7 to allow for multiple labelling of receptors simultaneously. Antibodies used for staining MV subpopulations were A33 (R&D Systems Abingdon, UK), anti-CEA-5 (BD Biosciences, New Jersey, USA), LGR-5 (BD Biosciences, New Jersey, USA), anti-EPhB2 (BD Biosciences, New Jersey, USA), cytokeratin 20 (ABCAM, Cambridge, UK), cytokeratin 7 (abcam, Cambridge, UK), CD147 (BD Biosciences, New Jersey, USA), HLA-DR (BD Biosciences, New Jersey, USA), PECAM1 (CD31; BD Biosciences, New Jersey, USA), and CD42a (BD Biosciences, New Jersey, USA). To distinguish non-specific staining, isotype control antibodies anti-mouse IgG1,k PE (BD Pharmingen), anti-mouse IgG1 APC (R&D Systems Abingdon, UK) and anti-mouse IgG1,k APC-Cy7 (BD Pharmingen, New Jersey, USA) were used with equal protein:fluorochrome ratios to their associated fluorescence conjugated antibodies. Samples were transferred to a 96-well plate and read by a FACSArray BioAnalyzer™ flow cytometer. The gating was set by running unstained and isotype control stained cells through the cytometer and toggling the forward & side scatter and colour channels on logarithmic scales.

### Antibodies and Reagents

Mouse polyclonal phycoerythrin (APC)-labelled anti-glycoprotein A33 and APC-labelled IgG1 isotype control antibodies were obtained from R&D Systems (Abingdon, UK). Mouse monoclonal PE-labelled anti-carcinomic embryonic antigen-5 (CEA-5), leucine-rich repeat G-protein coupled receptor 5 (LGR-5), intercellular adhesion molecule-1 (ICAM-1; CD54), glycoprotein IX (CD42a) and IgG1κ isotype control antibodies, FITC-annexin V and 10× annexin binding buffer were obtained from BD Pharmingen (New Jersey, USA).***

### Statistical Analysis

All statistical analysis was carried out on GraphPad Prism 6 software (Jandel software, La Jolla, USA). The MV data were expressed as median±interquartile range. Differences in MV levels between controls, CRC, inflammatory bowel disease (IBD) diverticular disease groups were assessed using the Kruskal-Wallist U test. ROC analyses were performed and ROC curves were obtained for the different disease groups and healthy control groups to obtain sensitivity % and threshold at >90% specificity. Differences in serum markers were assessed using the Mann-Whitney U test. Statistical significance was regarded when P-values were less than 0.05 (two sided).

### Example 2: Plasma Level of Total Microvesicles in Benign Colorectal Polyps and Colorectal Cancer

The plasma levels of total MVs were significantly (p<0.0001) higher in patients BCRP (mean=654 MVs/μL, SD=271) and CRC (mean=558 MVs/μL, SD=378), in comparison to healthy control (mean=124 MVs/μL, SD=88) (FIG. 1). The levels of total plasma MVs were similar in the BCRP and CRP patients.

### Total Plasma Microvesicles for the Diagnosis of Benign Colorectal Polyps and Colorectal Cancer

Elevated total plasma MVs was able to significantly (p<0.0001) diagnose BCRP and CRC with area under the ROC of 99% (95% CI: 96% to 100%) and 94% (95% CI: 86% to 100%) respectively (FIG. 2).

Cut-off point of 211 total plasma MVs/μL demonstrated a sensitivity of 100% (95% CI: 75% to 100%) and a specificity of 88% (95% CI: 64% to 99%) for the diagnosis of BCRP. See Table 2 and equations 1, 2, 3 and 4 below for method of calculation. The cut-off point also shows a likelihood ratio of 8.5. Furthermore, positive and negative predictive values for the diagnosis of BCRP were 87% and 100% respectively (Table 2 and equations 5, 6, 7, and 8). Similarly, a cut-off point of 173 total plasma MVs/μL demonstrated a sensitivity of 89% (95% CI: 65% to 99%), a specificity of 88% (95% CI: 64% to 99%) for the diagnosis of CRC. The likelihood ratio for this cut-off point was 7.5. Positive and negative predictive values were 89% and 88% respectively.

A cut-off point of 144 total plasma MVs/μL provides a 100% sensitivity for BCRP (95% CI: 75% to 100%) and or CRC (95% CI: 81% to 100%). However, the specificity for a cut-off point of 144 total plasma MVs/μL for BCRP and CRC is 59% (95% CI: 33% to 82%: LR: 2.4) and 59% (95% CI: 33% to 82%: LR: 2.4) respectively. PPV and NPV for BCRP are 67% and 100% respectively. For CRC diagnostic accuracy PPV and NPV are 73% and 100% respectively.

TABLE 2
The numbers of participants who's total plasma
microvesicles level were above and below 211 MVs/μL

| | Benign CR polyps | No polyps | Total |
|---|---|---|---|
| Positive (>211 MVs/μL) | 13 | 2 | 15 |
| Negative (>211 MVs/μL) | 0 | 15 | 15 |
| Total | 13 | 17 | 20 |

$$Equation 1. Sensitivity = \frac{True positive}{True positive + false negative} \times 100 \\ Equation 2. Sensitivity = \frac{13}{13 + 0} \times 100 = 100 \% \\ Equation 3. Specificity = \frac{True negative}{True negative + false positive} \times 100 \\ Equation 4. Specificity = \frac{15}{15 + 2} \times 100 = 88 \% \\ Equation 5. Positive predictive value = \frac{True positive}{True positive + false positive} \times 100 \\ Equation 6. Positive predictive value = \frac{13}{13 + 2} \times 100 = 87 \% \\ Equation 7. Negative predictive value = \frac{True negative}{True negative + false negative} \times 100 \\ Equation 8. Negative predictive value = \frac{15}{15 + 0} \times 100 = 100 \%$$

### Example 3: Plasma Level of Microvesicles Positive for Other Markers

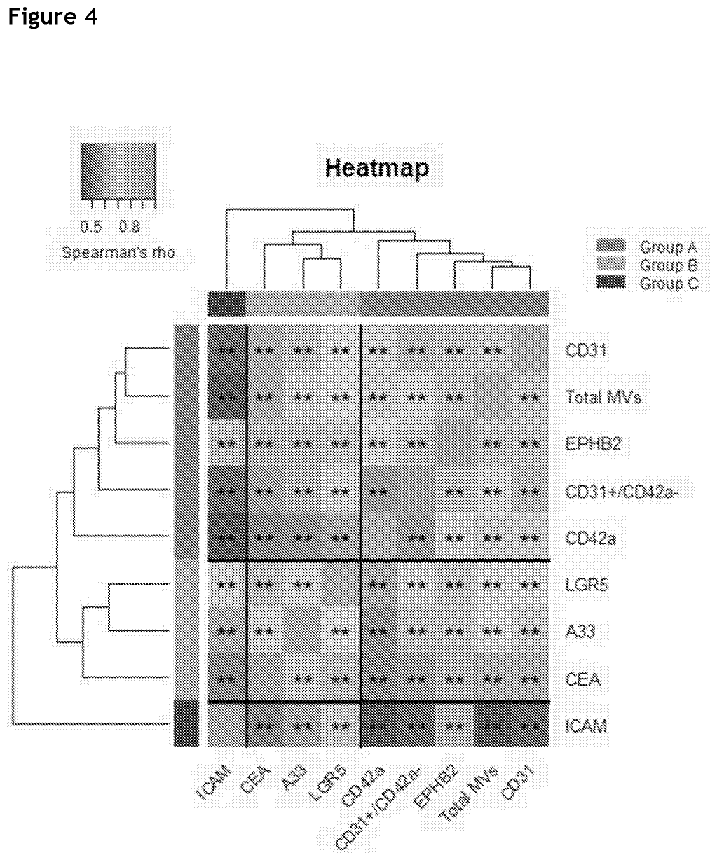

Plasma levels of several MVs positive for known serum CRC biomarkers were measured. The markers include: CEA, A33, LGR5, EPHB2, ICAM, CD31, CD42a and CD31+/CD42a−. Table 3 summarises the means and SD in case of parametric distribution, and median and range in case of non-parametric distribution. The p values was generated by the appropriate ANOVA test for multiple groups and t-test to compare the unpaired groups separately (Table 3 and FIG. 4).

TABLE 3
The levels of selected colorectal cancer markers identified on microvesicles

| | Healthy Controls | Benign CR polyps | CRC | p value |
|---|---|---|---|---|
| | (n = −17)† | (n = −16)† | (n = −19)† | (ANOVA) |
| CEA MVs/μL:**** median (range) | 8 (1-40) | 29 (7-136) | 68 (10-625) | <0.0001 |
| A33 MVs/μL:**** mean (SD) | 40 (29) | 127 (87) | 182 (121) | <0.0001 |
| LGR5 MVs/μL:** mean (SD) | 63 (54) | 232 (147) | 271 (207) | 0.001 |
| EPHB2 MVs/μL:*** mean (SD) | 71 (49) | 324 (171) | 276 (224) | 0.0002 |
| ICAM MVs/μL:** median (range) | 14 (2-87) | 63 (5-1303) | 54 (6-376) | 0.008 |
| CD31 MVs/μL:*** mean (SD) | 39 (28) | 218 (175) | 209 (150) | 0.0003 |
| CD42a MVs/μL:*** median (range) | 18 (5-155) | 198 (27-959) | 102 (9-492) | 0.0002 |
| CD31+/CD42a− MVs/μL:**** mean (SD) | 25 (20) | 110 (71) | 135 (81) | <0.0001 |

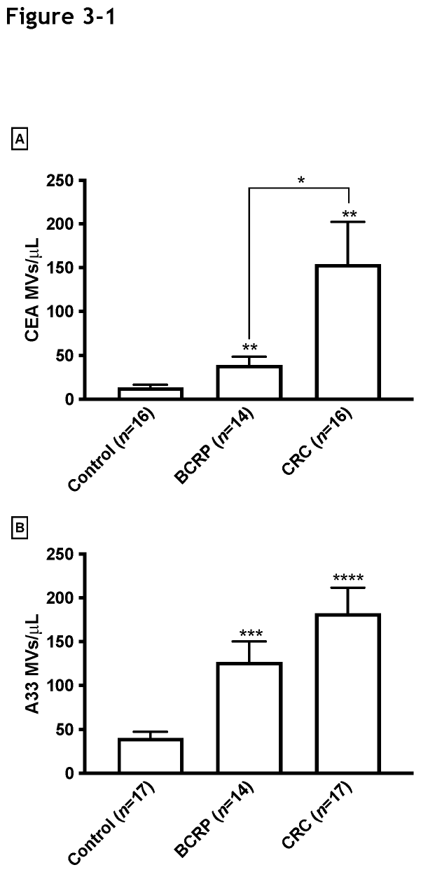

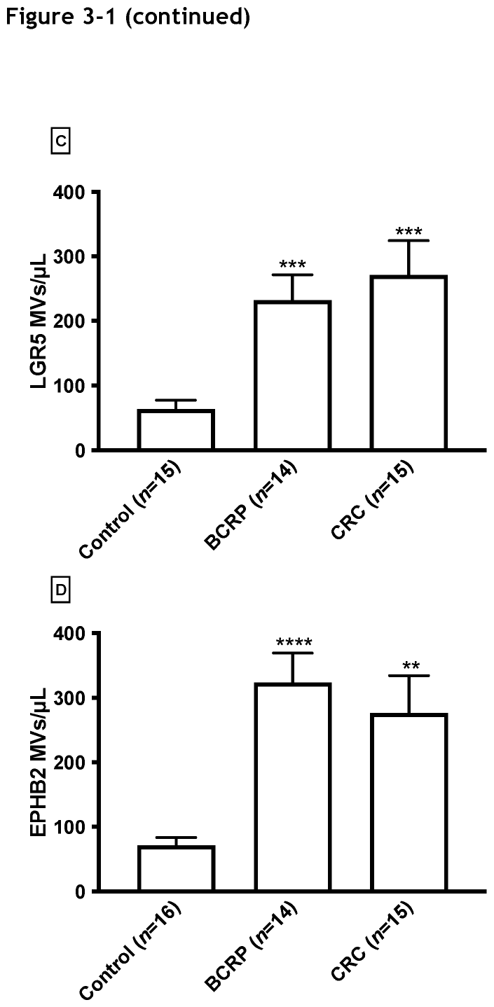

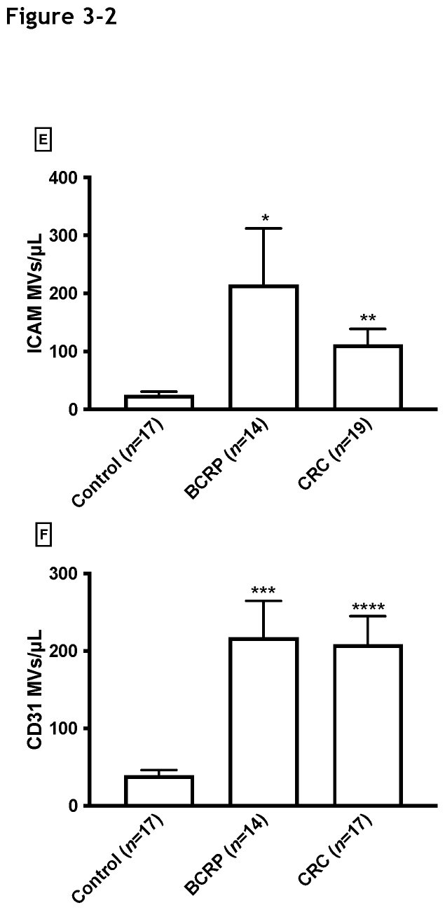

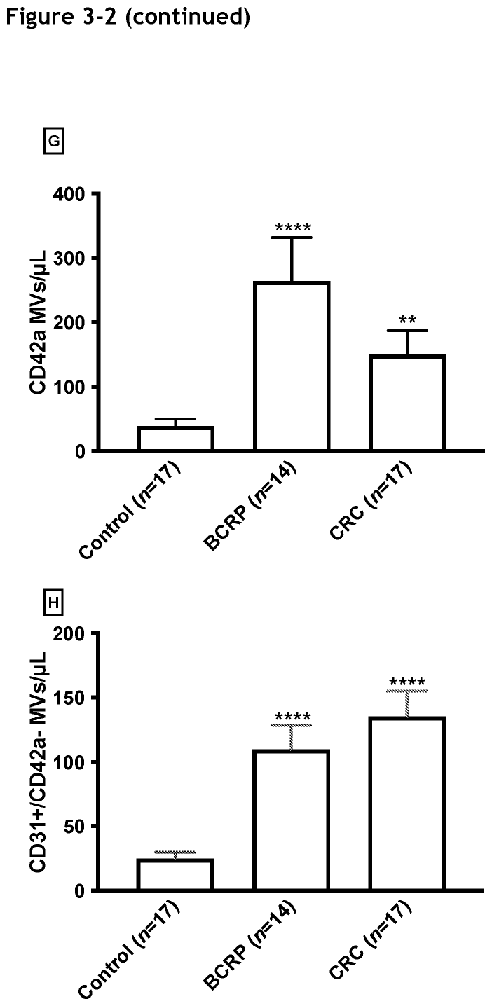

CEA: carcinoembryonic antigen. CR: colorectal. CRC: colorectal cancer. EPHB2: ephrin type-B receptor 2. ICAM: intercellular adhesion molecule. LGR5: leucine rich repeat containing G protein coupled receptor 5. MVs: microvesicles. SD: standard deviation.
*p < 0.05.
**p < 0.01.
***p < 0.001.
****p < 0.000.
†Sample size is variable please refer to FIG. 3 for individual markers sample size.

### Factorial Analysis and Correlation Matrix

In order to achieve the most accurate diagnostic value of the multiple MVs markers, factorial analysis was performed. All the markers were subjected to principle component analysis (PCA) using SPSS version 25. Prior to performing PCA, the suitability of data for factor analysis was assessed by a correlation matrix and cluster grouping of the plasma MVs markers. Table 4 and FIG. 4 summarise the spearman rho values and the statistical significance of each correlation coefficient. Dendrogram resulted into 3 clusters: 1) CD31, total MVs, EPHB2, CD31+/CD42a− and CD42a; 2) LGR5, A33 and CEA; and 3) ICAM. Most correlation coefficients (Spearman's roh) were above 0.3. The Kaiser-Meyer-Oklin value was 0.64, exceeding the recommended value of 0.6 (Kaiser H F. A second generation little jiffy. Psychometrika 1970; 35(4):401-415; Kaiser H F; An index of factorial simplicity. Psychometrika 1974; 39(1):31-36). Barlett's Test of Sphericity (Bartlett M S. A Note on the Multiplying Factors for Various χ<sup>2</sup> Approximations. Journal of the Royal Statistical Society. Series B (Methodological) 1954; 16(2):296-298) was statistically significant (p<0.0001), supporting the factorability of the correlation matrix.

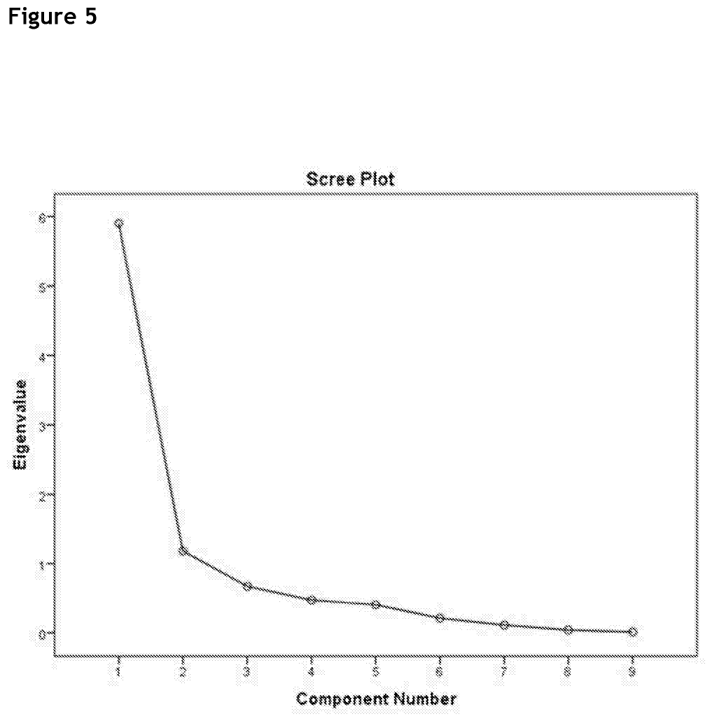

PCA confirmed the presence of two components with eigenvalues exceeding 1, explaining 66% and 13% of the variance respectively. Coefficients from Component Pattern and Structure Matrix are summaries in Table 5. An inspection of the screeplot revealed a clear break after the second component (FIG. 5). Using Catell's scree test, it was decided to use the 2 component factors for further Investigation (Cattell R B. The Scree Test For The Number Of Factors. Multivariate Behavioral Research 1966; 1(2):245-276. There was a positive correlation between the two factors (r=0.47). Component score coefficient matrix for components 1 and 2 are shown in Table 6. Although, for diagnostic proposes component score coefficients can be used to compute the values levels of markers in the component factors for a particular patient, an easier method described by Grice (Grice J W. Computing and evaluating factor scores. Psychol Methods 2001; 6(4):430-50) was adopted. This method uses values generated from pattern matrix (Table 4). It assigns a value of 1 to the coefficients for variables with loadings greater than 0.4 and zero to the coefficients for variables with loading equal to or less than 0.4. Computation of component factor for patients is explained below (in section titled “Component factor 1 as a diagnostic tool for colorectal benign polyps and cancer”).

TABLE 5
Pattern and structure matrix for principal component analysis with
Oblimin rotation with Kaiser normalisation of two factor solution

| | Pattern coefficients | | Structure coefficients | | |
|---|---|---|---|---|---|
| | Component 1 | Component 2 | Component 1 | Component 2 | Communalities |
| CD31 | 1.002 | | .964 | .389 | 0.935 |
| CD42a | .964 | | .896 | .307 | 0.820 |
| Total MVs | .862 | | .870 | .420 | 0.757 |
| CD31+/CD42a− | .799 | | .838 | .456 | 0.707 |
| EPHB2 | .618 | .432 | .820 | .721 | 0.818 |
| ICAM | .541 | .348 | .703 | .601 | 0.589 |
| A33 | | .940 | .741 | .878 | 0.911 |
| CEA | .305 | .700 | | .860 | 0.762 |
| LGR5 | .423 | .681 | .632 | .842 | 0.782 |

TABLE 6
Component score coefficient matrix

| | Component 1 | Component 2 |
|---|---|---|
| Total MVs | .209 | −.055 |
| CEA | −.112 | .465 |
| A33 | .053 | .296 |
| LGR5 | .023 | .314 |
| EPHB2 | .119 | .162 |
| ICAM | .107 | .128 |
| CD31 | .251 | −.112 |
| CD42a | .246 | −.140 |
| CD31+/CD42a− | .189 | −.019 |

### Component Factor 1 as a Diagnostic Tool for Colorectal Benin Polyps and Cancer

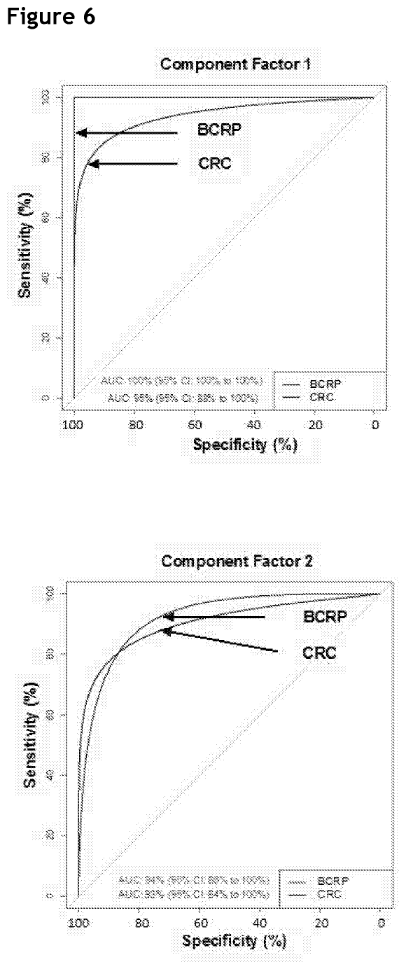

Component factor 1 was able to significantly (p<0.0001) diagnose BCRP and CRC with area under the ROC of 100 (95% CI: 100% to 100%) and 95% (95% CI: 88% to 100%) respectively (Table 7 and FIG. 6). Similarly, component factor 2 was able to significantly (p<0.0001) diagnose BCRP and CRC with AUC of 94% (95% CI: 86% to 100%) and 93% (95% CI: 84% to 100%) respectively (Table 7 and FIG. 6).

Values for component factors 1 and two and cut-off points were computed using Grice method. The following equation was used: F1=b11X1+b12X2+b13X3 etc. where value 1 was assigned to b-coefficients for variables with loadings greater than 0.4, and 0 for variables with loadings equal to or less than 0.4. Component factor 1 included total plasma MVs, and MVs positive for CD31, CD42a, CD31+/CD42a−, EPHB2, ICAM and LGR5 (Table 5). Values from MVs positive for CEA were excluded because its pattern coefficient was less than 0.4. Similarly, component factor 2 included MVs positive for EPHB2, A33, CEA, and LGR5. MVs positive for ICAM was excluded because its coefficient was less than 0.4 (Table 5). AUC, 95% CI, cut-off points, sensitivity, specificity, PPV and NPV for the diagnosis of BCRP and CRC using component factors 1 and 2 are summaries in Table 7.

Component factor 1 provided more accurate diagnosis. Indeed, a cut-off point of 439 MVs/μL have a 100% sensitivity, 100% NPV, 50% specificity and 78% PPV.

TABLE 7
Diagnostic accuracy of component factors 1 and 2 for the diagnosis of BCRP and CRC

| | AUC | 95% CI | Cut-off point | Sensitivity | Specificity | PPV | NPV | p value |
|---|---|---|---|---|---|---|---|---|
| BCRP | | | | | | | | |
| Component factor 1 | 1 | 1 to 1 | >761 MVs/μL | 100% | 100% | 93% | 100% | <0.0001 |
| Component factor 2 | 0.94 | 0.86 to 1 | >222 MVs/μL | 100% | 63% | 70% | 100% | <0.0001 |
| CRC | | | | | | | | |
| Component factor 1 | 0.95 | 0.88 to 1 | >439 MVs/μL | 100% | 56% | 65% | 100% | <0.0001 |
| Component factor 2 | 0.93 | 0.84 to 1 | >157 MVs/μL | 100% | 50% | 65% | 100% | <0.0001 |

AUC: area under the receiver operating curve. BCRP: benign colorectal polyps. CRC: colorectal cancer. CI: confidence interval. MVs: microvesicles. NPV: negative predictive value. PPV: positive predictive value.

### Example 4: Distinguishing Between Benign Colorectal Polyps and Cancer

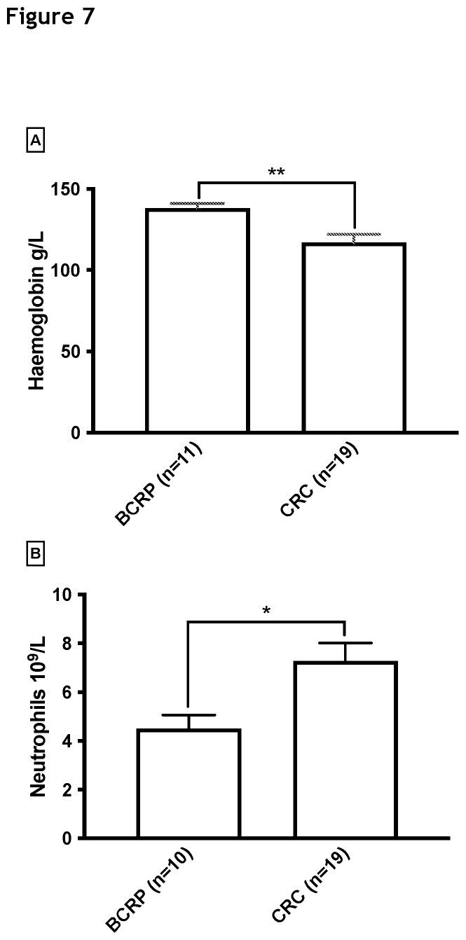

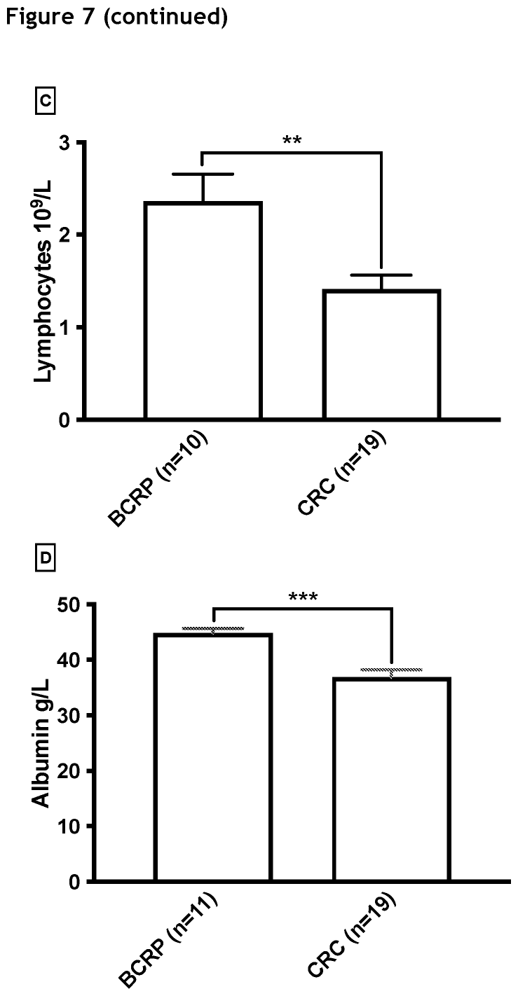

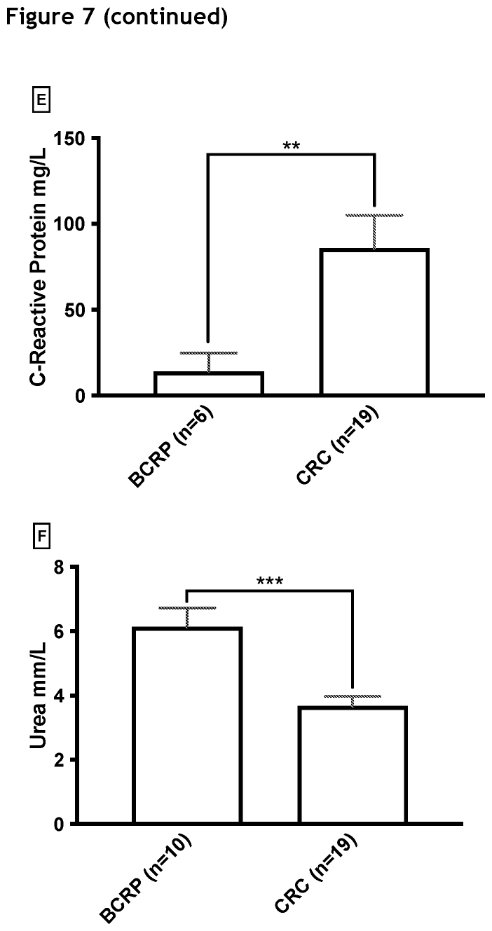

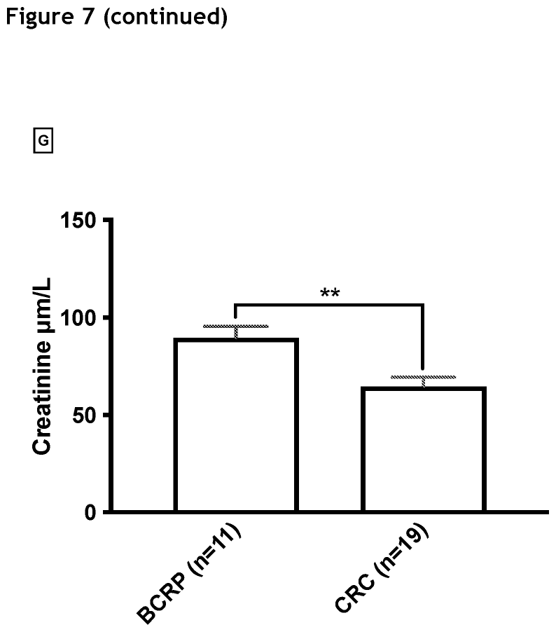

To distinguish between BCRP and CRC the potential predictive values of routine blood tests and other markers were examined. Those with values significantly different between BCRP and CRC using unpaired t-test were considered for further analysis. The blood levels of haemoglobin, lymphocytes, albumen, urea, and creatinine were significantly lower in CRC (Table 1 and FIG. 7). However, neutrophils count and CRP were statistically significantly higher in the CRC patients (Table 1, FIG. 7). Furthermore, the plasma level of MVs positive for CEA was significantly higher in patients with CRC (Table 3 and FIG. 3).

### Diagnostic Accuracy of Routine Blood and Positive CEA Microvesicles for the Diagnosis of Colorectal Cancer

AUC analysis was performed to establish the diagnostic accuracy of haemoglobin, neutrophils, lymphocytes, albumen, CRP, urea, creatinine and MVs positive for CEA. Table 8 summarises the results for this analysis. Albumen and haemoglobin appear to have the most accurate and significant values. Cut-off points with highest specificity and PPV were used. This is to most accurately predict CRC from BCRP in patients who have raised total MVs or component factor 1.

TABLE 8
Diagnostic accuracy of routine blood and MVs CEA+ for distinguishing between patients
suffering from BCRP and CRC. The control group are results from patients with BCRP.

| | AUC | 95% CI | Cut-off point | Sensitivity | Specificity | PPV | NPV | p value |
|---|---|---|---|---|---|---|---|---|
| Haemoglobin (g/L) | 0.87 | 0.73 to 1 | <124.5 | 68% | 100% | 100% | 63% | 0.001 |
| Neutrophils (10₉/L) | 0.76 | 0.59 to 0.93 | >7.7 | 47% | 100% | 100% | 50% | 0.02 |
| Lymphocytes (10₉/L) | 0.81 | 0.64 to 0.97 | <1.16 | 47% | 100% | 100% | 50% | 0.008 |
| Albumen (g/L) | 0.9 | 0.78 to 1.01 | <39.5 | 63% | 100% | 100% | 61% | 0.0004 |
| CRP (mg/L) | 0.85 | 0.68 to 1.03 | >72.75 | 47% | 100% | 100% | 38% | 0.01 |
| Urea (mmol/L) | 0.85 | 0.71 to 1 | <3.85 | 58% | 100% | 100% | 56% | 0.002 |
| Creatinine (μmol/L) | 0.75 | 0.69 to 0.98 | <63.5 | 53% | 100% | 100% | 55% | 0.003 |
| CEA (MVs/μL) | 0.75 | 0.57 to 0.03 | >221 | 25% | 100% | 100% | 54% | 0.02 |

AUC: area under the receiver operating curve. BCRP: benign colorectal polyps. CEA: carcinoembryonic antigen. CRC: colorectal cancer. CRP: C-reactive protein. CI: confidence interval. MVs: microvesicles. NPV: negative predictive value. PPV: positive predictive value.

Prediction of Colorectal Cancer with Logistic Regression Model

Logistic regression was performed to assess the impact of a number of factors on the likelihood CRC in patients who are positive for component factor 1. Results from patients with BCRP were used as the reference category. A number of eight independent variables were considered (haemoglobin, neutrophils, lymphocytes, albumin, urea, creatinine and MVs positive for CEA).

Logistic regression outcome with OR, 95% CI, constant and b-coefficient is summarised in Table 9. While low haemoglobin, lymphocytes, albumen, urea and creatinine, and high CRP levels can significantly predict CRC, OR associated with high neutrophils levels are statistically insignificant.

TABLE 9
Logistic regression analysis to ass the probability
of CRC from routine blood tests and MVs positive
for CEA showing OR and significance.

| | | 95% CI | | | Con- | p |
|---|---|---|---|---|---|---|
| | OR | Lower | Upper | B | stant | value |
| Haemoglobin (g/L) | 0.93 | 0.88 | 0.99 | −0.71 | 9.59 | 0.02 |
| Neutrophils (10₉/L) | 1.17 | 0.89 | 1.54 | 0.16 | −0.46 | 0.27 |
| Lymphocytes (10₉/L) | 0.21 | 0.60 | 0.75 | −1.56 | 3.50 | 0.01 |
| Albumin (g/L) | 0.62 | 0.42 | 0.90 | −0.49 | 20.87 | 0.01 |
| CRP (mg/L) | 1.05 | 0.99 | 1.09 | 0.04 | −0.35 | 0.06 |
| Urea (mmol/L) | 0.33 | 0.14 | 0.79 | −1.10 | 5.86 | 0.01 |
| Creatinine (μmol/L) | 0.94 | 0.90 | 0.99 | −0.06 | 5.20 | 0.01 |
| CEA (MVs/μL) | 1 | 1 | 1 | 0 | −0.86 | 0.14 |

CEA: carcinoembryonic antigen. CRC: colorectal cancer. CRP: C-reactive protein. CI: confidence interval. MVs: microvesicles.

$$Equation 9. Probability of colorectal cancer against benign \\ colorectal polyps = \frac{1}{( 1 + Exp ( - constant + Haemoglobin \times b ) ) )}$$

Correlation matrix was performed to assess the multicollinearity of these variables. There was strong correlations between theses variables. Due to multicollinearity, multivariate analysis to predict the probability of CRC was inappropriate. However, a logistic regression equation can be adopted to predict CRC using constants generated from the univariate analysis (Equation 9). Haemoglobin or albumen demonstrate the highest AUC value.

Prediction of Colorectal Cancer with Venn Diagram

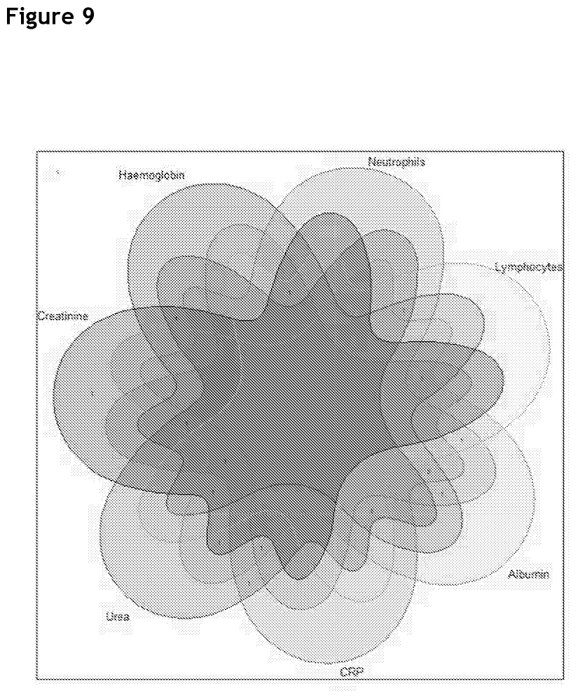

In order to distinguish BCRP from CRC, values from routine blood with cut-off point of high specificity, as specified in Table 8, were chosen to identify the number of patients who are positive for CRC. These markers include: haemoglobin, neutrophils, lymphocytes, albumin, CRP, urea and creatinine. Venn diagram (FIG. 9) and Table 10 summaries the number of patients positive for multiple markers. There was not any patient who tested positive for all the blood markers, which suggest that although these markers are individually significantly different in CRC patients, in combination they may not be.

TABLE 10
Individual CRC patients results of routine blood of significance predictive values.

| CRC patients | Haemoglobin | Neutrophils | Lymphocytes | Albumen | CRP | Urea | Creatinine |
|---|---|---|---|---|---|---|---|
| P1 | 0 | 1 | 1 | 1 | 0 | 0 | 0 |
| P2 | 1 | 0 | 1 | 1 | 1 | 1 | 0 |
| P3 | 1 | 0 | 0 | 1 | 1 | 1 | 1 |
| P4 | 0 | 0 | 0 | 0 | 0 | 0 | 0 |
| P5 | 0 | 0 | 0 | 1 | 0 | 1 | 1 |
| P6 | 1 | 1 | 1 | 1 | 0 | 1 | 1 |
| P7 | 1 | 1 | 0 | 1 | 1 | 0 | 0 |
| P8 | 1 | 1 | 1 | 0 | 1 | 0 | 1 |
| P9 | 0 | 0 | 0 | 0 | 1 | 1 | 0 |
| P10 | 1 | 1 | 1 | 0 | 0 | 1 | 0 |
| P11 | 1 | 0 | 1 | 1 | 1 | 1 | 1 |
| P12 | 1 | 1 | 0 | 1 | 0 | 0 | 0 |
| P13 | 1 | 0 | 0 | 0 | 0 | 1 | 1 |
| P14 | 0 | 0 | 0 | 0 | 0 | 0 | 1 |
| P15 | 1 | 1 | 1 | 0 | 0 | 1 | 1 |
| P16 | 1 | 1 | 0 | 1 | 1 | 0 | 0 |
| P17 | 1 | 0 | 1 | 1 | 1 | 0 | 1 |
| P18 | 0 | 1 | 0 | 1 | 1 | 1 | 0 |
| P19 | 1 | 0 | 1 | 1 | 0 | 1 | 1 |

CRC: colorectal cancer. CRP: C-reactive protein. P: patient. 1: positive. 0: negative.

### Example 5: Plasma Level of Total Microvesicles in Benign Colorectal Polyps, Colorectal Cancer and Other Bowel Conditions

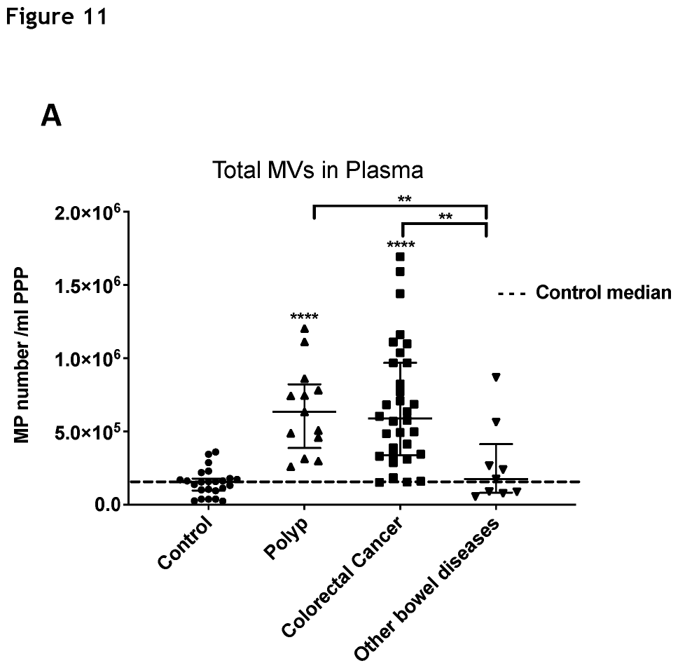

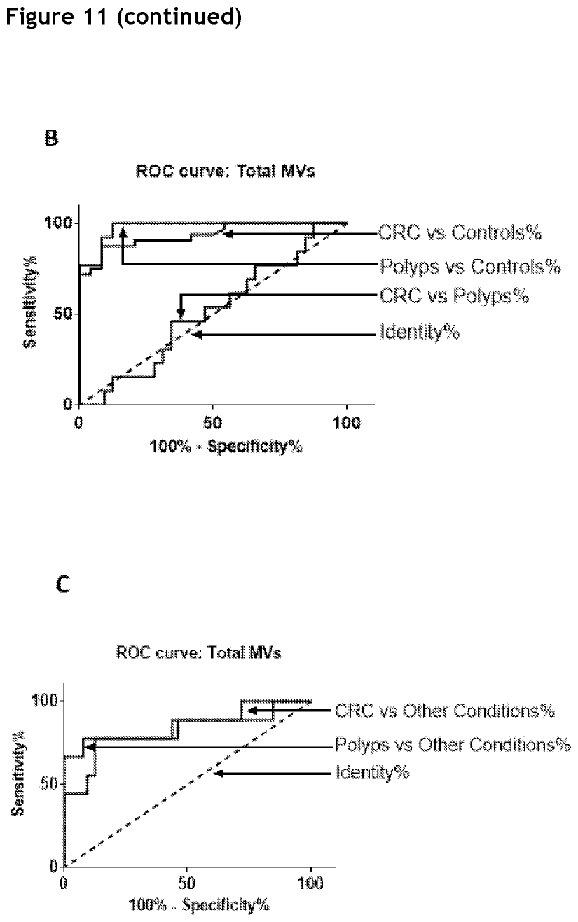

Patients with CRC and polyps had significantly higher total plasma MV counts of median 588888 (152354-1692662) MVs/ml (n=32; p<0.0001) and 633362 (259592-1203052) MVs/ml plasma (n=13; p<0.0001), respectively, compared to healthy controls with a median of 155651 (24293-359048) MVs/ml (n=24; FIG. 11). ROC curve analyses showed high sensitivity (87.1% and 92.31% with the threshold at 287358 MVs/ml and 292058 MVs/ml) and for total MVs in significantly distinguishing patients with CRC and polyps, respectively, from healthy controls. The CRC and polyp groups had significantly higher levels of total MVs compared to patients with other bowel conditions such as IBD and diverticular disease (174603 (54486-868219) MVs/ml; n=9, p<0.01 for both groups). No significant difference was observed between the CRC group and the polyp group and between healthy controls and patients with other bowel conditions. There was moderately high sensitivity (77.78% with threshold at 280986 MVs/ml) and high overall accuracy (AUC=0.8462; p=0.0068) when using total MVs to distinguish the polyp group from patients with other bowel conditions. The sensitivity was 55.56% with the threshold at 179790 MVs/ml but overall accuracy remained very high (AUC=0.8333 with p=0.0025) when using total MVs to distinguish patients with CRC from other bowel conditions.

### Example 6: Plasma Levels of Microvesicles Positive for A33

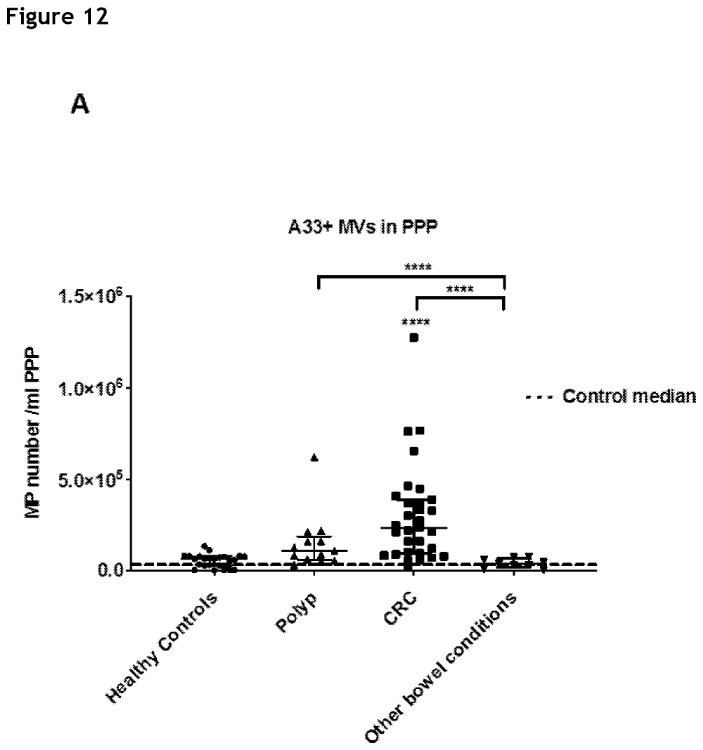

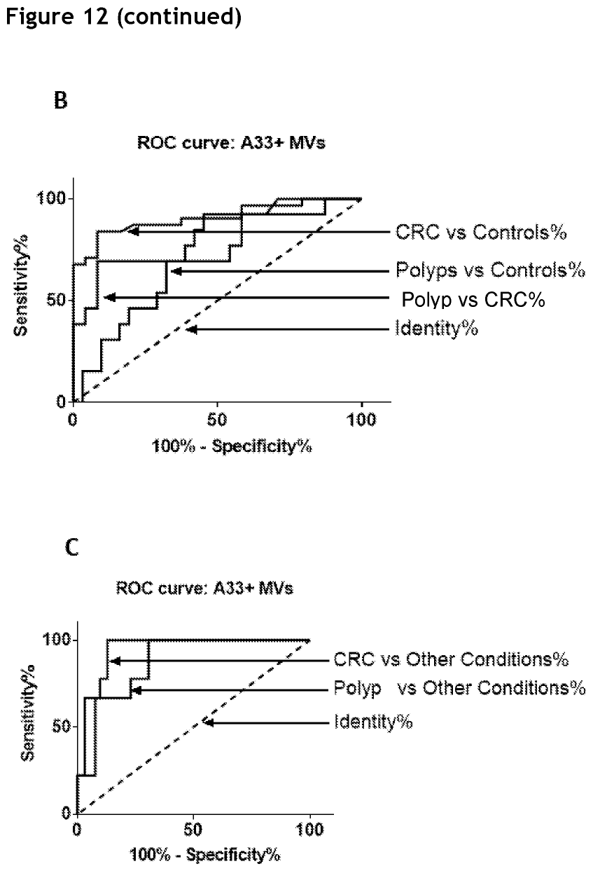

Patients with colorectal cancer had significantly elevated levels of A33+ MVs at 236686 (19782-1273474) MVs/ml (n=31) compared to healthy controls (65476 (3470-134127) MVs/ml; n=24; p<0.0001) and patients with other bowel conditions (38472 (3470-76291) MVs/ml; n=9; p<0.01; FIG. 12). Such an elevation was not seen in patients in the polyp group (11106 (31928-620803) MVs/ml; n=13). ROC curve analyses revealed high sensitivity (83.87% with the threshold at 83494 MVs/ml at >90% specificity) and accuracy (AUC=0.9066, p<0.0001) when using A33+ MVs to determine CRC patients from the control group. A significant difference in A33+ MVs was not seen between the polyp and other bowel conditions groups compared to healthy controls. Both sensitivity (30.77% at >90% specificity) and accuracy (AUC=0.7171; p=0.0244) were low when using A33+ MVs to distinguish CRC patients from the polyp group. Sensitivity was 77.78% (at a threshold of 66286 MVs/ml; AUC=0.9462, p=0.0001) when using A33+ MVs to detect CRC patients from patients with other bowel conditions. Sensitivity was only 66.67% at >90% specificity at a threshold of 48227 MVs/ml (AUC=0.8718, p=0.0037) when using A33+ MVs to detect precancerous patients from patients with other bowel conditions.

### Example 7: Plasma Levels of Microvesicles Positive for CEA

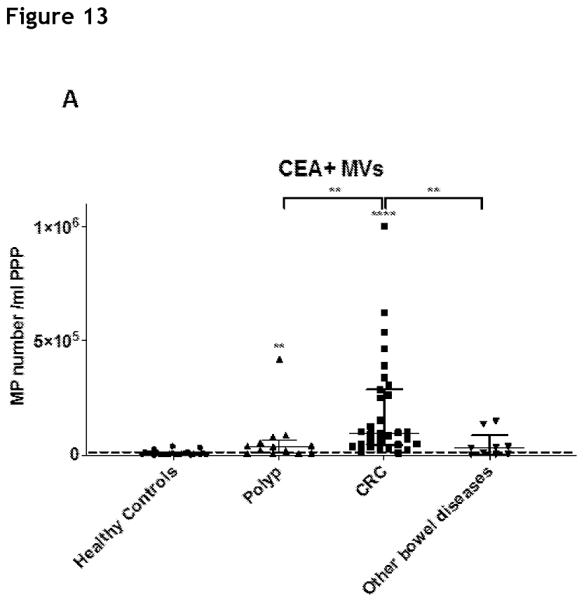

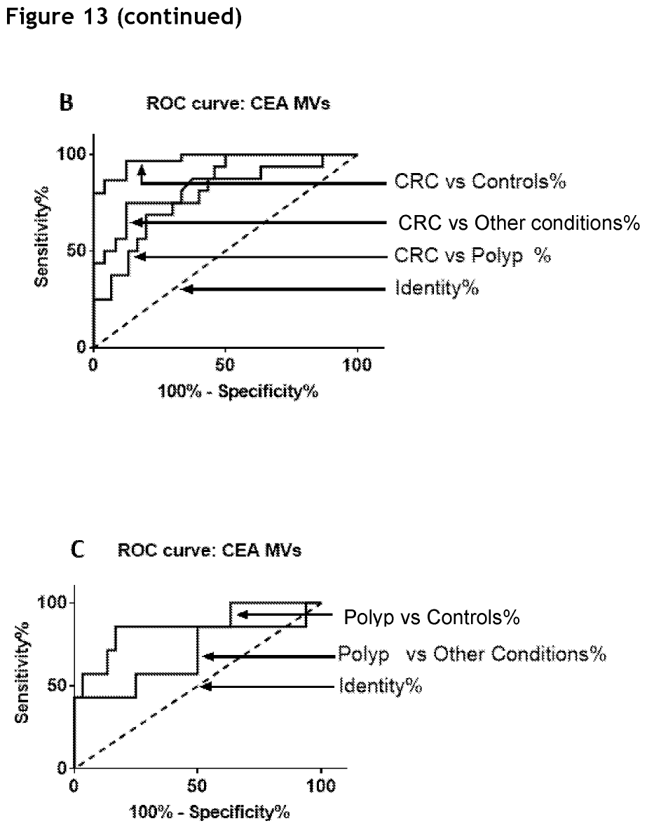

CEA+ MVs were significantly elevated in both the CRC group with 94219 (10411-1001565) MVs/ml (n=31; p<0.0001) and polyp group with 38522 (6847-418300) MVs/ml (n=13; p<0.01) compared to healthy controls who had 6601 (694-39910) MVs/ml (n=24; FIG. 13). This elevation was not seen in the other bowel conditions group (31299 (2429-148205) MVs/ml; n=7). CEA+ MVs were significantly higher in CRC than in other bowel conditions (p<0.05). ROC curve analyses showed high sensitivity (86.67% sensitivity with the threshold at 31061 MVs/ml at >90% specificity) and high accuracy (AUC=0.9736, p<0.0001) for detecting colorectal patients from the control group. Sensitivity was poor (56.25% at >90% specificity) though overall accuracy was high (AUC=0.8659, p=0.0001) when using this MV subset to detect patients with polyp from healthy controls. Sensitivity was poor (37.5% at specificity>90%; AUC=0.775, p>0.0023) when using CEA+ MVs to distinguish CRC patients from the polyp group. No significant difference was observed between the three disease groups.

### Example 8: Plasma Levels of Microvesicles Positive for LGR5

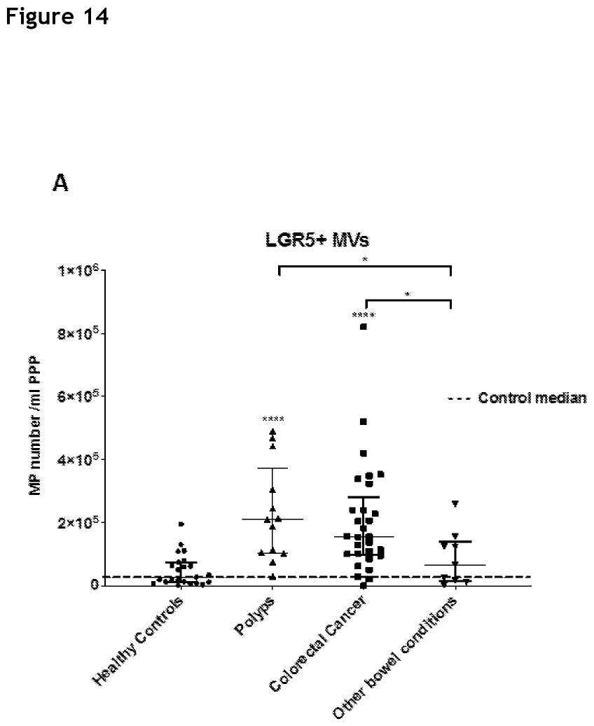

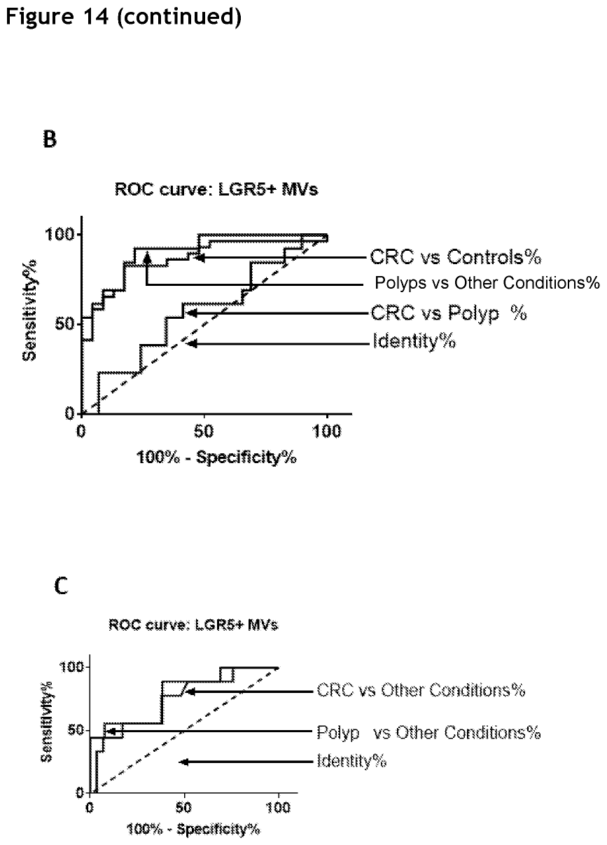

LGR5+ MVs were significantly elevated in CRC with 155130 (347-820284) MVs/ml (n=29; p<0.001) and precancerous patients with 209830 (29669-489684) MVs/ml (n=13; p<0.0001) compared to healthy controls with 27070 (794-194694) MVs/ml (n=23; FIG. 14). Sensitivity was moderate when using LGR5+ MVs to distinguish CRC (65.52% with threshold at 112405 MVs/ml; AUC=0.8621, p=0<0001) and precancerous patients (69.23% with threshold at 111402 MVs/ml; AUC=0.9097, p<0.0001) from healthy controls. Levels of LGR5+ MVs were not significantly different between the polyp and cancer groups. The CRC and polyp groups had higher levels of LGR5+ MVs compared to patients with other bowel conditions (65858 (952.4-259194) MVs/ml; n=9), though only the difference for the latter showed statistical significance (p<0.05). LGR5+MV levels showed a low sensitivity of 44.44% at >90% specificity for detecting both CRC (with a threshold at 26752 MVs/ml; AUC=0.7375, p=0.0333) and precancerous patients (with a threshold at 47633 MVs/ml; AUC=0.7863, p=0.0253) from other bowel conditions.

### Example 9: Plasma Levels of Microvesicles Positive for EPhB2+

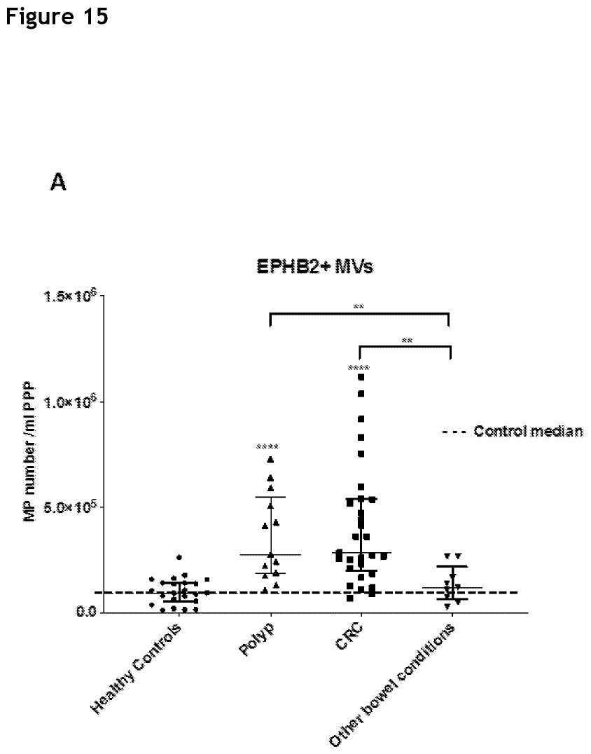

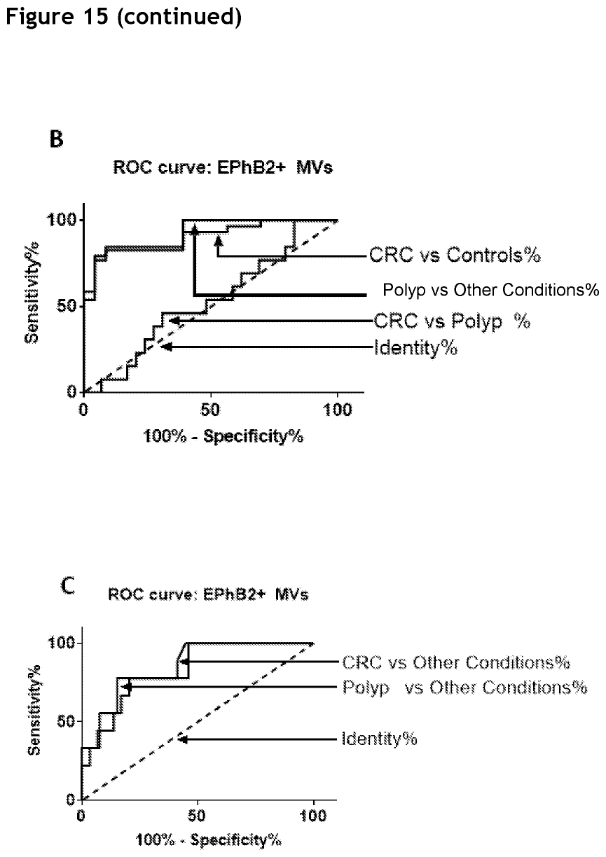

EPhB2+ MVs showed a similar profile to LGR5+. Both CRC (283320 (66980-1116945) MVs/ml; n=29, p<0.0001) and polyp (275209 (106891-728702) MVs/ml; n=13, p<0.001) groups had significantly elevated EPhB2+ MVs compared to healthy controls (93356 (11279-261746) MVs/ml (n=23; p<0.0001 and p<0.001, respectively; FIG. 15) and patients with other bowel conditions (118571 (26376-267574) MVs/ml; n=9; p<0.05 and p<0.05, respectively). These markers showed high sensitivities and accuracies when used to identify CRC (82.76% sensitivity with the threshold at 163994 MVs/ml at >90% sensitivity, AUC=0.904, p<0.0001) and precancerous patients (84.62% with the threshold also at 169693 MVs/ml, AUC=0.9231, p<0.0001) from control subjects. There was no difference in levels between the CRC and polyp groups. These markers displayed moderate sensitivity in distinguishing CRC (44.44% sensitivity at >90% specificity at threshold 109182 MVs/ml; AUC: 0.8372, p=0.0025) and polyps (55.56% at >90% specificity at threshold 124878 MVs/ml; AUC=0.8462, p=0.0068) from other bowel conditions, with the identification of the polyp group faring slightly better than the CRC group.

### Example 10: Plasma Levels of Microvesicles Positive for Cytokeratin

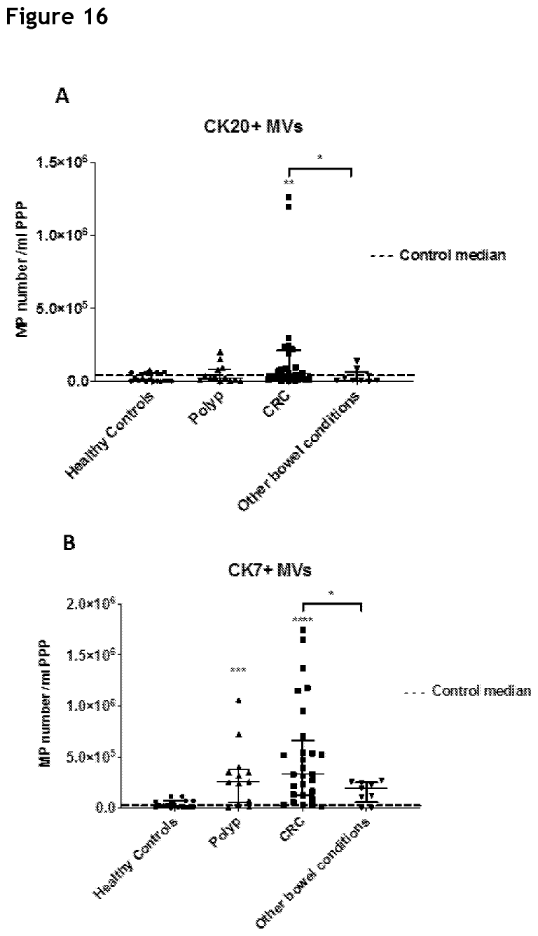

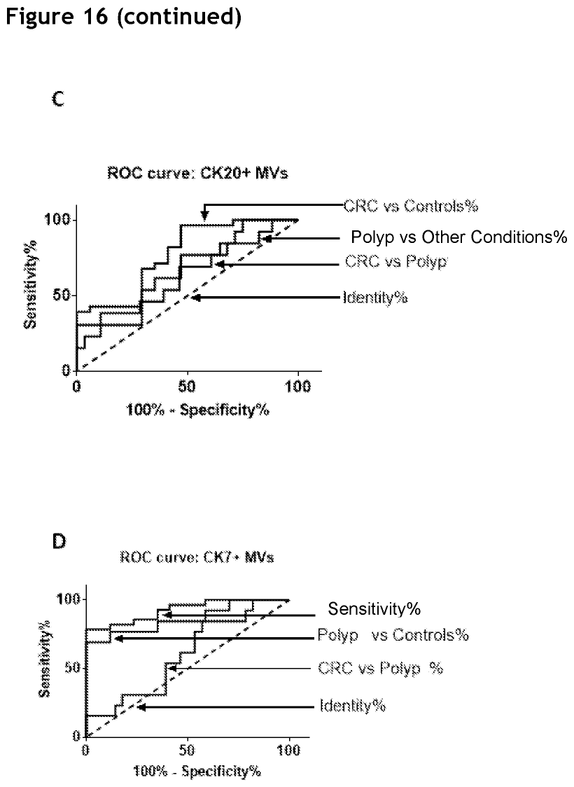

MVs with cytokeratin were also studied. Cytokeratin 20+ MVs were significantly elevated (56729 (4586-1659168) MVs/ml) in colorectal cancer patients compared to healthy controls (7937 (1310-74683) MVs/ml; p<0.01), but not compared to patients with polyp (30627 (2948-202888) MVs/ml) and other bowel conditions (7937 (794-88113) MVs/ml; FIG. 16). Cytokeratin 20+MV levels were not significantly different from the control group. Cytokeratin 7+ MVs were also significantly elevated in both the CRC (336402 (18343-1743936) MVs/ml; p<0.001) and polyp (259153 (11137-1061286) MVs/ml; p<0.001) groups compared to healthy controls (31446 (3931-118903) MVs/ml) though there was no difference between any of the disease groups. There was no elevation of Cytokeratin 7+MV levels in patients with other bowel conditions (125127 (4444-246978) MVs/ml) compared to healthy controls. ROC curve analyses showed that Cytokeratin 20+ MVs had low sensitivity (44.44% at a threshold of 67477 MVs/ml; AUC=0.7778, p=0.0021) in detecting colorectal cancer from healthy controls. Cytokeratin 7+ MVs fared better as sensitivity was 77.78% when used to distinguish both colorectal cancer patients (at a threshold of 117593 MVs/ml; AUC=0.9237; p<0.0001) and 75% when distinguishing patients with polyp (at a threshold of 117593 MVs/ml; AUC=0.8897; p=0.0001) from healthy control subjects. Levels of MVs between the cancer and polyp group were not significantly different.

### Example 11: Plasma Levels of Cytokeratin 20+/Cytokeratin 7− Microvesicles

The level of cytokeratin 20+/cytokeratin 7− (CK20+/CK7−) MV subpopulation was significantly elevated in colorectal cancer (21580 (0-271481) MVs/ml; n=27) compared to both the polyp (5568 (0-84837) MVs/ml; n=16; P<0.01) and the other bowel conditions group (1965 (634-40617) MVs/ml; n=7; P<0.05). These markers showed 37.5% sensitivity at a threshold of 3931 MVs/ml (AUC=0.8044; p<0.0010) when used to detect CRC patients from patients with polyp and 57.14% sensitivity at a threshold of 3831 MVs/ml (AUC=0.8095; p=0.0127). No significant difference was observed between healthy controls and the cancer group possibly due to the low levels of these markers present in plasma.

### Example 12: Plasma Levels of Microvesicles Positive for HLA-DR

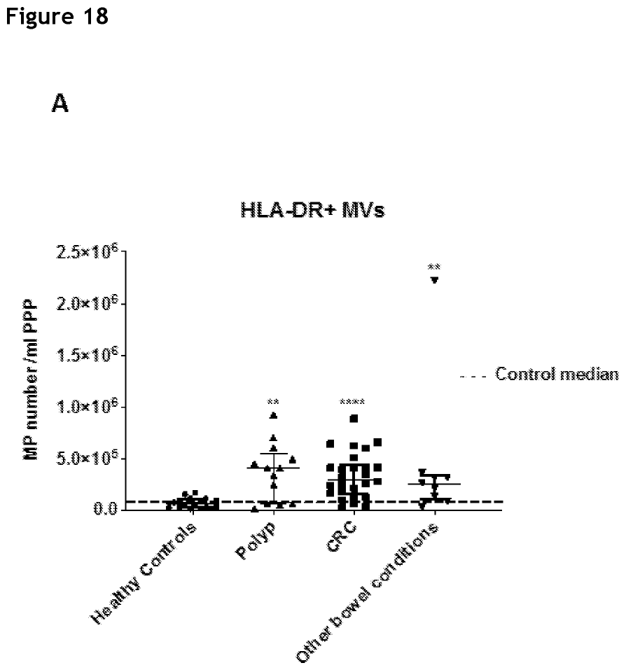

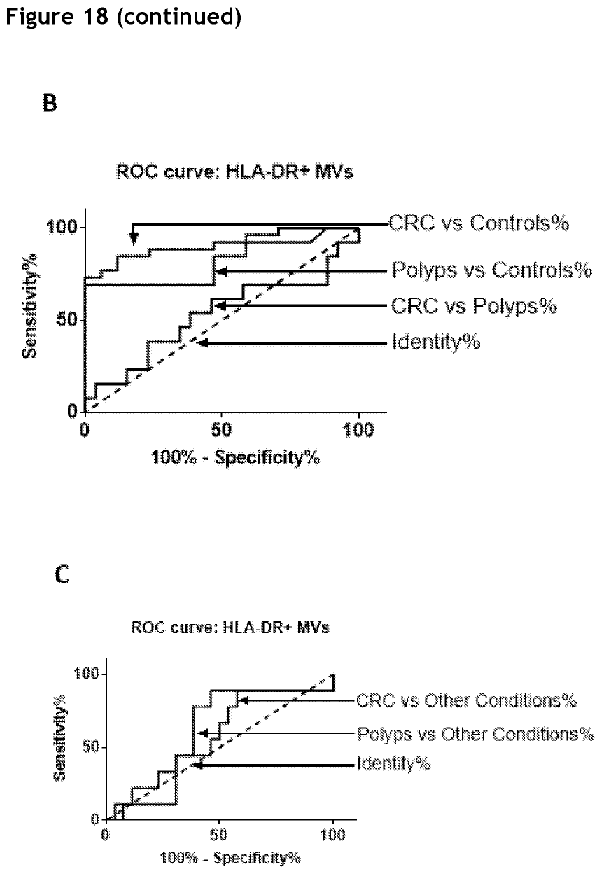

HLA-DR+MV levels were higher in both colorectal cancer (277769 (27187-883422) MVs/ml; p<0.001; n=25) and polyp (406007 (16705-2217236) MVs/ml; P<0.001; n=16) compared to healthy controls (56984 (12120-169520) MVs/ml; n=17; FIG. 18). There was no significant difference observed between patients with other bowel conditions and healthy controls as well as between the polyp and colorectal cancer groups. ROC analyses showed these MVs had moderately high sensitivity in detecting both colorectal cancer (76% sensitivity at a threshold of 160765 MVs/ml; AUC=0.9082, p<0.0001) and polyp (74% sensitivity at a threshold of 161508 MVs/ml; AUC=0.8511, p<0.0006) from healthy control subjects.

### Example 13: Plasma Levels of Microvesicles Positive for CD147

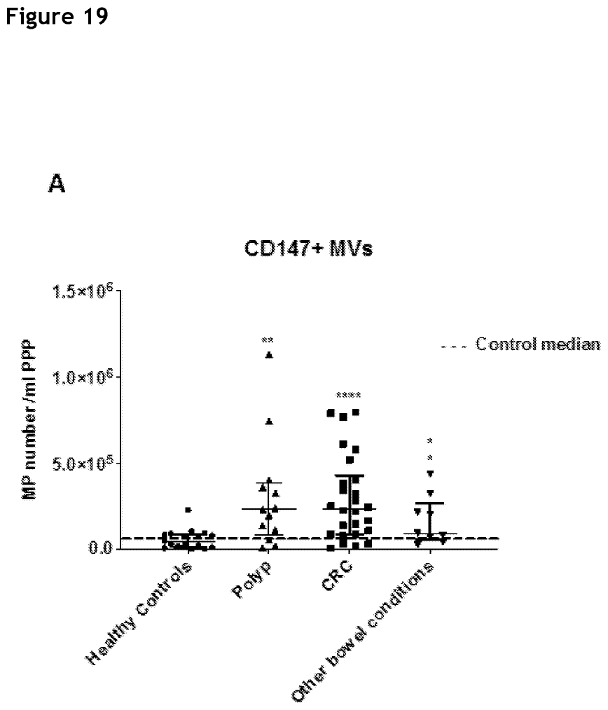

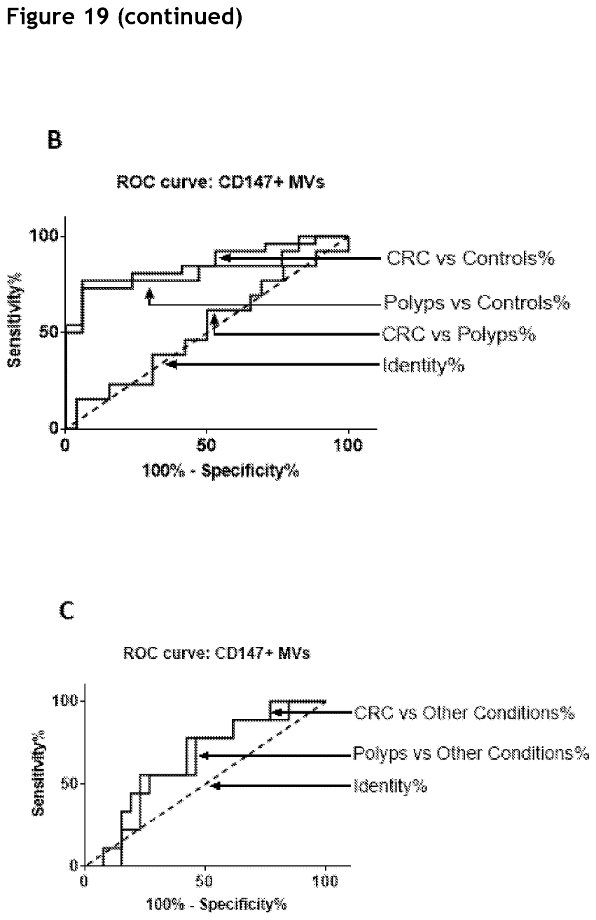

CD147+ MVs were significantly elevated in both the CRC (245855 (5568-794982) MVs/ml; p<0.001) and polyp (222248 (8189-1129090) MVs/ml; p<0.01) groups compared to healthy controls (59987 (2620-228095) MVs/ml; FIG. 19). Patients with other bowel conditions also had raised CD147+ (204068 (69770-553900) MVs/ml) compared to healthy controls, though this difference was not significant. ROC curve analyses showed that the sensitivity of this marker was moderate for detecting both colorectal cancer (60% sensitivity at a threshold of 186667 MVs/ml; AUC=0.83, p=0.0002) and polyps (62.5% sensitivity at a threshold of 186667 MVs/ml; AUC=0.8375, p=0.0006) from healthy control subjects.

### Example 14: Plasma Levels of Microvesicles Positive for PECAM1

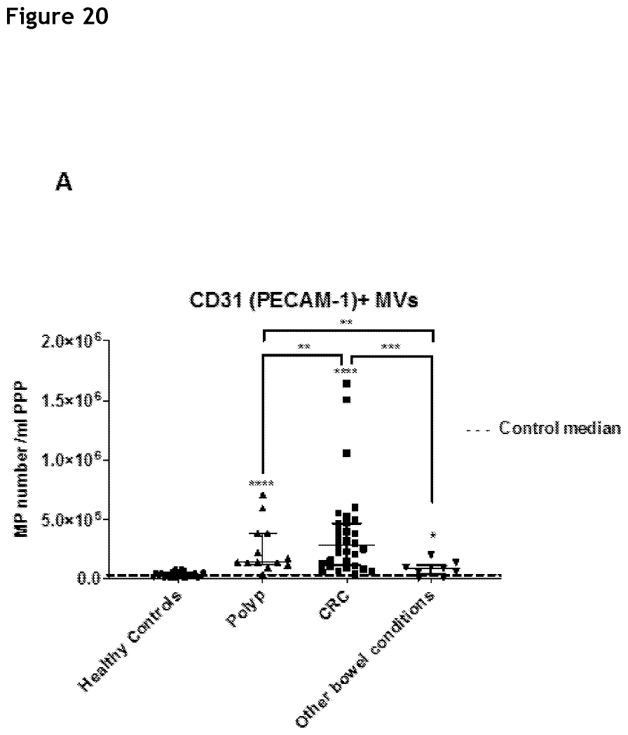

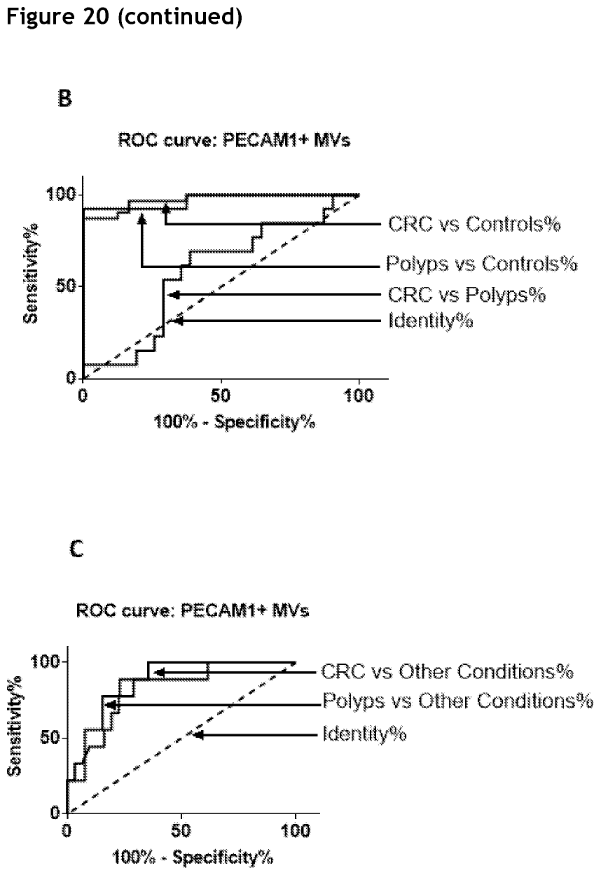

PECAM1+ MVs were significantly elevated in the CRC (263876 (41299-1643337) MVs/ml; n=30, p<0.0001) and polyp (139513 (39854-708464) MVs/ml; n=16, p<0.0001) groups compared to healthy controls (31514 (5900-82597) MVs/ml; n=24; FIG. 20). ROC curve analyses revealed high sensitivity and accuracy in detecting CRC patients (86.67% sensitivity at a threshold of 78953 MVs/ml; AUC=0.9722, p<0.0001) and patients with polyps (93.75% at a threshold of 78953MVs; AUC=0.9766, p<0.0001) from healthy controls. Sensitivity was poor for detecting CRC from polyp patients (6.25% sensitivity; AUC=0.5938; p=0.2993). PECAM1+MV levels were significantly higher in CRC patients compared to patients with other bowel conditions (61774 (4762-200246) MVs/ml; n=7; p<0.05), though sensitivity was only 57.14% used to detect CRC (AUC=0.8738, p=0.0023) from other bowel conditions. Such an elevation in MVs was not seen in the polyp group.

### Example 15: Plasma Levels of Microvesicles Positive for CD42a

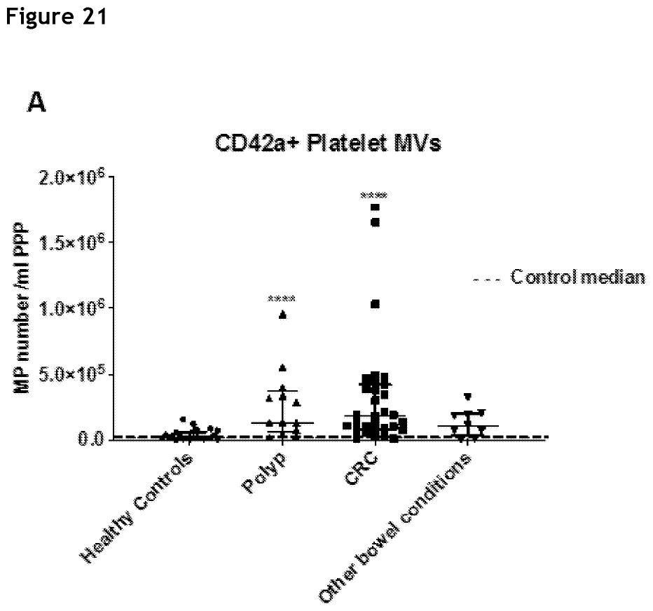

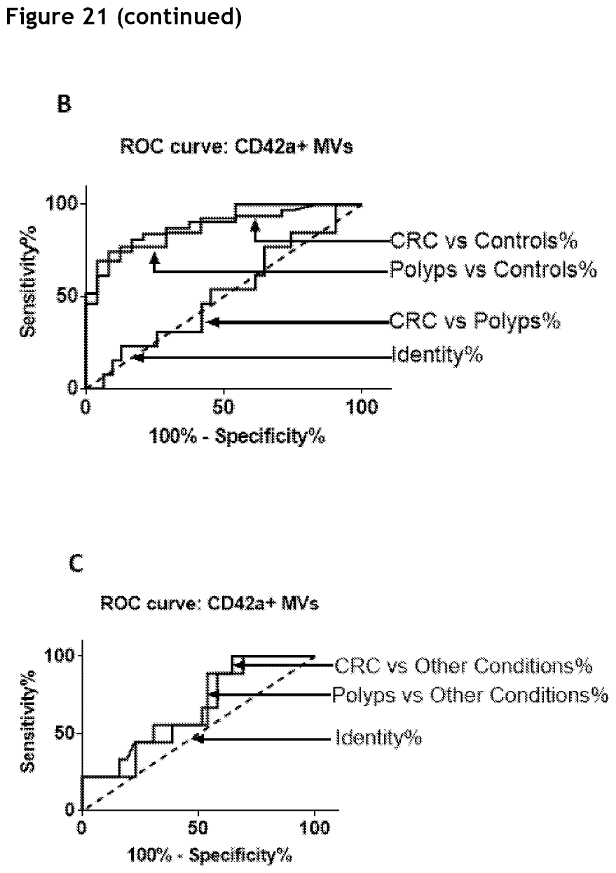

CD42a+ platelet MVs were significantly elevated in the CRC (186785 (9370-1771274) MVs/ml; n=30, p<0.0001) and polyp (162549 (18737-958529) MVs/ml; n=16, p<0.001) groups compared to healthy controls (21170 (2222-154783) MVs/ml; n=24; FIG. 21). Levels of these markers were lower in other bowel conditions (76350 (1905-323448) MVs/ml; n=7) compared to the CRC and polyp groups, though this was not significant. ROC curve analyses showed that these platelet MVs could significantly identify both colorectal cancer and precancerous patients from healthy controls with moderately high sensitivity (73.33% sensitivity at threshold 89912 MVs/ml and 75% sensitivity at 103680 MVs/ml, respectively) and high overall accuracy (AUC=0.8778, p<0.0001 and AUC=0.9036, p<0.0001, respectively). These markers were poor at distinguishing cancer from polyp (6.25% sensitivity, AUC=0.5104, p=0.9082) and identifying patients with CRC and polyps from patients with other bowel conditions (28.57% sensitivity, AUC=0.7262, p<0.0655 and 28.57% sensitivity, AUC=0.7321, p<0.0824, respectively).

### Example 16: Plasma Levels of PECAM11+/CD42a− Microvesicles

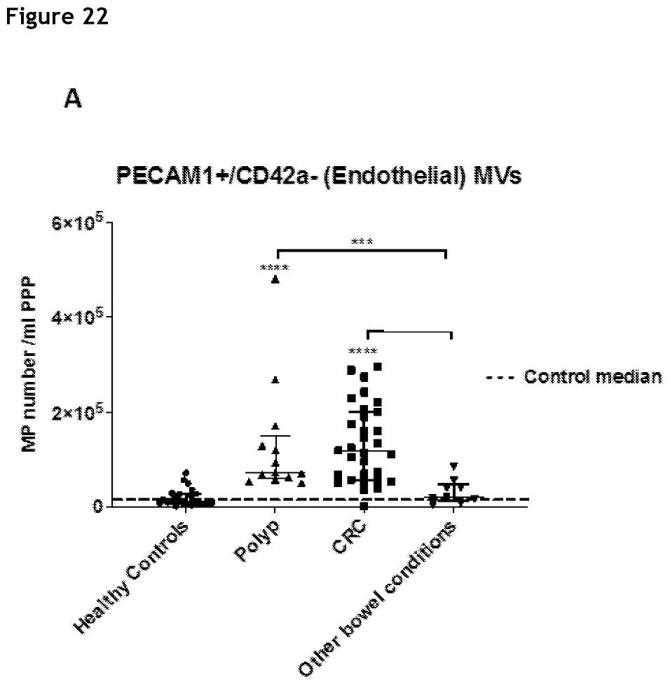

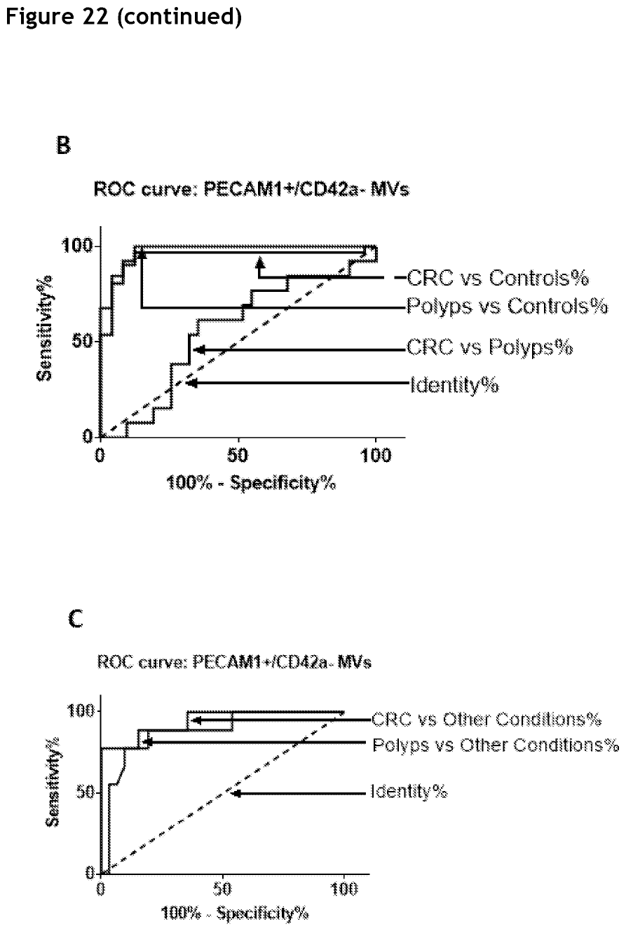

PECAM1+/CD42a− endothelial specific MVs were elevated in the CRC (117147 (2890-295951) MVs/ml; n=30, p<0.0001) and polyps (79823 (50535-481113) MVs/ml; n=16, p<0.0001) groups compared to the healthy control group (13399 (2429-72880) MVs/ml; n=24) and the other bowel conditions group (20129 (4127-41646) MVs/ml; n=7; FIG. 22). Sensitivity was high when using PECAM1+/CD42a− MVs to identify patients with CRC (90% at threshold of 51254 MVs/ml at >90% specificity; AUC=0.9458, p<0.0001) and polyps (93.75% at threshold of 52751 MVs/ml at >90% specificity; AUC=0.9714, p<0.0001) from healthy controls. Additionally, this marker showed high sensitivity and overall accuracy for detecting patients with CRC (100% sensitivity at threshold 40952 MVs/ml, AUC=0.95, p=0.0002) and polyps (100% sensitivity at threshold 52684 MVs/ml, AUC=1, p=0.0002) from other bowel conditions.

### Example 17: Levels of Serum Creatinine

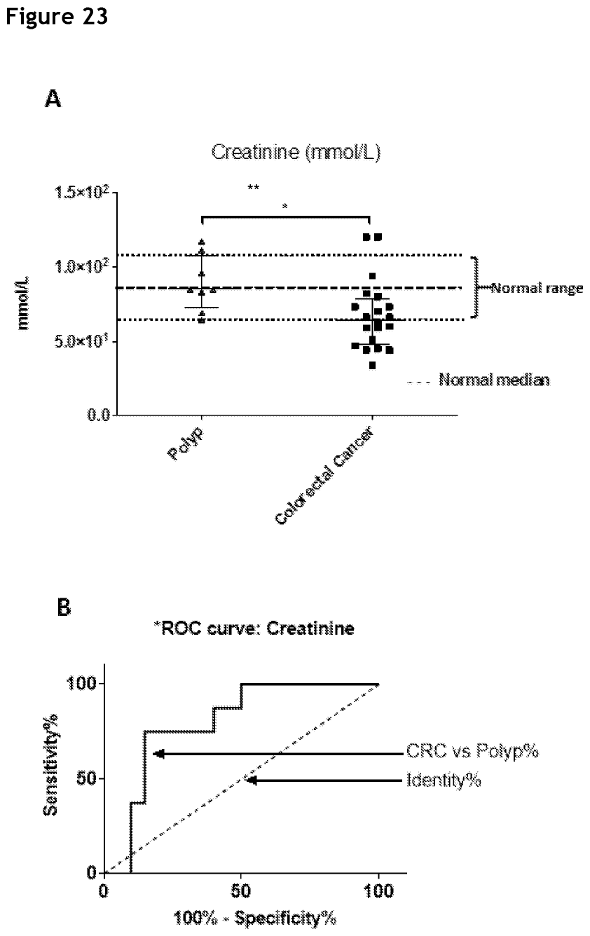

Serum creatinine was within normal range in patients with polyps (85.5 (64-120) mmol/L; n=12), while levels were significantly lower in CRC patients (64.5 (34-120) mmol/L; n=20, p<0.01; FIG. 23). Low creatinine levels were also seen in other bowel conditions (68.5 (58-73) mmol/L; n=3) though this was not significantly different from the other levels, possibly due to low sample size. ROC curve analysis showed that these markers could significantly identify CRC patients from the polyp group with moderate sensitivity at 75% at a threshold of 82.5 mmol/L with specificity at >90% (AUC=0.8354; p<0.0017).

### Example 18: Levels of Serum Albumin

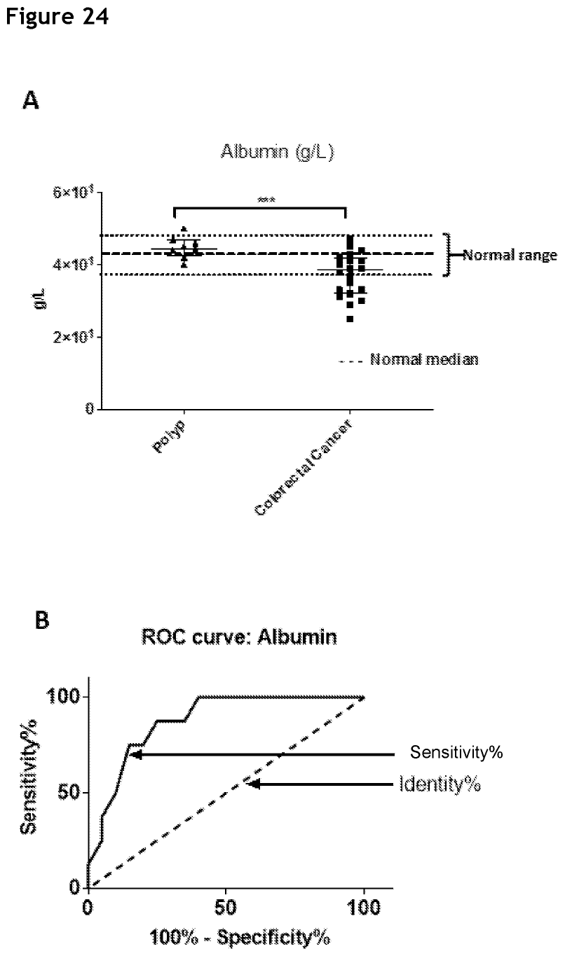

Serum albumin was significantly lower in CRC patients (38.5 (25-47) g/L; n=20, p<0.001) compared to patients with polyps (44 (40-50) g/L; n=12). Levels were within normal range and not lowered in patients with other bowel conditions (41.5 (31-45) g/L; n=4; FIG. 24). Albumin levels identified CRC from patients with polyps with moderately high sensitivity of 76.92% (AUC=0.8923; p=0.0002) at a threshold of 43.5 g/L at >90% specificity.

### Example 19: Levels of C-Reactive Protein

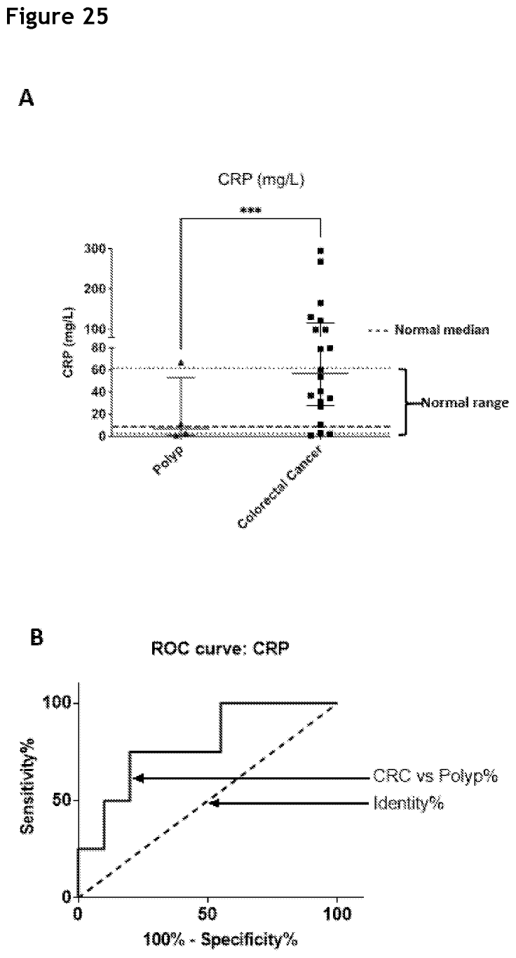

C-reactive protein levels were significantly elevated in CRC patients (69.35 (0.6-295.2) mg/L; n=16, p<0.001) compared to patients with polyps (0.5 (0.5-66.7) mg/L; n=8; FIG. 25). ROC curve analyses showed that CRP could significantly detect CRC patients from the polyp group with high sensitivity and overall accuracy (85.71% with >90% specificity at a threshold of 19.65 mg/L; AUC=0.881, p<0.0037).

### Example 20: Levels of Haemoglobin

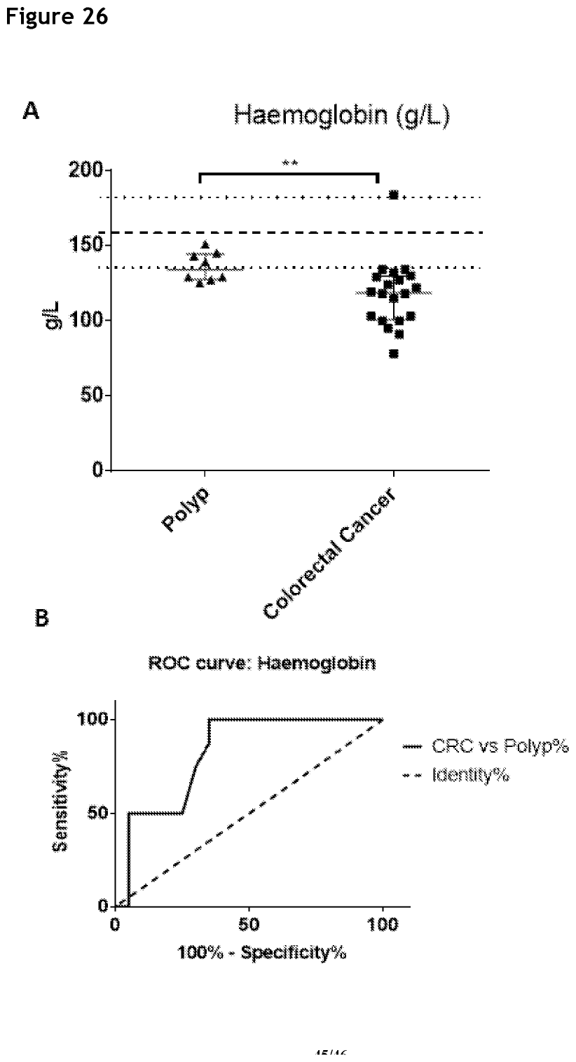

Haemoglobin levels were significantly lower in CRC patients (118 (78-184) g/L; n=20, p<0.001) compared to patients with polyps (129 (125-151) g/L; n=12; FIG. 26). Patients with other bowel conditions also had reduced levels (120 (101-168) g/L; n=4), though a significant difference was not seen compared to the other groups possibly due to low sample size. Haemoglobin levels were significantly able to distinguish patients with colorectal cancer from polyps with low sensitivity and high overall accuracy (58.33% with a threshold of 133 g/L at >90% specificity; AUC=0.8688, p=0.0006).

### Example 21: Levels of Serum Urea

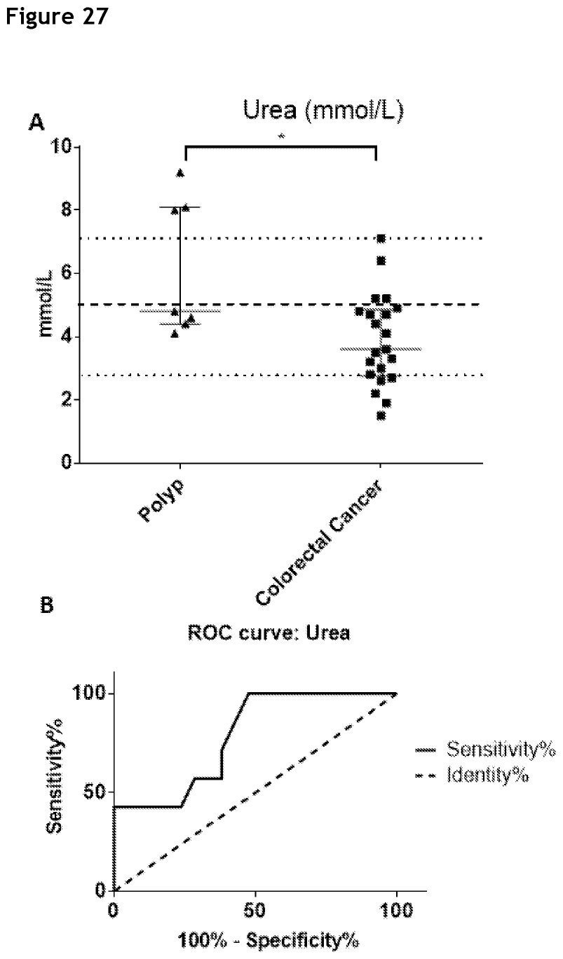

Patients with polyps had elevated serum urea levels (5.8 (4.1-9.2) mmol/L; n=11) relative to normal range, while reduced levels were seen in CRC patients (3.55 (1.5-7.1) mmol/L; n=20; FIG. 27). Serum urea was significantly lower in CRC compared to the polyp group (p<0.01). Patients with bowel conditions were within normal range (5.2 (3.8-5.2) mmol/L). ROC curve analyses showed these markers could significantly identify with 54.55% sensitivity at >90% specificity at a threshold of 5.5 mmol/L with high overall accuracy (AUC=0.8318; p=0.0026).

### Example 22: Plasma Level of Total Microvesicles in Benign Colorectal Polyps and Colorectal Cancer, and Plasma Levels of Microvesicles Positive for CEA, A33, LGR5, EPHB2, ICAM-1, CD31, CD42a, CD31+/CD42a−, CK20, CK7, CD20+/CD7−, HLA-DR+ and CD147

Tables 11 and 12 represent results using the same methodology as Examples 4 to 13 and 15, but with a higher sample size. Table 11 sets out the statistical parameters for detecting BRCP and Table 12 does the same for CRC.

TABLE 11
Predicting benign polyps

| Biomarker | AUC | 95% CI | Cut-off point | | Sensitivity | Specificity | p-value |
|---|---|---|---|---|---|---|---|
| Total MVs | 0.99 | 0.96-1.00 | >244 | MVs/μL | 100% | 93% | <0.0001 |
| CEA | 0.84 | 0.71-0.98 | >6.37 | MVs/μL | 100% | 40% | 0.001 |
| A33 | 0.68 | 0.48-0.88 | — | | — | — | 0.08 |
| LGR5 | 0.93 | 0.85-1.00 | >28.4 | MVs/μL | 100% | 60% | <0.0001 |
| EPHB2 | 0.89 | 0.78-1.00 | >1.07 | MVs/μL | 100% | 47% | <0.0001 |
| ICAM-1 | 0.79 | 0.63-0.96 | >3.6 | MVs/μL | 100% | 13% | 0.006 |
| CD31 | 0.99 | 0.97-1.00 | >72 | MVs/μL | 100% | 87% | <0.0001 |
| CD42a | 0.90 | 0.79-1.00 | >25 | MVs/μL | 100% | 47% | <0.0001 |
| CD31+/CD42a− | 0.98 | 0.95-1.00 | >44 | MVs/μL | 100% | 87% | <0.0001 |
| CK20 | 0.66 | 0.47-0.86 | — | | — | — | 0.12 |
| CK7 | 0.90 | 0.78-1.00 | >11 | MVs/μL | 100% | 33% | <0.0001 |
| CD20+/CD7− | 0.39 | 0.19-0.60 | — | | — | — | 0.30 |
| HLA-DR+ | 0.84 | 0.68-0.99 | >16 | MVs/μL | 100% | 7% | 0.001 |
| CD147 | 0.84 | 0.70-0.99 | >7.4 | MVs/μL | 100% | 13% | 0.001 |

AUC: area under the receiver operating curve. BCRP: benign colorectal polyps. CRC: colorectal cancer. CI: confidence interval. MVs: microvesicles.

TABLE 12
Predicting colorectal cancer

| Biomarker | AUC | 95% CI | Cut-off point | | Sensitivity | Specificity | P value |
|---|---|---|---|---|---|---|---|
| Total MVs | 0.92 | 0.83-1.00 | >145 | MVs/μL | 100% | 40% | <0.0001 |
| CEA | 0.96 | 0.90-1.00 | >10 | MVs/μL | 100% | 60% | <0.0001 |
| A33 | 0.87 | 0.75-0.99 | >13 | MVs/μL | 100% | 13% | <0.0001 |
| LGR5 | 0.87 | 0.76-0.98 | >19 | MVs/μL | 96% | 53% | <0.0001 |
| EPHB2 | 0.84 | 0.72-0.97 | >52 | MVs/μL | 100% | 13% | <0.0001 |
| ICAM-1 | 0.90 | 0.80-1.00 | >12 | MVs/μL | 96% | 47% | <0.0001 |
| CD31 | 0.97 | 0.93-1.00 | >38 | MVs/μL | 100% | 60% | <0.0001 |
| CD42a | 0.86 | 0.75-0.98 | >9 | MVs/μL | 100% | 20% | <0.0001 |
| CD31+/CD42a− | 0.95 | 0.87-1.00 | >38 | MVs/μL | 96% | 87% | <0.0001 |
| CK20 | 0.76 | 0.61-0.92 | >8 | MVs/μL | 100% | 47% | 0.006 |
| CK7 | 0.93 | 0.85-1.00 | >16 | MVs/μL | 100% | 47% | <0.0001 |
| CD20+/CD7− | 0.75 | 0.60-0.91 | >4 | MVs/μL | 100% | 33% | 0.009 |
| HLA-DR+ | 0.89 | 0.80-0.99 | >26 | MVs/μL | 100% | 27% | <0.0001 |
| CD147 | 0.85 | 0.73-0.97 | >17 | MVs/μL | 96% | 27% | <0.0001 |

AUC: area under the receiver operating curve. BCRP: benign colorectal polyps. CRC: colorectal cancer. CI: confidence interval. MVs: microvesicles.

1. A method of assessing whether an individual has colorectal cancer (CRC) or is at risk of developing CRC, the method comprising:
1) providing a sample of microvesicles (MVs) which have been obtained from the plasma of the individual;
2) determining the concentration of MVs in the individual's plasma; and
3) classifying the individual as having benign colorectal polyps (BCRPs) or CRC when the concentration of MVs in the individual's plasma is statistically significantly higher compared to control.

2. (canceled)

3. A method according to claim 1, wherein:
when the plasma concentration of MVs is 144 MVs/μL or more the individual is identified as having BCRPs or CRC, and wherein the method has a receiver operating characteristics (ROC) sensitivity of 100% and a specificity of 59%; or
when the plasma concentration of MVs is 244 MVs/μL or more the individual is identified as having BCRPs or CRC, and wherein the method has a ROC sensitivity of 100% and a specificity of 93%.

4. (canceled)

5. A method according to claim 1, further comprising:
1) determining the concentration of MVs in the individual's plasma which test positive for the detectable expression, preferably surface expression, of one or more biomarkers; and
2) classifying the individual as having BCRPs or CRC when the concentration of biomarker positive MVs in the individual's plasma is statistically significantly higher compared to control and/or when the concentration of biomarker positive MVs in the individual's plasma exceeds a threshold value.

6. A method according to claim 5, wherein:
1) the one or more biomarkers comprises CEA, and wherein the individual is classified as having CRC when the concentration of MVs which test positive for the detectable expression of CEA exceeds a threshold value, and wherein the method has a ROC sensitivity of 100% and a specificity of 60% or more, preferably wherein the threshold value is 10 MVs/μL or more; and/or
2) the one or more biomarkers comprises A33, and wherein the individual is classified as having CRC when the concentration of MVs which test positive for the detectable expression of A33 exceeds a threshold value, and wherein the method has a ROC sensitivity of 100% and a specificity of 13% or more, preferably wherein the threshold value is 13 MVs/μL or more; and/or
3) the one or more biomarkers comprises LGR5, and wherein the individual is classified as having CRC when the concentration of MVs which test positive for the detectable expression of LGR5 exceeds a threshold value, and wherein the method has a ROC sensitivity of 96% or more and a specificity of 53% or more, preferably wherein the threshold value is 19 MVs/μL or more; and/or
4) the one or more biomarkers comprises EPhB2, and wherein the individual is classified as having CRC when the concentration of MVs which test positive for the detectable expression of EPhB2 exceeds a threshold value, and wherein the method has a ROC sensitivity of 100% and a specificity of 13% or more, preferably wherein the threshold value is 52 MVs/μL or more; and/or
5) the one or more biomarkers comprises ICAM-1, and wherein the individual is classified as having CRC when the concentration of MVs which test positive for the detectable expression of ICAM-1 exceeds a threshold value, and wherein the method has a ROC sensitivity of 96% or more and a specificity of 47% or more, preferably wherein the threshold value is 12 MVs/μL or more; and/or
6) the one or more biomarkers comprises CD31, and wherein the individual is classified as having CRC when the concentration of MVs which test positive for the detectable expression of CD31 exceeds a threshold value, and wherein the method has a ROC sensitivity of 100% and a specificity of 60% or more, preferably wherein the threshold value is 38 MVs/μL or more; and/or
7) the one or more biomarkers comprises CD42a, and wherein the individual is classified as having CRC when the concentration of MVs which test positive for the detectable expression of CD42a exceeds a threshold value, and wherein the method has a ROC sensitivity of 100% and a specificity of 20% or more, preferably wherein the threshold value is 9 MVs/μL or more; and/or
8) the one or more biomarkers comprises CD31+/CD42a−, and wherein the individual is classified as having CRC when the concentration of MVs which test positive for the detectable expression of CD31+/CD42a− exceeds a threshold value, and wherein the method has a ROC sensitivity of 96% or more and a specificity of 87% or more, preferably wherein the threshold value is 38 MVs/μL or more; and/or
9) the one or more biomarkers comprises CK20, and wherein the individual is classified as having CRC when the concentration of MVs which test positive for the detectable expression of CK20 exceeds a threshold value, and wherein the method has a ROC sensitivity of 100% and a specificity of 47% or more, preferably wherein the threshold value is 8 MVs/μL or more; and/or
10) the one or more biomarkers comprises CK7, and wherein the individual is classified as having CRC when the concentration of MVs which test positive for the detectable expression of cytokeratin 7 exceeds a threshold value, and wherein the method has a ROC sensitivity of 100% and a specificity 47% or more, preferably wherein the threshold value is 16 MVs/μL or more; and/or
11) the one or more biomarkers comprises CK20+/CK7−, and wherein the individual is classified as having CRC when the concentration of MVs which test positive for the detectable expression of Ck20+/CD7− exceeds a threshold value, and wherein the method has a ROC sensitivity of 100% and a specificity of 33% or more, preferably wherein the threshold value is 4 MVs/μL or more; and/or
12) the one or more biomarkers comprises HLA-DR, and wherein the individual is classified as having CRC when the concentration of MVs which test positive for the detectable expression of HLA-DR exceeds a threshold value, and wherein the method has a ROC sensitivity of 100% and a specificity of 27% or more, preferably wherein the threshold value is 26 MVs/μL or more; and/or
13) the one or more biomarkers comprises CD147, and wherein the individual is classified as having CRC when the concentration of MVs which test positive for the detectable expression of CD147 exceeds a threshold value, and wherein the method has a ROC sensitivity of 96% or more and a specificity of 27% or more, preferably wherein the threshold value is 17 MVs/μL or more;
wherein:
1) the one or more biomarkers comprises CEA, and wherein the individual is classified as having BCRPs when the concentration of MVs which test positive for the detectable expression of CEA exceeds a threshold value, and wherein the method has a ROC sensitivity of 100% and a specificity of 40% or more, preferably wherein the threshold value is 6.37 MVs/μL or more; and/or
2) the one or more biomarkers comprises LGR5, and wherein the individual is classified as having BCRPs when the concentration of MVs which test positive for the detectable expression of LGR5 exceeds a threshold value, and wherein the method has a ROC sensitivity of 100% and a specificity of 60% or more, preferably wherein the threshold value is 28.4 MVs/μL or more; and/or
3) the one or more biomarkers comprises EPhB2, and wherein the individual is classified as having BCRPs when the concentration of MVs which test positive for the detectable expression of EPhB2 exceeds a threshold value, and wherein the method has a ROC sensitivity of 100% and a specificity of 47% or more, preferably wherein the threshold value is 1.07 MVs/μL or more; and/or
4) the one or more biomarkers comprises ICAM-1, and wherein the individual is classified as having BCRPs when the concentration of MVs which test positive for the detectable expression of ICAM-1 exceeds a threshold value, and wherein the method has a ROC sensitivity of 100% and a specificity of 13% or more, preferably wherein the threshold value is 3.6 MVs/μL or more; and/or
5) the one or more biomarkers comprises CD31, and wherein the individual is classified as having BCRPs when the concentration of MVs which test positive for the detectable expression of CD31 exceeds a threshold value, and wherein the method has a ROC sensitivity of 100% and a specificity of 87% or more, preferably wherein the threshold value is 72 MVs/μL or more; and/or
6) the one or more biomarkers comprises CD42a, and wherein the individual is classified as having BCRPs when the concentration of MVs which test positive for the detectable expression of CD42a exceeds a threshold value, and wherein the method has a ROC sensitivity of 100% and a specificity of 47% or more, preferably wherein the threshold value is 25 MVs/μL or more; and/or
7) the one or more biomarkers comprises CD31+/CD42a−, and wherein the individual is classified as having BCRPs when the concentration of MVs which test positive for the detectable expression of CD31+/CD42a− exceeds a threshold value, and wherein the method has a ROC sensitivity of 100% and a specificity of 87% or more, preferably wherein the threshold value is 44 MVs/μL or more; and/or
8) the one or more biomarkers comprises CK7, and wherein the individual is classified as having BCRPs when the concentration of MVs which test positive for the detectable expression of CK7 exceeds a threshold value, and wherein the method has a ROC sensitivity of 100% and a specificity of 33% or more, preferably wherein the threshold value is 11 MVs/μL or more; and/or
9) the one or more biomarkers comprises HLA-DR, and wherein the individual is classified as having BCRPs when the concentration of MVs which test positive for the detectable expression of HLA-DR exceeds a threshold value, and wherein the method has a ROC sensitivity of 100% and a specificity of 7% or more, preferably wherein the threshold value is 17 MVs/μL or more; and/or
10) the one or more biomarkers comprises CD147, and wherein the individual is classified as having BCRPs when the concentration of MVs which test positive for the detectable expression of CD147 exceeds a threshold value, and wherein the method has a ROC sensitivity of 100% and a specificity of 13% or more, preferably wherein the threshold value is 7.4 MVs/μL or more;
wherein the individual is classified as having BCRPs when the concentration of MVs which test positive for the detectable expression of all biomarkers comprising the Component Factor 2 (CF2) group of biomarkers exceeds a threshold value and wherein the method has a ROC sensitivity of 100% and a specificity of 63% or more, preferably wherein the threshold value is 222 MVs/μL or more; and/or
wherein the individual is classified as having BCRPs when the concentration of MVs which test positive for the detectable expression of all biomarkers comprising the Component Factor 1 (CF1) group of biomarkers exceeds a threshold value and wherein the method has a ROC sensitivity of 100% and a specificity of 100%, preferably wherein the threshold value is 761 MVs/μL or more; and/or
wherein the individual is classified as having CRC when the concentration of MVs which test positive for the detectable expression of all biomarkers comprising the Component Factor 2 (CF2) group of biomarkers exceeds a threshold value and wherein the method has a ROC sensitivity of 100% and a specificity of 50% or more, preferably wherein the threshold value is 157 MVs/μL or more; and/or
wherein the individual is classified as having CRC when the concentration of MVs which test positive for the detectable expression of all biomarkers comprising the Component Factor 1 (CF1) group of biomarkers exceeds a threshold value and wherein the method has a ROC sensitivity of 100% and a specificity of 56% or more, preferably wherein the threshold value is 439 MVs/μL or more.

7. (canceled)

8. (canceled)

9. (canceled)

10. (canceled)

11. (canceled)

12. A method according to claim 1, the method further comprising:
(a) providing a sample of blood which has been obtained from the individual, determining the concentration of a protein in the individual's blood and classifying the individual as having CRC when the concentration of the protein is below a threshold value, wherein the protein is haemoglobin and wherein the method has a ROC sensitivity of 68% or more and a specificity of 100%; and/or
(b) providing a sample of blood which has been obtained from the individual, determining the concentration of a blood cell type in the individual's blood and classifying the individual as having CRC when the concentration of the blood cell type is above a threshold value, wherein the blood cell type is neutrophils and wherein the method has a ROC sensitivity of 47% or more and a specificity of 100%; and/or
(c) providing a sample of blood which has been obtained from the individual, determining the concentration of a blood cell type in the individual's blood and classifying the individual as having CRC when the concentration of the blood cell type is below a threshold value, wherein the blood cell type is lymphocytes and wherein the method has a ROC sensitivity of 47% or more and a specificity of 100%; and/or
(d) providing a sample of blood which has been obtained from the individual, determining the concentration of a protein in the individual's blood and classifying the individual as having CRC when the concentration of the protein is below a threshold value, wherein the protein is albumen and wherein the method has a ROC sensitivity of 63% or more and a specificity of 100%; and/or
(e) providing a sample of blood which has been obtained from the individual, determining the concentration of a protein in the individual's blood and classifying the individual as having CRC when the concentration of the protein is above a threshold value, wherein the protein is C-reactive protein (CRP) and wherein the method has a ROC sensitivity of 63% or more and a specificity of 100%; and/or
(f) providing a sample of blood which has been obtained from the individual, determining the concentration of a compound in the individual's blood and classifying the individual as having CRC when the concentration of the compound is below a threshold value, wherein the compound is urea and wherein the method has a ROC sensitivity of 58% or more and a specificity of 100%; and/or
(g) providing a sample of blood which has been obtained from the individual, determining the concentration of a compound in the individual's blood and classifying the individual as having CRC when the concentration of the compound is below a threshold value, wherein the compound is creatinine and wherein the method has a ROC sensitivity of 53% or more and a specificity of 100%; and/or
(h) providing a sample of blood which has been obtained from the individual, determining the concentration of MVs in the plasma of the individual which test positive for the detectable expression of a protein and classifying the individual as having CRC when the concentration of MVs positive for the protein in the plasma is above a threshold value, wherein the protein is carcinoembryonic antigen (CEA) and wherein the method has a ROC sensitivity of 25% or more and a specificity of 100%.

13. A method according to claim 12(a), wherein the individual is distinguished as having CRC rather than BCRPs when the concentration of haemoglobin is 133 g/L or less, and wherein the method has a ROC sensitivity of 58.3% or more and a specificity of 90% or more; or wherein the individual is classified as having CRC when the concentration of haemoglobin is 124.5 g/L or less, and wherein the method has a ROC sensitivity of 68% or more and a specificity of 100%.

14. (canceled)

15. A method according to claim 12(b), wherein the individual is classified as having CRC when the concentration of neutrophils is 7.7×10⁹/L or more, and wherein the method has a ROC sensitivity of 47% or more and a specificity of 100%.

16. (canceled)

17. A method according to claim 12(c), wherein the individual is classified as having CRC when the concentration of lymphocytes is 1.16×10⁹/L or less, and wherein the method has a ROC sensitivity of 47% or more and a specificity of 100%.

18. (canceled)

19. A method according to claim 12(d), wherein the individual is distinguished as having CRC rather than BCRPs when the concentration of albumen is 43.5 g/L or less, and wherein the method has a ROC sensitivity of 76.9% or more and a specificity of 90% or more; or wherein the individual is classified as having CRC when the concentration of albumen is 39.5 g/L or less, and wherein the method has a ROC sensitivity of 63% or more and a specificity of 100%.

20. (canceled)

21. A method according to claim 12(e), wherein the individual is distinguished as having CRC rather than BCRPs when the concentration of CRP is 19.65 mg/L or more, and wherein the method has a ROC sensitivity of 85.7% or more and a specificity of 90% or more; or wherein the individual is classified as having CRC when the concentration of CRP is 72.75 mg/L or more, and wherein the method has a ROC sensitivity of 63% or more and a specificity of 100%.

22. (canceled)

23. A method according to claim 12(f), wherein the individual is distinguished as having CRC rather than BCRPs when the concentration of urea is 5.5 mmol/L or less, and wherein the method has a ROC sensitivity of 54.5% or more and a specificity of 90% or more; or wherein the individual is classified as having CRC when the concentration of urea is 3.85 mmol/L or less, and wherein the method has a ROC sensitivity of 58% or more and a specificity of 100%.

24. (canceled)

25. A method according to claim 12(g), wherein the individual is distinguished as having CRC rather than BCRPs when the concentration of creatinine is 82.5 mmol/L or less, and wherein the method has a ROC sensitivity of 75% or more and a specificity of 90.0% or more; or wherein the individual is classified as having CRC when the concentration of creatinine is 63.5 μmol/L or less, and wherein the method has a ROC sensitivity of 53% or more and a specificity of 100%.

26. (canceled)

27. A method according to claim 12(h), wherein the individual is classified as having CRC when the concentration of CEA-positive MVs in the plasma is 221 MVs/μL or more, and wherein the method has a ROC sensitivity of 25% or more and a specificity of 100%.

28. A method according to claim 5, wherein the expression of the one or more biomarkers is detected on the surface of MVs via an immunoassay, preferably an enzyme-linked immunosorbent assay (ELISA); or via a cytometry assay, such as a mass cytometry assay or a flow cytometry assay; or by a mass spectrometry assay; preferably the one or more biomarkers are detected via a flow cytometry assay, preferably using an anti-biomarker antibody conjugated to a fluorophore.

29. A method according to claim 12(a), wherein the protein and/or the blood cell type is detected via an immunoassay, preferably an enzyme-linked immunosorbent assay (ELISA); or via a cytometry assay, such as a mass cytometry assay or a flow cytometry assay; or by a mass spectrometry assay.

30. A method according to 12(f), wherein the compound is detected via a mass spectrometry assay or by nuclear magnetic resonance.

31. A method according to claim 1, wherein MVs are detected via an immunoassay, preferably an enzyme-linked immunosorbent assay (ELISA); or via a cytometry assay, such as a mass cytometry assay or a flow cytometry assay; or by a mass spectrometry assay; preferably MVs are detected via a flow cytometry assay, preferably using an anti-annexin V antibody conjugated to a fluorophore such as fluorescein isothiocyanate (FITC).

32. (canceled)

33. A kit comprising reagents for:
a) detecting microvesicles (MVs) and determining the concentration of MVs in an individual's plasma; and
b) detecting the presence of one, more or all biomarkers in a group of biomarkers expressed in MVs, wherein the group of biomarkers consists of CEA, A33, LGR5, EPhB2, ICAM-1, CD31, CD42a, CD31+/CD42a−, CK20, CK7, CK20+/CK7−, HLA-DR or CD147.

34. A kit according to claim 33, comprising:
(1) reagents for detecting the presence of the Component Factor 1 (CF1) group of biomarkers;
(2) reagents for detecting the presence of one, more or all of the biomarkers CEA, A33, CK20, CK7, CK20+/CK7−, HLA-DR and CD147;
(3) reagents for detecting the presence of the Component Factor 1 (CF2) group of biomarkers;
(4) reagents for detecting the presence of one, more or all of the biomarkers ICAM-1, CD31, CD42a, CD31+/CD42a−, CK20, CK7, CK20+/CK7−, HLA-DR and CD147; and/or
(5) reagents for detecting and quantifying in blood:
a) a blood protein, wherein the protein is haemoglobin, albumen, C-reactive protein (CRP) and/or carcinoembryonic antigen (CEA); and/or
b) a blood cell, wherein the cell is a neutrophil and/or a lymphocyte; and/or
c) a blood compound, wherein the compound is urea and/or creatinine.

35. (canceled)

36. (canceled)

37. (canceled)

38. (canceled)

39. A kit according to claim 33,
(1) wherein the reagent for detecting MVs and determining the concentration of MVs in an individual's plasma comprises an anti-Annexin V antibody, optionally conjugated to a fluorophore, preferably fluorescein isothiocyanate (FITC); and/or,
(2) wherein the reagents for detecting the presence of the one, more or all biomarkers expressed in MVs comprise respectively an anti-CEA antibody, an anti-A33 antibody, an anti-LGR5 antibody, an anti-EPhB2 antibody, an anti-ICAM-1 antibody, an anti-CD31 antibody, an anti-CD42a antibody, CD31+/CD42a−, an anti-CK20 antibody, an anti-CK7 antibody, CK20+/CK7−, an anti-HLA-DR antibody and an anti-CD147 antibody, preferably wherein the antibodies are labelled with a fluorophore; and/or
(3) wherein the reagents for detecting the presence of the one, more or all biomarkers expressed in MVs comprise:
a) forward and reverse primers for amplifying, in a nucleic acid amplification reaction, nucleic acid encoding the one or more biomarkers;
b) a detectable probe for detecting amplification products generated by the forward and reverse primers in the nucleic acid amplification reaction; and optionally
c) a nucleic acid amplification enzyme, preferably a DNA polymerase.

40. (canceled)

41. (canceled)

42. A kit according to claim 39(3), comprising a nucleic acid amplification enzyme, preferably a DNA polymerase, and further comprising a reverse transcriptase enzyme.

43. A chip comprising:
a) a first input channel for introduction of MVs;
b) a second input channel for introduction of reagents for determining the concentration of MVs in an individual's plasma, or determining the concentration of MVs in the individual's plasma which test positive for the detectable surface expression of one or more biomarkers;
c) a third input channel for introduction of wash buffer;
d) a first chamber for mixing the MVs with the reagents;
e) a second chamber for washing the MVs from the reagents using the wash buffer;
f) a first output channel for releasing MVs after mixing and washing, for introduction into a flow cytometer; and
g) a second output channel for releasing the reagents and wash buffer after mixing and washing.
